(19) United States
(12) Patent Application Publication
(10) Pub. No.: US 2026/0144924 A1
(43) Pub. Date: May 28, 2026

(54) Fluid Purification Compositions and Methods of Fluid Purification Using the Same

(71) Applicant: Temple University-Of The Commonwealth of Higher Education, Philadelphia, PA (US)

(72) Inventors: Rouzbeh Tehrani, Philadelphia, PA (US); Farbod Alimohammadi, Philadelphia, PA (US)

(21) Appl. No.: 19/107,389

(22) PCT Filed: Aug. 31, 2023
(86) PCT No.: PCT/US2023/031697

§ 371 Date: Feb. 27, 2025

Related U.S. Application Data
(60) Provisional application No. 63/374,093, filed on Aug. 31, 2022.

(51) Int. Cl. A61M 1/16; B01J 20/22; B01J 20/28; C08J 9/04

(52) U.S. Cl. CPC A61M 1/1654; A61M 1/1696; B01J 20/22; B01J 20/28059; B01J 20/28083; B01J 20/28085; C08J 9/04

(57) ABSTRACT

Described herein is a composition comprising porous particles, wherein the porous particles comprise an organic polymer and further comprise functional groups that selectively and reversibly bind blood components.

Also described herein is method of dispersing a medical composition in a filtration unit, the method comprising the steps of: providing a vessel comprising a medical composition; securing the vessel to a filtration unit; and depositing the composition into the filtration unit; wherein the medical composition comprises porous particles comprising at least one organic polymer; the filtration unit comprises a filtration membrane having hollow fiber pores; and the average diameter of the porous particles is larger than the hollow fiber pores of the filtration membrane.

Also described herein is a method of synthesizing polymer beads, the method comprising the steps of: dispersing a polymer in a solvent to give a polymer solution; adding a blowing agent to the polymer solution; and treating the polymer solution with a base, acid, or microwaves, to form polymer beads.

### CROSS-REFERENCE TO RELATED APPLICATIONS

The present application claims priority to U.S. Provisional Application No. 63/374,093, filed Aug. 31, 2022, which is incorporated by reference herein in its entirety.

### BACKGROUND OF THE INVENTION

In the United States, hemodialysis is the most common treatment for kidney failure. Although there have been significant improvements in the hemodialysis membrane biocompatibility and purity of dialysate, in-center hemodialysis still has worse outcomes compared to kidney transplantation and worse quality of life compared to peritoneal dialysis. Furthermore, each 4-hour treatment requires 156 liter of ultrapure dialysate and with over 500,000 people in the United States receiving thrice weekly treatments, 10 billion liters of water is required each year. This provides a significant toll on the environment in energy and water consumption, that can challenge care delivery for low- and middle-income countries. Additionally, this water consumption can be a significant barrier to home hemodialysis and the artificial wearable kidney. Hemodialysis with single pass large volume dialysate is the widely accepted technology; followed by low efficiency frequent dialysis, and hemodiafiltration. Sorbent hemodialysis, which recycles the dialysate, was a viable technology which used 6 L of water on average. Engineered materials at nano- and micrometer scales specific for fluid purification and drug delivery have shown promise in the field of dialysis.

There remains a need in the art for purification materials for the efficient purification of fluids. The present invention satisfies this unmet need.

### SUMMARY OF THE INVENTION

In one aspect, the present invention relates to a composition comprising porous particles, wherein the porous particles comprise an organic polymer and further comprise functional groups that selectively and reversibly bind blood components. In one embodiment, the porous particles have a core and a shell; and wherein the core and the shell have different porosity. In one embodiment, the porous particles have a Brunauer-Emmet-Teller surface area between 1.0 and 30 m²/g. In one embodiment, the organic polymer is selected from the group consisting of chitosan, cellulose, lignin, polyacrylic, polysulfone or it can be synthesized with monomers comprising a styrene, an acrylate, a divinylbenzene, and combinations, co-polymers, or block co-polymers thereof. In one embodiment, the porous particles have an average pore diameter between 5 and 35 nm. In one embodiment, the porous particles have an average diameter between 2 nm to 1000 μm. In one embodiment, the porous particles comprise at least one crosslinker. In one embodiment, the porous particles comprise a compound having a known affinity for a specific blood component. In one embodiment, the core of the porous particle comprises functional groups that selectively and reversibly bind specific electrolytes, proteins, metabolites, or enzymes; and the shell of the porous particles comprise at least one biocompatible polymer. In one embodiment, the composition further comprises saline and optionally comprises a dispersant.

In one aspect, the present invention relates to a kit for adding a purification composition to a dialysis unit, the kit comprising: the composition dispersed or suspended in saline; a syringe having a volume sufficient to contain the composition; and instructions for using the kit.

In one aspect, the present invention relates to a packed column comprising the composition comprising porous particles.

In one aspect, the present invention relates to a dialyzer comprising the composition comprising porous particles.

In one aspect, the present invention relates to a dialyzer, wherein the porous particles comprise functional groups that selectively and reversibly bind potassium (K) or phosphate (PO₄³⁻).

In one aspect, the present invention relates to a method of removing potassium or phosphate from the blood of a subject, the method comprising the steps of: providing the dialyzer and dialyzing the blood of the subject using the dialyzer.

In one aspect, the present invention relates to a method of dispersing a medical composition in a filtration unit, the method comprising the steps of: providing a vessel comprising a medical composition; securing the vessel to a filtration unit, and depositing the composition into the filtration unit; wherein the medical composition comprises porous particles comprising at least one organic polymer; the filtration unit comprises a filtration membrane having hollow fiber pores; and the average diameter of the porous particles is larger than the hollow fiber pores of the filtration membrane. In one embodiment, the vessel is a syringe. In one embodiment, the filtration unit is a dialyzer for the dialysis of blood and blood products.

In one aspect, the present invention relates to a system for the removal of a blood component from the blood of a patient, the system comprising: a dialyzer unit having a blood inlet, a blood outlet, and dialysate inlet, and a dialysate outlet; wherein the blood inlet and blood outlet are fluidly connected, and the dialysate inlet and the dialysate outlet are fluidly connected; a dialysate containment vessel which is fluidly connected to the dialysate inlet port via a filtration line and a bypass line; wherein the bypass line includes a bypass valve between the dialyzer unit and the dialysate vessel; wherein the filtration line includes a filtration cartridge fluidly connected to the dialysate containment vessel and the dialysate inlet and a valve between the purification cartridge and the dialysate containment vessel; and wherein the filtration cartridge comprises the composition comprising porous particles.

In one aspect, the present invention relates to a method of dialyzing the blood of a subject in need thereof, the method comprising the steps of: providing the system described; providing a dialysate solution; adding a blood component to the dialysate solution such that the blood component concentration of the dialysate solution is lower than the blood component level of the blood; passing the dialysate solution through the bypass line of the system and through the dialyzer until the blood component level of the blood and dialysate have equilibrated; and directing at least a portion of the dialysate solution through the purification cartridge. In one aspect, the present invention relates to a method of synthesizing polymer beads, the method comprising the steps of: dispersing a polymer in a solvent to give a polymer solution; adding a blowing agent to the polymer solution; and treating the polymer solution with a base, acid, or microwaves, to form polymer beads.

### BRIEF DESCRIPTION OF THE DRAWINGS

The following detailed description of various embodiments of the invention will be better understood when read in conjunction with the appended drawings. For the purpose of illustrating the invention, there are shown in the drawings, illustrative embodiments. It should be understood, however, that the invention is not limited to the precise arrangements and instrumentalities of the embodiments shown in the drawings.

FIG. 1 depicts a design concept for an engineered porous-shell and core system containing active binders (adsorbents), catalysts, and slow releasing desorbents.

FIG. 2 is an SEM image of a chitosan-based particle with a porous shell (cross-sectional view).

FIG. 3 depicts examples of particles and particles with fins.

FIG. 4 is an SEM image of a chitosan-based porous resin (cross-sectional view).

FIG. 5 is an SEM image of a porous-scaffold system, containing active binders (adsorbents), catalysts, and/or slow releasing desorbents.

FIG. 6 depicts an exemplary process for the injection of particles into a commercially available dialyzer.

FIG. 7 is a series of photographs showing exemplary particles in an OptiFlux dialyzer.

FIG. 8 is SEM image of a hollow fiber membrane fiber with schematics of particles.

FIG. 9 depicts an exemplary nano-slurry method of dialysis.

FIG. 10 depicts an exemplary beads+membrane method of dialysis in which the dialysate is recirculated.

FIG. 11 depicts an exemplary beads+membrane method of dialysis in which the dialysate is not recirculated.

FIG. 12 depicts an exemplary packed column dialysis in which the blood is placed in direct contact with the purification composition.

FIG. 13 depicts an exemplary packed column dialysis in which the dialysate is placed in direct contact with the purification composition.

FIG. 14 depicts a system for stepwise decrease of dialysate potassium concentration gradient while decreasing blood potassium.

FIG. 15 is an SEM image of a chitosan-based porous resin with macro pores and active sites on outer surface. Beads have micro, nano, and macro pores depending on design and application.

FIG. 16 is an SEM image of a chitosan-based porous resin with porous fins.

FIG. 17 is an image of a chitosan-based porous resin with porous fins, and an SEM image of a porous bead with fins.

FIG. 18 depicts a design concept for an engineered swellable gel-like drug delivery system containing active binders (adsorbents), catalysts, and slow releasing desorbents.

FIG. 19 depicts a design concept for an engineered swellable gel-like drug delivery system containing active binders (adsorbents), catalysts, and slow releasing desorbents with a polymer tail.

FIG. 20 depicts a column packed with various beads for dialysate purification. The column can also be used for hemoperfusion.

FIG. 21 depicts use of biocompatible chitosan-based resins for blood purification (hemoperfusion).

FIG. 22 depicts the experimental setup for an experiment using spiked dialysate.

FIG. 23 is a plot of potassium levels in a mock dialysis experiment. Blood-1-1: dialysate solution initially spiked with potassium (K⁺) to 8.2 mmol/L. This solution was dialyzed using a dialysate solution (3 mmol/L K⁺) and dialyzer with no materials added. Dialysate-1: dialysate solution used as a cleansing solution to dialyze Blood-1. Blood-2: a dialysate solution initially spiked with potassium (K⁺) to 8.2 mmol/L. This solution was dialyzed using a dialyzer filled with Nano-Dyle™ and dialysate solution (K⁺3.9 mmol/L). Dialysate-2: a dialysate solution having an initial K⁺ concentration of 3.9 mmol/L used cleansing solution to dialyze Blood-2.

FIG. 24 depicts the experimental setup for an experiment using porcine blood at an initial potassium concentration of 8.2 mmol/L.

FIG. 25 is a plot of potassium levels in a mock dialysis experiment using porcine blood and a dialyzer filled with Nano-Dyle™.

FIG. 26 is a second image of the plot showing the first 40 minutes of the experiment.

FIG. 27 depicts a packed column showing potential route of multiple passes, as well as a photograph of the beads in saline solution post-hemoperfusion.

FIG. 28 is a schematic of the droplet method for bead synthesis.

FIG. 29 is a scheme showing an exemplary preparation of porous polymer beads.

FIG. 30 depicts an exemplary composition for the delivery of non-biocompatible compounds.

FIG. 31 is a series of SEM images of beads prepared with azodicarboxamide.

FIG. 32 is a series of SEM images of beads prepared with azodicarboxamide and an organic phase.

FIG. 33 is a series of SEM images of beads prepared with an organic phase.

FIG. 34 is a series of SEM images of beads prepared with organic phase and lanthanum chloride.

FIG. 35 provides an elemental analysis of beads prepared with organic phase and lanthanum chloride.

FIG. 36 is a series of SEM images of low molecular weight chitosan with Lanthanum, carbonized (pyrolyzed) at 800° C. in nitrogen gas.

FIG. 37 is a series of SEM images of a chitosan scaffold for activated carbon, charcoal, or clay-based adsorbents.

FIG. 38 is a series of SEM images of beads prepared with organic phase and sodium zirconium cyclosilicate.

FIG. 39 provides an elemental analysis of beads prepared with organic phase and sodium zirconium cyclosilicate.

FIG. 40 is a is an overlay of XRD diffractograms of porous chitosan/Lokelma, Lokelma, and chitosan.

FIG. 41 is a SEM image of porous chitosan/Lokelma, followed by an EDX of porous chitosan/Lokelma.

FIG. 42 is a SEM image of porous chitosan/clinoptilolite, followed by an EDX of porous chitosan/clinoptilolite.

FIG. 43 is a SEM image of obtained chitosan solution which was sprayed into a NaOH aqueous solution.

FIG. 44 is a series of SEM images of beads prepared with organic phase and followed by carbonization.

FIG. 45 is an SEM image showing the surface and pore structure of a bead prepared with organic phase and followed by carbonization.

FIG. 46 is a series of SEM images of beads prepared with organic phase and lanthanum chloride and followed by carbonization.

FIG. 47 provides an elemental analysis of beads prepared with organic phase and lanthanum chloride and followed by carbonization.

FIG. 48, comprising FIGS. 48A-48F are a series of SEM images. FIGS. 48A and 48B are SEM images of porous chitosan. FIGS. 48C and 48D are SEM images of porous chitosan/Lokelma. FIGS. 48E and 48F are SEM images of porous chitosan/Clinoptilolite.

FIG. 49, comprising FIGS. 49A-49C, depicts Brunauer-Emmet-Teller (BET) results.

FIG. 49A is a plot of BET results for porous chitosan, porous chitosan/Lokelma, and porous chitosan/Clinoptilolite. FIG. 49B is a plot of non-porous chitosan. FIG. 49C is a table compiling BET results.

FIG. 50 is a plot of thermogravimetric analysis (TGA) of particles.

FIG. 51 is a plot of potassium uptake of particle in the dialysate solution.

FIG. 52 is a plot of potassium removal in porcine blood by chitosan particles.

FIG. 53 is a plot of calcium removal in porcine blood by porous chitosan/Lokelma and porous chitosan/Clinoptilolite.

FIG. 54, comprising FIGS. 54A and 54B, depicts hemoperfusion. FIG. 54A depicts a syringe used for hemoperfusion experiments. FIG. 54B depicts a table of hemoperfusion experiment results.

FIG. 55, comprising FIGS. 55A-55D, are SEM and fluorescent images of microspheres. FIG. 55A depicts SEM images of non-porous microspheres. FIG. 55B depicts SEM and FM images of PCP-M microspheres. FIG. 55C depicts SEM and FM images of PCP-T microspheres. FIG. 55D depicts SEM and FM images of PCP-A microspheres.

FIG. 56 is plot of nitrogen adsorption and desorption curve of the chitosan particles.

FIG. 57 is an XRD diffractogram of non-porous chitosan microspheres, fine tunes chitosan microspheres, namely PCP-M, PCP-T, and PCP-A, and powdered chitosan.

FIG. 58 depicts FTIR spectra of the porous chitosan microspheres and powdered chitosan.

FIG. 59 comprising FIGS. 59A and 59B, are TGA curves of chitosan particles. 59A depicts a thermogravimetric analysis of the chitosan particles. 59B depicts a derivative thermogravimetry of chitosan particles.

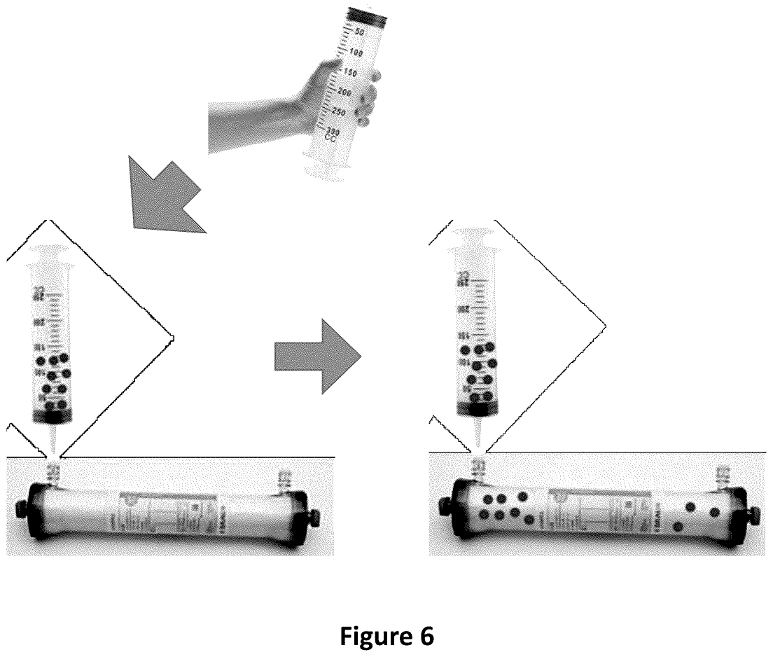

FIG. 6O, comprising FIGS. 60A-60B, are plots for methylene blue removal, and adsorption kinetic for non-porous chitosan particles. FIG. 60A depicts methylene blue removal from the solution by non-porous chitosan particles. FIG. 60B depicts the adsorption kinetic of pseudo-first and second-order reactions for non-porous chitosan.

FIG. 61, comprising FIGS. 61A-61C, are plots of methylene blue removal from the solution by chitosan particles. FIG. 61A depicts methylene blue removal from the solution by chitosan PCP-M particles. FIG. 61B depicts methylene blue removal from the solution by chitosan PCP-T particles. FIG. 61C depicts methylene blue removal from the solution by chitosan PCP-A particles.

FIG. 62, comprising FIGS. 62A-62C, are plots of Freundlich and Langmuir isotherm calculation of PCP-M, PCP-T, and PCP-A. FIG. 62A depicts Freundlich and Langmuir isotherm calculation of PCP-M. FIG. 62B depicts Freundlich and Langmuir isotherm calculation of PCP-T. FIG. 62C depicts Freundlich and Langmuir isotherm calculation of PCP-A.

FIG. 63, comprising FIGS. 63A-63C, are plots of the adsorption kinetic of pseudo-first and second-order reaction for PCP-M, PCP-T, and PCP-A. FIG. 63A depicts the adsorption kinetic of pseudo-first and second-order reactions for PCP-M. FIG. 63B depicts the absorption kinetic of pseudo-first and second-order reaction for PCP-T. FIG. 63C depicts the adsorption kinetic of pseudo-first and second-order reaction for PCP-A.

FIG. 64, comprising FIGS. 64A-64E, depicts the setup of the microspheres in the MicroTesting unit. FIG. 64A is an in-situ microtesting experimental setup. FIG. 64B is a non-porous microsphere after material failure. FIG. 64C is PCP-M after material failure. FIG. 64D is PCP-A after material failure. FIG. 64E is PCP-T after material failure.

FIG. 65, comprising FIGS. 65A-65D, presents nominal stress vs. nominal strain plots used to determine the deformation resistance (DR) of the synthesized materials. FIG. 65A is a plot of the non-porous microsphere. FIG. 65B is a plot of the PCP-M, PCP-A, and PCP-T microspheres. FIG. 65C is a plot of the soaked non-porous microsphere. FIG. 65D is a plot of the soaked PCP-M, PCP-A, and PCP-T microspheres.

FIG. 66 is a series of images of polymeric gels and excess sevelamer carbonate used in a dialyzer.

FIG. 67 is a plot depicting precision treatment/removal management of phosphate using sevelamer carbonate with dialysate.

FIG. 68 is a series of photos depicting a dialysate slurry and Fresenius Optiflux dialyzer.

FIG. 69 is a benchtop mock hemodialysis experimental setup.

FIG. 70, comprising FIGS. 70A-70B depict a series of testing for leaching of nanomaterials. FIG. 70A depicts oven-dried samples after centrifugation. FIG. 70B depicts oven-dried blood samples without centrifugation. FIG. 70C depicts an example of EDS analysis of ground dried blood samples. No elements of adsorbent composition were detected in blood.

FIG. 71, comprising FIGS. 71A and 71B, depicts plots of creatine removal. FIG. 71A depicts creatine removal of conventional single-pass dialysis (control). FIG. 71B depicts creatine removal of single-pass nanoslurry dialysate.

FIG. 72, comprising FIGS. 72A and 72B, depicts plots with impact of high flow rates and dilution on analytes at equilibrium due to recirculation of dialysate. FIG. 72A depicts creatine dilution. 72B depicts BUN (Blood urea nitrogen) dilution.

FIG. 73, comprising FIGS. 73A-73B, is a series of plots depicting creatinine removal at high flow rates (300/400 ml·min⁻¹). FIG. 73A depicts trial 1 of creatinine high flow rate study. FIG. 73B depicts trial 2 of creatinine high flow rate study using nano-slurry recirculated dialysate.

FIG. 74, comprising FIGS. 74A-74D, is a series of plots depicting phosphate and BUN management. FIG. 74A depicts trial 1 of phosphate management study. FIG. 74B depicts trial 2 of phosphate management study. FIG. 74C depicts trial 1 of BUN management study. FIG. 74D depicts trial 2 of BUN management study.

FIG. 75, comprising FIGS. 75A and 75B, depicts plots of creatine removal. FIG. 75A depicts a plot of creatine removal using recirculated conventional dialysate (control). FIG. 75B depicts a plot of creatine removal using recirculated dialysate with an adsorbent (NAC).

FIG. 76 depicts a graph of potassium management: lowering concentration gradient and managing concentration gradient throughout the treatment.

FIG. 77 depicts a graph of hyperkalemia management: offering precision medicine using one dose of specific binders.

FIG. 78 depicts a graph of hyperkalemia management: offering precision medicine using specific binders in two doses in response to live measurements.

FIG. 79 depicts a series of SEM images of cross-linked polystyrene particles.

FIG. 80 depicts a series of SEM images of cross-linked polystyrene-Lokelma particles.

FIG. 81 depicts a series of SEM-EDS images of Chitosan-lanthanum composite

FIG. 82 depicts phosphate removal in porcine blood by porous chitosan particles and porous chitosan-Lanthanum particles.

FIG. 83 depicts image of syringe used for hemoperfusion experiment and table of results of 50 ml of blood passed through the porous chitosan/La (3.5 g).

### DETAILED DESCRIPTION

It is to be understood that the Figures and descriptions of the present invention have been simplified to illustrate elements that are relevant for a clear understanding of the present invention, while eliminating, for the purpose of clarity, many other elements used in fluid purification. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

As used herein, each of the following terms have the meanings associated with it as specified below. Unless defined otherwise, all technical and scientific terms used herein generally have the same meaning as commonly understood by one of ordinary skill in the art to which this invention belongs.

The articles “a” and “an” are used herein to refer to one or to more than one (i.e. to at least one) of the grammatical object of the article. By way of example, “an element” means one element or more than one element.

As used herein, the term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The terms “fluid” or “liquid medium” are used herein to refer to a substance which is in the form of a liquid at ambient temperature or room temperature. Non-limiting examples of fluid include: water, blood, blood serum, body fluids, dialysate, oils, milk, and any combination thereof.

The term “water” is used herein to preferably refers to water having a total organic carbon content of at least 4 ppm, or at least 5 ppm, or at least 10 ppm, or at least 50 ppm, or at least 100 ppm, or at least 500 ppm, or at least 1000 ppm. Alternatively, the term “water” may refer to wastewater from various sources, ocean or sea water, river water, etc. In addition, the term “purified water” as used herein refers to water having a total organic carbon content of less than 10 ppm, preferably less than 5 ppm, preferably less than 4 ppm, preferably less than 3 ppm, preferably less than 2 ppm, preferably less than 1 ppm.

The term “bodily fluid”, as used herein, can refer to a naturally occurring fluid from an animal, such as saliva, sputum, serum, plasma, blood, urine, mucus, gastric juices, pancreatic juices, semen, products of lactation or menstruation, tears, or lymph.

As used here, the term “blood” means whole blood and blood fractions, components, and products of blood, unless “whole blood” or a specific blood derivative, e.g., a blood fraction, component or product of blood is stated. The term “plasma” is used in its conventional sense to refer to the straw-colored/pale-yellow liquid component of blood composed of about 92% water, 7% proteins such as albumin, gamma globulin, anti-hemophilic factor, and other clotting factors, and 1% mineral salts, sugars, fats, hormones and vitamins.

The term “membrane” is used herein to refer to a film capable of performing separations. The separation may be absolute (i.e., non-permeable membrane), selective (i.e., semi-permeable membrane), or limited (i.e., permeable membrane).

The terms “filter” or “filter medium” are used herein to refer to any medium suitable for physical separation of liquids and solids. Filter presses generally comprise filter plates having a filter medium disposed thereon. Filter medium may include substrates having pores sized to exclude passage of solid particles, while allowing passage of smaller liquid molecules (e.g., filter cloths, membranes and the like). Filter medium also includes substrates comprising a plurality of particles, such that the particles serve as a physical barrier to the passage of other solid particles (e.g., diatomaceous earth and the like). Non-limiting examples of filter media include: PES (polyethersulfone) membranes, cellulose, cellulose acetate and regenerated cellulose membranes (i.e., typical paper filters), polypropylene membranes/cloth, Teflon and other fluoropolymer (hydrophilic and hydrophobic) membranes, glass fibers or fritted glass, other polymer membranes (e.g., polyester), metal mesh, charcoal, powdered activated carbon (PAC), graphite, graphene, graphene oxide, Molybdenum trioxide (MoO₃), manganese oxides (MnOₓ), manganese sulfides (MnSₓ), molybdenum oxides (MoOₓ), molybdenum sulfides (MoSₓ), silicon oxides (SiOₓ), silicon sulfides (SiSₓ), aluminum oxides (Al_{y}O_{z}), aluminum sulfides (Al_{y}S_{z}), boron oxides (B_{y}O_{z}), zeolites, tungsten diselenide (WSe₂), niobium diselenide (NbSe₂), boron nitride (BN), tungsten sulfide (WS₂), phosphorene (PR₃), tin (Sn), and transition metal di-chalcogenides.

The phrase “passing contaminated fluid through a membrane” is used herein to refer to a process whereby the contaminated fluid from an upstream source is brought into contact with an inner semi-permeable membrane, and preferably a pressure and/or stirring is applied to force the contaminated fluid through the inner semi-permeable membrane. The pressure may be a positive pressure, which is provided by, for example, a positive displacement pump that is located upstream of and fluidly connected to the membrane. Alternatively, the pressure may be a negative pressure, which is provided by, for example, a vacuum pump that is located downstream of and fluidly connected to the membrane.

The term “nanomaterial” is used herein to refers to a material having at least one dimension on the order of nanometers (e.g. between about 1 and 1000 nanometers). Nanomaterials include, but are not limited to, nanoparticles, nanocrystals, nanowires, nanorods, nanoplates, nanotubes and the like.

The terms “nanoparticle” or “nanocrystal” are used herein to refer to a particle having at least one diameter on the order of nanometers (e.g. between about 1 and 1000 nanometers).

The term “nanowire” is used herein to refer to a wire-like structure having at least one diameter on the order of nanometers (e.g. between about 1 and 1000 nanometers) and an aspect ratio greater than or equal to 10:1. The “aspect ratio” of a nanowire is the ratio of the actual length (L) of the nanowire to the diameter (D) of the nanowire.

The term “nanoplate” is used herein to refer to a plate-like structure having at least one dimension on the order of nanometers (e.g. between about 1 and 1000 nanometers) and an aspect ratio less than or equal to 1:5.

The term “nanotubes” is used herein to refer to cylindrical structures having at least one diameter on the order of nanometers (e.g. between about 1 and 1000 nanometers). Nanotubes generally have an aspect ratio greater than or equal to 10:1. Exemplary nanotubes include carbon nanotubes and silicon nanotubes.

The term “nanorod” is used herein to refer to a rod-like structure having at least one diameter on the order of nanometers (e.g. between about 1 and 1000 nanometers) and an aspect ratio less than 10:1.

The term “adsorbent” is used herein to refer to a substance which has the ability to condense or hold molecules of other substances on its surface or in its inner structure, an activity often referred as “adsorbing” or “absorbing”.

The term “organic” is used herein to refer to polymeric materials as well as small molecule organic materials and biological macromolecules (e.g., proteins, nucleic acids, etc.). For example, preferred small organic molecules range in size from approximately 10 Da up to about 5000 Da, more preferably up to 2000 Da, and most preferably up to about 1000 Da.

The term “inorganic” is used herein to refer to a substance comprising a metal element. Typically, an inorganic substance (e.g., nanowire, metal oxides, clay) includes one or more metals in its elemental state, or more preferably, a compound formed by a metal ion (Mⁿ⁺, wherein n is 1, 2, 3, 4, 5, 6 or 7) and an anion (X^{m-}, m is 1, 2, 3 or 4) which balance and neutralize the positive charges of the metal ion through electrostatic interactions. Non-limiting examples of inorganic compounds include oxides, hydroxides, oxyhydroxides, halides, nitrates, oxynitrates, sulfates, carbonates, oxycarbonates, phosphates, acetates, oxalates, and combinations thereof, of metal elements.

The term “salt” is used herein to refer to a compound comprising negative and positive ions. Salts are generally comprised of cations and counter ions or anions and counter ions.

The term “oxide” is used herein to refer to a metal compound comprising oxygen. Examples of oxides include, but are not limited to, metal oxides (MₓO_{y}), metal oxyhalides (MₓO_{y}X_{z}), metal oxynitrates (MₓO_{y}(NO₃)_{z}), metal phosphates (Mₓ(PO₄)_{y}), metal oxycarbonates (MₓO_{y}(CO₃)_{z}), metal carbonates, metal oxyhydroxides (MₓO_{y}(OH)_{z}) and the like, wherein x, y and z are numbers from 1 to 100.

The term “contaminant” or “contaminating agent” is used herein to refer to an impurity added to or incorporated within a contaminated liquid medium. A contaminant may comprise any organic compound, any inorganic compound, and any element from the periodic table.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to materials and processes for fluid purification such as in dialysis systems.

### Purification Materials

In one aspect, the present invention relates to a composition comprising porous particles of at least one carbon-based material.

In one embodiment, the porous particles have a generally spherical shape. In one embodiment, the porous particles do not have a spherical shape. In one embodiment, the porous particles include macrostructural features such as fins, wings, or surface defects at the macro, micro, or nano level. In one embodiment, the particles comprise wings and/or fins and micro and/or nanopores. In one embodiment, the size, porosity, density, and density of the particles can be tuned for a desired utility.

In one embodiment, the particles comprise an organic polymer. In one embodiment, the organic polymer comprises a natural polymer. In one embodiment, the organic polymer comprises a synthetic polymer. In one embodiment, the organic polymer comprises a semi-synthetic polymer. In one embodiment, the organic polymer embeds inorganic materials.

In one embodiment, the particles of the present invention comprise one type of polymer. In one embodiment, the particles comprise a mixture or copolymer of two or more types of polymers. In one embodiment, the copolymer, a random copolymer, a block copolymer, or a mixture of, and there is no limit on the number of types of polymers considered in the copolymer.

Exemplary polymers include, but are not limited to, polyethylene glycol; polypropylene glycol; polylactic acid; polyvinyl methyl ether; polyvinyl ethyl ether; polyvinyl alcohol; polyvinyl esters such as polyvinyl acetate and poly(vinyl cinnamate); polyvinylpyrrolidone; polyacrylics and polyacrylates such as polyhydroxypropyl acrylate, poly(methyl acrylate), poly(methyl methacrylate), polyacrylic acid; polyesters such as polyglycolide, polyglycolic acid, polylactic acid, polycaprolactone, polyhydroxy-alkanoate, polyhydroxybutyrate, polyethylene adipate, polybutylene succinate, poly(3-hydroxybutyrate-co-3-hydroxyvalerate), polyethylene terephthalate, polybutylene terephthalate, polytrimethylene terephthalate, polyethylene naphthalate, and Vectran™; cellulose; unsaturated polyesters; methyl cellulose; hydroxyethyl cellulose; hydroxypropyl methyl cellulose; hydroxypropyl cellulose; ethyl hydroxyethyl cellulose; hydrophobically-modified cellulose; epoxy resins such as aromatic epoxy resins, aliphatic epoxy resins, alicyclic epoxy resins, and heterocyclic epoxy resins; more specific examples of the epoxy resins include bisphenol A-type epoxy resins, bisphenol F-type epoxy resins, bisphenol AD-type epoxy resins, fluorine-containing epoxy resins, triglycidyl isocyanurate, alicyclic glycidyl ether-type epoxy resins, alicyclic glycidyl ester-type epoxy resins, and novolac-type epoxy resins; triacetate polymers such as cellulose triacetate; dextran; hydrophobically-modified dextran; agarose; low-gelling-temperature agarose; latex; pectin; polyvinyl chloride; polypropylene; polyethylene; polystyrene; styrene-butadiene copolymer; acrylonitrile butadiene styrene (ABS); poly(ethylenimine); polycarbonate; polyetherimide; poly(ethylene glycol) (N) monomethacrylate; methylmethacrylate; poly(ethylene glycol) (N) monomethyl ether monomethacrylate; nylon; nylon 6; nylon 6,6; chitosan; rayon; polytetrafluoroethylene (Teflon/PTFE); expanded polytetrafluoroethylene (e-PTFE), thermoplastic polyurethanes; polyacrylamides; polyacrylonitriles; polysulfones such as polysulfone, polyethersulfone, and polyphenylsulfone; and combinations thereof.

In one embodiment, the organic polymer comprises a natural polymer such as, but not limited to, cellulose, chitosan, albumin- and albumin-like polymeric composites, heparin and heparin-like composites, carrageenan, alginate, collagen, guar gum, agarose, carboxymethyl cellulose, hydroxyethyl cellulose, dextran, hyaluronic acid, pectin, alginic acid, agar, xanthan, collagen-glycosaminoglycan, collagen, pullulan, mannan, lignans such as Kraft lignin, lignosulfonate, alkali lignin, low sulfonate alkali lignin, Klason lignin, and acid hydrolysis lignin, and combinations thereof.

In one embodiment, the organic polymer includes one or more crosslinkers. Exemplary crosslinkers include glutaraldehyde, epichlorohydrin, sulfuric acid, hexamethylene diisocyante, aldehyde-dextran, aldehyde-pectin, aldehyde-starch, tripolyphosphate, 1,3,5-benzene tricarboxylic acid, terepthaladehdyde, genipin, carbodiimides, melamines, epoxides, isocyanates, aziridine, silanes, aziridines, polycarbodiimides, metal chelates, di- to poly-functional acrylates, trimethylolpropane, 1,2,6-hexanetriol, triethanol amine, polyphosphazene, glycoluril, benzoguanamine, urea, dihydrazides, diazides, divinylbenzene, polyamines such as triethylenetetraamine, polyamides, diacyl chlorides, dianhydrides, and combinations thereof.

In one embodiment, the polymer can be functionalized by grafting the functional groups to the polymer including amination, carboxylation, sulfonation, phosphorylation, hydroxylation, chelating groups, cationic or anionic groups, bioactive molecule immobilization, enzyme immobilization, introducing side chains or copolymerizing with monomers.

In one embodiment, the chitosan can be chemically modified including Carboxymethyl Chitosan (CMC), N,O-Carboxymethyl Chitosan, Chitosan-Graft-Polyethylene Glycol, Chitosan-Graft-Poly(lactic acid) or Poly(glycolic acid), Thiolated Chitosans, Quaternized Chitosan, Hydroxypropyl Chitosan, Phosphorylated Chitosan, Sulfated Chitosan. In another embodiment, chitosan polymer can be blended with fillers and other polymers to improve its mechanical properties. In another embodiment, chitosan polymer can be deacylated or acetylated to adjust the solubility, crystallinity, and mechanical properties.

In one embodiment, chitosan nanoparticles can be prepared using spray drying. Dissolved Chitosan in aqueous acetic acid can be passed through a nozzle using hot air temperatures to remove the solvent and form the nanoparticles.

In one embodiment, the particles have an average diameter between 1 nm and 10 mm. In one embodiment, the particles have an average diameter between 10 μm and 1 mm (1000 μm).

In some embodiments, the particles may have an average diameter of less than 1 μm, such as 100 nm to 1 μm.

In one embodiment the Brunauer-Emmet-Teller surface area of the particles is between 0.01 and 50 m²/g. In one embodiment, the Brunauer-Emmet-Teller surface area of the particles is between 1 and 35 m²/g. In one embodiment, the Brunauer-Emmet-Teller surface area of the particles is between 1 and 20 m²/g. In one embodiment, the Brunauer-Emmet-Teller surface area of the particles is between 1 and 10 m²/g. In one embodiment, the Brunauer-Emmet-Teller surface area of the particles is between land 5 m²/g. In one embodiment, the Brunauer-Emmet-Teller surface area of the particles is between 20 and 25 m²/g. In one embodiment, the Brunauer-Emmet-Teller surface area of the particles is between 25 and 30 m²/g. In one embodiment, the Brunauer-Emmet-Teller surface area of the particles is about 1 m²/g. In one embodiment, the Brunauer-Emmet-Teller surface area of the particles is about 21 m²/g. In one embodiment, the Brunauer-Emmet-Teller surface area of the particles is about 26 m²/g.

In one embodiment the Langmuir surface area of the particles is between 1 and 150 m²/g. In one embodiment, the Langmuir surface area of the particles is between 1 and 125 m²/g. In one embodiment, the Langmuir surface area of the particles is between 1 and 100 m²/g. In one embodiment, the Langmuir surface area of the particles is between 1 and 75 m²/g. In one embodiment, the Langmuir surface area of the particles is between 1 and 50 m²/g. In one embodiment, the Langmuir surface area of the particles is between 1 and 25 m²/g. In one embodiment, the Langmuir surface area of the particles is between 1 and 10 m²/g. In one embodiment, the Langmuir surface area of the particles is between 5 and 10 m²/g. In one embodiment, the Langmuir surface area of the particles is between 110 and 120 m²/g. In one embodiment, the Langmuir surface area of the particles is between 110 and 115 m²/g. In one embodiment, the Langmuir surface area of the particles is between 125 and 130 m²/g. In one embodiment, the Langmuir surface area of the particles is about 7 m²/g. In one embodiment, the Langmuir surface area of the particles is about 112 m²/g. In one embodiment, the Langmuir surface area of the particles is about 127 m²/g.

In one embodiment, the t-plot external surface area of the particles is between 1 and 100 m²/g. In one embodiment, the t-plot external surface area of the particles is between 1 and 85 m²/g. In one embodiment, the t-plot external surface area of the particles is between land 70 m²/g. In one embodiment, the t-plot external surface area of the particles is between 1 and 55 m²/g. In one embodiment, the t-plot external surface area of the particles is between 1 and 40 m²/g. In one embodiment, the t-plot external surface area of the particles is between 1 and 25 m²/g. In one embodiment, the t-plot external surface area of the particles is between 1 and 10 m²/g. In one embodiment, the t-plot external surface area of the particles is between 1 and 5 m²/g. In one embodiment, the t-plot external surface area of the particles is between 20 and 30 m²/g. In one embodiment, the t-plot external surface area of the particles is about 2 m²/g. In one embodiment, the t-plot external surface area of the particles is about 24 m²/g. In one embodiment, the t-plot external surface area of the particles is about 28 m²/g.

In one embodiment, the total pore volume of pores of the particles is between 1.0*10⁻³ and 0.5 cm³/g. In one embodiment, the total pore volume of pores of the particles is between 1.0*10⁻³ and 0.25. In one embodiment, the total pore volume of pores of the particles is between 1.0*10⁻³ and 0.15 cm³/g. In one embodiment, the total pore volume of pores of the particles is between 1.0*10⁻³ and 0.01 cm³/g. In one embodiment, the total pore volume of pores of the particles is between 0.05 and 0.25 cm³/g. In one embodiment, the total pore volume of pores of the particles is between 0.05 and 0.2 cm³/g. In one embodiment, the total pore volume of the pores of the particles is about 1.0*10⁻³ cm³/g. In one embodiment, the total pore volume of pores of the particles is about 0.1 cm³/g.

In one embodiment, the adsorption average pore diameter of the particles is between 1 and 100 nm. In one embodiment, the adsorption average pore diameter of the particles is between 1 and 85 nm. In one embodiment, the adsorption average pore diameter of the particles is between 1 and 70 nm. In one embodiment, the adsorption average pore diameter of the particles is between 1 and 55 nm. In one embodiment, the adsorption average pore diameter of the particles is between 1 and 40 nm. In one embodiment, the adsorption average pore diameter of the particles is between 1 and 25 nm. In one embodiment, the adsorption average pore diameter of the particles is between 1 and 15 nm. In one embodiment, the adsorption average pore diameter of the particles is between 5 and 10 nm. In one embodiment, the adsorption average pore diameter of the particles is between 15 and 20 nm. In one embodiment, the adsorption average pore diameter of the particles is between 25 and 30 nm. In one embodiment, the adsorption average pore diameter of the particles is about 7 nm. In one embodiment, the adsorption average pore diameter of the particles is about 17 nm. In one embodiment, the adsorption average pore diameter of the particles is about 29 nm.

In one embodiment, the Barett-Joyner-Halenda adsorption average pore diameter of the particles is between 0.01 and 1 nm. In one embodiment, the Barett-Joyner-Halenda adsorption average pore diameter of the particles is between 1 and 100 nm. In one embodiment, the Barett-Joyner-Halenda adsorption average pore diameter of the particles is between 1 and 85 nm. In one embodiment, the Barett-Joyner-Halenda adsorption average pore diameter of the particles is between 5 and 70 nm. In one embodiment, the Barett-Joyner-Halenda adsorption average pore diameter of the particles is between 5 and 50 nm. In one embodiment, the Barett-Joyner-Halenda adsorption average pore diameter of the particles is between 5 and 35 nm. In one embodiment, the Barett-Joyner-Halenda adsorption average pore diameter of the particles is between 5 and 15 nm. In one embodiment, the Barett-Joyner-Halenda adsorption average pore diameter of the particles is between 7 and 12 nm. In one embodiment, the Barett-Joyner-Halenda adsorption average pore diameter of the particles is between 15 and 25 nm. In one embodiment, the Barett-Joyner-Halenda adsorption average pore diameter of the particles is between 17 and 22 nm. In one embodiment, the Barett-Joyner-Halenda adsorption average pore diameter of the particles is between 25 and 35 nm. In one embodiment, the Barett-Joyner-Halenda adsorption average pore diameter of the particles is between 27 and 32 nm. In one embodiment, the Barett-Joyner-Halenda adsorption average pore diameter of the particles is about 10 nm. In one embodiment, the Barett-Joyner-Halenda adsorption average pore diameter of the particles is about 20 nm. In one embodiment, the Barett-Joyner-Halenda adsorption average pore diameter of the particles is about 30 nm.

In one embodiment, the deuterium-hydrogen adsorption average pore diameter of the particles is between 0.01 and 1 nm. In one embodiment, the deuterium-hydrogen adsorption average pore diameter of the particles is between 1 and 100 nm. In one embodiment, the deuterium-hydrogen adsorption average pore diameter of the particles is between 1 and 85 nm. In one embodiment, the deuterium-hydrogen adsorption average pore diameter of the particles is between 5 and 70 nm. In one embodiment, the deuterium-hydrogen adsorption average pore diameter of the particles is between 5 and 55 nm. In one embodiment, the deuterium-hydrogen adsorption average pore diameter of the particles is between 5 and 40 nm. In one embodiment, the deuterium-hydrogen adsorption average pore diameter of the particles is between 5 and 30 nm. In one embodiment, the deuterium-hydrogen adsorption average pore diameter of the particles is between 5 and 20 nm. In one embodiment, the deuterium-hydrogen adsorption average pore diameter of the particles is between 5 and 10 nm. In one embodiment, the deuterium-hydrogen adsorption average pore diameter of the particles is between 10 and 20. In one embodiment, the deuterium-hydrogen adsorption average pore diameter of the particles is between 15 and 20 nm. In one embodiment, the deuterium-hydrogen adsorption average pore diameter of the particles is about 6 nm. In one embodiment, the deuterium-hydrogen adsorption average pore diameter of the particles is about 7 nm. In one embodiment, the deuterium-hydrogen adsorption average pore diameter of the particles is about 16 nm.

In one embodiment, the deformation resistance of the particles is between 0.01 and 100 MPa. In one embodiment, the deformation resistance of the particles is between 0.1 and 85 MPa. In one embodiment, the deformation resistance of the particles is between 0.1 and 70 MPa. In one embodiment, the deformation resistance of the particles is between 0.1 and 55 MPa. In one embodiment, the deformation resistance of the particles is between 0.1 and 40.0 MPa. In one embodiment, the deformation resistance of the particles is between 0.1 and 30 MPa. In one embodiment, the deformation resistance of the particles is between 0.1 and 20 MPa. In one embodiment, the deformation resistance of the particles is between 0.1 and 15 MPa. In one embodiment, the deformation resistance of the particles is between 0.1 and 5 MPa. In one embodiment, the deformation resistance of the particles is between 0.1 and 2 MPa. In one embodiment, the deformation resistance of the particles is between 1 and 5 MPa. In one embodiment, the deformation resistance of the particles is between 5 and 10 MPa. In one embodiment, the deformation resistance of the particles is between 15 and 20 MPa. In one embodiment, the deformation resistance of the particles is about 1 MPa. In one embodiment, the deformation resistance of the particles is about 5 MPa. In one embodiment, the deformation resistance of the particles is about 10 MPa. In one embodiment, the deformation resistance of the particles is about 15 MPa. In one embodiment, the deformation resistance of the particles is about 50 MPa.

In one embodiment, the comprehensive strength of the particles is between 0.1 and 20 MPa. In one embodiment, the comprehensive strength of the particles is between 0.1 and 15 MPa. In one embodiment, the comprehensive strength of the particles is between 0.1 and 5 MPa. In one embodiment, the comprehensive strength of the particles is between 0.1 and 0.25 MPa. In one embodiment, the comprehensive strength of the particles is between 0.1 and 0.2 MPa. In one embodiment, the comprehensive strength of the particles is between 0.5 and 1.5 MPa. In one embodiment, the comprehensive strength of the particles is between 1.5 and 2.5 MPa. In one embodiment, the comprehensive strength of the particles is between 2.5 and 3.5 MPa. In one embodiment, the comprehensive strength of the particles is between 3.5 and 4.5 MPa. one embodiment, the comprehensive strength of the particles is about 0.1 MPa. In one embodiment, the comprehensive strength of the particles is about 1 MPa. In one embodiment, the comprehensive strength of the particles is about 2 MPa. In one embodiment, the comprehensive strength of the particles is about 3 MPa. In one embodiment, the comprehensive strength of the particles is about 4 MPa.

In one embodiment, the particles are solid. In one embodiment, the particles are porous. There is no particular limit to the pore size or the active surface area of the particles. In one embodiment, the particles are carbonized.

In one embodiment, the particles are core-shell particles comprising a core and a shell disposed over the core. In one embodiment, the core and the shell comprise the same material. In one embodiment, the core and the shell comprise separate materials. In one embodiment, the core and the shell have one or more materials in common. In one embodiment, the thickness of the shell is any value that is lower than the radius of the particle. In one embodiment, the ratio of shell to core as a function of radius or of weight ratio or molar ratio is any value between 99.99:0.01 to 0.01:99.99.

In one embodiment, the core and the shell of the core-shell particles have similar porosity. In one embodiment, the core and the shell have different porosities. In one embodiment, the shell of the core-shell particle is more porous than the core. In one embodiment, the core is more porous than the shell. In one embodiment, the core of the core-shell particle is solid (having minimal porosity) and the shell is porous.

In one embodiment, the core of the particle comprises an inorganic material. Exemplary inorganic materials include, but are not limited to, manganese oxides (MnOₓ), iron oxides (Fe_{y}O_{z}), manganese sulfides (MnSₓ), molybdenum oxides (MoOₓ) such as birnessite, molybdenum sulfides (MoSₓ), molybdenum trioxide (MoO₃), silicon oxides (SiOₓ), silicon sulfides (SiSₓ), aluminum oxides (Al_{y}O_{z}), aluminum sulfides (Al_{y}S_{z}), boron oxides (B_{y}O_{z}), zeolites, alumina, bauxite, silica, activated clays, bauxite, iron oxide or hydroxide, hydroxyapatite, zirconium Oxide, calcium alginate, metal-organic frameworks, layered double hydroxides, boron nitride and magnesium hydroxide. FDA approved bindery such as sodium zirconium cyclosilicate (Lokelma), and any combination thereof. In one embodiment, the core of the core-shell particle is magnetic.

In one embodiment, the particles comprise one or more functional groups. As used herein, “functional group” refers to a chemical moiety known to have affinity for a specific compound, salt, biomolecule, or any other potential component and/or contaminant in blood or any other solution. In one embodiment, the functional groups are covalently bound to the particles. In one embodiment, the functional groups are bound to the surface of the particles. In one embodiment, the functional groups are bound to the interior of the particles. In one embodiment, the functional groups are bound to the surface and the interior of the particles. Exemplary functional groups include active binders functional groups for removal of electrolytes, proteins, metabolites, or enzymes. In one embodiment, the functional groups comprise antibodies targeting specific proteins, metabolites, or enzymes. In one embodiment, the functional groups are selected for components of a fluid, such as blood. In one embodiment, the functional groups are biocompatible. In one embodiment, the functional groups are non-biocompatible. In one embodiment, functional groups that are non-biocompatible are functional groups that should not be placed in direct contact with blood. In one embodiment, the functional group comprises a polymer.

In one embodiment, the functional groups selectively and reversibly bind blood components. Exemplary blood components and drug intoxication include, but are not limited to, creatinine, blood urea nitrogen (BUN), albumin, phosphate, potassium, chloride ions, fluoride ions, magnesium, calcium, sodium, small molecules, hormones, amino acids, certain drugs and medications, cytokine, endotoxin and other uremic toxins such as γ-guanidinobutyric acid pseudouridine, 2-methoxyresorcinol, oxalate, α-n-acetylarginine orotic acid, indole-3-acetic acid, threitol, hyaluronic acid, hydroquinone, guanidinosuccinic acid, guanidine, benzylalcohol, indoxyl sulfate, interleukin-6, erythreitol, leptin, thymine, p-cresyl sulfate, hippuric acid, trimethylamine N-oxide, asymmetric dimethylarginine, endothelin, homocysteine, bilirubin, ammonia, nitrates and nitrites, interleukin-1 and Tumor Necrosis Factor alpha In one embodiment, the functional groups include chelating agents such as ethylenediaminetetraacetic acid (EDTA), cyclohexanediaminetetraacetic (CDTA), ethylene glycol tetraacetic acid (EGTA), citric acid, tartaric acid, polystyrene divinylbenzene copolymer and plant-based chelating agents.

In one embodiment, the functional groups selectively and reversibly bind to blood components that cause sepsis. Exemplary blood components include, but are not limited to, ammonia, phenylalanine, tyrosine, middle chain fatty acids, tryptophan and metabolites of it, endogenous benzodiazepines and other neuro-active substances, mercaptans, toxic bile acids, bilirubin, heavy metals and endogenous vasodilators.

In one embodiment, the functional groups include FDA-approved active ingredients with known binding activity. In one embodiment, the functional groups are capable of binding a specific compound, element, or salt. In one embodiment, the functional groups are capable of binding a specific blood component. In one embodiment, the functional group comprises a compound known to bind a specific blood component. In one embodiment, the particles comprise a compound known to bind a specific blood component, wherein the compound can be covalently bound to the particles, such as through a linker group or through a direct covalent bond, or the compounds are bound to the particles via electrostatic interactions or the compound may be bound via van der Waals interactions or other non-covalent interactions. In one embodiment, the compound has a known affinity for a specific blood component. In one embodiment, the FDA-approved active ingredients or functional groups comprise commercially available potassium binders. In one embodiment, the FDA-approved active ingredients or functional groups comprise one or more potassium binders. The binder can be cation exchange polymers or inorganic materials. Exemplary potassium binders include, but are not limited to, sodium zirconium cyclosilicate (Lokelma), patiromer, calcium polystyrene sulfonate, and sodium polystyrene sulfonate for potassium management. In one embodiment, the FDA-approved active ingredient or functional group comprises one or more phosphate binders. Exemplary phosphate binders include, but are not limited to, calcium acetate, sevelamer carbonate, sevelamer hydrochloride, aluminum hydroxide, calcium acetate/magnesium carbonate, calcium carbonate, colestilan, sucroferric oxyhydroxide, fermagate, ferric citrate and lanthanum salts, such as lanthanum carbonate, lanthanum fluoride, and lanthanum oxides, lanthanum hydroxide. Further exemplary binders include polymyxin b (Poly-Rx), deferoxamine (Desferal), edetate calcium disodium (Calcium Disodium Versenate), penicillamine (CUPRIMINE), dimercaptosuccinic acid (Chemet), rasburicase (Elitek), colestid (Colestipol), colesevelam (Welchol), and prevalite (Cholestyramine).

In one embodiment, the particles comprise nanoscale activated carbon. In one embodiment, nanoscale activated carbon particles have diameters between 1 and 10 nm. In one embodiment, nanoscale activated carbon particles have diameters between 10 and 25 nm. In one embodiment, nanoscale activated carbon particles have diameters between 25 and 50 nm. In one embodiment, the nanoscale activated carbon particles have diameters between 50 and 75 nm. In one embodiment, the nanoscale activated carbon particles have diameters between 75 and 100 nm. In one embodiment, the nanoscale activated carbon particles have diameters of at least 100 nm. In one embodiment, the nanoscale activated carbon particles have diameters of at least 200 nm In one embodiment, the particles comprise activated charcoal.

In one embodiment, the functional groups include enzymes and proteins that have a known activity or affinity. In one embodiment, enzymes and proteins are added as functional groups inside the beads. For example, urease catalyzes the hydrolysis of urea and ammonia and carbon dioxide are formed. Thus, in one embodiment, the functional group comprises urease. In one embodiment, the particles comprise urease. is added to manage urea. In one embodiment, the particles comprise a compound capable of binding middle and large blood molecules such as beta 2 microglobulin. Exemplary compounds include proteins such as collagen. In one embodiment, proteins and polymers are added to beads surface or inside the beads for removal of middle and large blood molecules ranging from 100 to 100,000 Daltons.

In one embodiment, the functional groups are pre-treated with their respective target moiety (the moiety for which the functional group has known affinity). In one embodiment, the target moiety is bound to the functional group.

In one embodiment, the particle is a core-shell particle comprising functional groups. In one embodiment, the functional groups may be bonded to any of the core, shell, or both. In one embodiment, the core and the shell comprise different functional groups. In one embodiment, the core comprises a non-biocompatible functional group. In one embodiment, the shell comprises a biocompatible material such as a biocompatible polymer. In one embodiment, the biocompatible shell prevents direct contact of blood with a non-biocompatible functional group within the core. “Thus, in one embodiment, non-biocompatible groups and components are shielded from direct blood contact with a biocompatible shell. In one embodiment, blood components reach the functional groups through pores.

In one embodiment, the particles comprise surface functional groups. In one embodiment, the surface functional groups are selected to affect solubility and/or dispersion in a solvent. In one embodiment, the particles comprise surface functional groups that impart a charge (e.g., zeta potential of greater than or less than 0) to the surface of the particle. In one embodiment, the surface functional groups comprise hydrophobic, hydrophilic, or lipophilic groups. In one embodiment, the surface functional groups comprise one or more surfactants. In one embodiment, the particles comprise more than one type of surface functional group.

In one embodiment, the composition further comprises a sol-gel material. In one embodiment, the sol-gel material is a swellable sol-gel material. In one embodiment, the sol-gel comprises a film forming precursor which forms the primary structure of the gel and the resulting coating. Exemplary film forming precursors include silicon containing precursors and titanium based precursors. The sol-gel material may further include alcohol and water as the solvent system, and either an inorganic or organic acid or base as a catalyst or accelerator. A combination of the aforementioned chemicals leads to formation of sol through hydrolysis and condensation reactions. Various coating techniques, including dip-coating, electrodeposition, spin coating, spray coating, roll coating, capillary coating, and curtain coating as examples, may be used to coat thin films of these sols onto the particles.

In one embodiment, the composition further comprises a solvent. In one embodiment, the particles are dispersed in the solvent. In one embodiment, the particles are suspended in the solvent. In one embodiment, the particles form a sediment in the solvent. In one embodiment, the solvent is an alcohol solvent. In one embodiment, the solvent is water. In one embodiment, the solvent is buffered water. In one embodiment, the solvent is saline. In one embodiment, the solvent is buffered saline.

In one embodiment, the composition further comprises a dispersant. In one embodiment, the dispersant is a surfactant. The surfactant is not particularly limited, and is, for example, an anionic surfactant, a cationic surfactant, a nonionic surfactant, or a block copolymer composed of a hydrophilic block and a hydrophobic block, such as, for example, a block copolymer composed of a polyacrylic acid block and a polyacrylic ester block, a block copolymer composed of a polyoxyethylene block and a polyacrylic ester block, or a block copolymer composed of a polyoxyethylene block and a polyoxypropylene block. In one embodiment, the composition comprises a polysorbate surfactant. Exemplary polysorbate surfactants include Polysorbate 20, Polysorbate 40, Polysorbate 60, Polysorbate 65, Polysorbate 80, and Polysorbate 85.

Exemplary anionic surfactants include fatty acid salts, sulfuric ester salts of higher alcohols, phosphoric ester salts of fatty alcohols, alkyl aryl sulfonate, and formalin condensates of naphthalene sulfonic acid salts. Exemplary cationic surfactants include alkyl primary amine salts, alkyl secondary amine salts, alkyl tertiary amine salts, alkyl quaternary ammonium salts, and pyridinium salts. Exemplary nonionic surfactants include polyoxyethylene alkyl ethers, polyoxyethylene alkyl phenylethers, polyoxyethylene alkyl esters, sorbitan alkyl esters, and polyoxyethylene sorbitan alkyl esters. Exemplary high molecular weight surfactants include partially-saponified polyvinyl alcohols, polyvinylpyrrolidone, starch, methylcellulose, carboxymethyl cellulose, hydroxyethyl cellulose, and partially-saponified polymatacrylic acid salts.

In one aspect, the present invention relates to any vessel comprising a composition disclosed herein. In one embodiment, the vessel comprises a syringe. In one embodiment, the syringe is a disposable plastic syringe. In one embodiment, the vessel contains the particles dispersed in, or suspended in, a solvent selected from the group consisting of water, buffered water, saline, and buffered saline.

In one embodiment, the vessel comprises a packed column. In one embodiment, the vessel comprises a cartridge. In one embodiment, the composition is placed in a vessel having an inlet and an outlet which are fluidly connected, such that any liquid entering via the inlet must pass through the composition en route to the outlet. In one embodiment, the composition is tightly packed using methods known in the art. In one embodiment, the composition is lightly packed. In one embodiment, the composition further comprises a material which disperses the particles in a solid or semi-solid matrix. In one embodiment, the composition is lightly packed. In one embodiment, the spacing (void volume of the column) is adjusted modulating surface features of the beads such as by controlling the size and density of fins and/or wings on the beads.

In one aspect, the present invention relates to a kit for performing any method described herein, the kit comprising a composition disclosed herein, a syringe, and instructions for performing the method using the provided components.

### Methods of Synthesizing Purification Materials

In one aspect, the present invention relates to a method of synthesizing a purification material comprising particles of at least one carbon-based material, the method comprising the steps of dispersing a polymer in a solvent to give a polymer solution; adding a foaming agent to the polymer solution; and treating the polymer solution with a base or an acid to form polymer beads. In one embodiment, the method further comprises the step of increasing the temperature of the polymer beads. In one embodiment, the increase in the pH of the polymer solution deprotonates the chitosan, reduces its solubility, and leads to formation of microsphere particles.

In one embodiment, the method further comprises the step of introducing porogen additives to induce pore formation. In one embodiment, the method further comprises the step of removing porogen additives to achieve a porous morphology. A suitable porogen additive may be a gas (carbon dioxide, nitrogen, or other inert gas), liquid (water, biological fluid), or solid. In one embodiment, a porogen additive comprises salts, sugars, or polysaccharides. In one embodiment, a porogen additive comprises natural or synthetic polymers, oligomers, or monomers. Exemplary porogen additive polymers include polyethylene glycol, poly(vinylpyrollidone), pullulan, poly(glycolide), poly(lactide), poly(lactide-co-glycolide). In one embodiment, the porogen additive comprises azocarbonamide, modified azodicarbonamide, p-toluenesulfonyl semicarbazide, p-toluenesulfonyl hydrazide, but are not limited to, hydrazide, p-toluenesulfonyl acetone hydrazide, 5-phenyltetrazole, sodium bicarbonate, or combinations thereof.

In one embodiment, the method further comprises the step of addition of surfactants. In one embodiment, the surfactant is a polysorbate surfactant. Exemplary polysorbate surfactants include Polysorbate 20 (Tween 20), Polysorbate 40, Polysorbate 60, Polysorbate 65, Polysorbate 80, and Polysorbate 85. In one embodiment, the surfactant is organosilicone surfactant. Exemplary organosilicone surfactants include 3-(2-methoxyethoxy) propyl-methyl-bis(trimethylsilyloxy) silane, a trisiloxane alkoxylate, and polyalkyleneoxide modified heptamethyltrisiloxane. In one embodiment, the surfactant is Poloxamers surfactant. In one embodiment, the surfactant is phospholipids surfactant. In one embodiment, the surfactant is polyethylene glycol surfactant. In one embodiment, the surfactant is zwitterionic polymers surfactant.

In one embodiment, the polymer can be any polymer material disclosed herein. In one embodiment, the polymer comprises moieties that are reactive in basic or acidic conditions. In one embodiment, the polymer comprises amine moieties. In one embodiment, the polymer comprises carboxylic acid moieties. In one embodiment, the polymer comprises phenolic moieties. In one embodiment, the polymer comprises chitosan.

In one embodiment, the step of dispersing a polymer in a solvent comprises the step of dissolving the polymer in the solvent. In one embodiment, the step of dispersing a polymer in the solvent comprises the step of suspending the polymer in the solvent. In one embodiment, the polymer does not fully dissolve in the solvent. In one embodiment, the solvent is a solvent known in the art to dissolve the polymer being used. In one embodiment, the solvent comprises water. In one embodiment, the solvent comprises at least one organic solvent. Exemplary organic solvents include, but are not limited to, hexane, heptane, pentane, diethyl ether, dichloromethane, 2-methylpentane, chloroform, benzene, toluene, methanol, ethanol, isopropanol, and ethyl acetate, olive oil, coconut oil, canola oil, sunflower oil, safflower oil, peanut oil, sesame oil, avocado oil, corn oil, flaxseed oil, grapeseed oil, soybean oil, walnut oil and almond oil. In one embodiment, the solvent comprises an acid. In one embodiment, the solvent comprises a dilute solution of acetic acid (1% to 5%). In one embodiment, the solvent comprises a dilute hydrochloric acid (1% to 5%). In one embodiment, the solvent comprises a dilute formic acid (1% to 5%). In one embodiment, the solvent comprises lactic acid. In one embodiment, the solvent comprises citric acid. In one embodiment, the solvent comprises succinic acid.

In one embodiment the, the method further comprises the step of dispersing a polymer in a water phase. In one embodiment, the water phrase comprises a salt such salt such as sodium chloride, potassium chloride, ammonium chloride, sodium bromide, sodium iodide, sodium sulfate, potassium bromide, potassium iodide, or combinations thereof. In one embodiment, the water phase comprises a polymer such as polyethylene glycol; polypropylene glycol; polylactic acid; polyvinyl methyl ether; polyvinyl ethyl ether; polyvinyl alcohol; polyvinyl esters such as polyvinyl acetate and poly(vinyl cinnamate); polyvinylpyrrolidone; polyacrylics and polyacrylates such as polyhydroxypropyl acrylate, poly(methyl acrylate), poly(methyl methacrylate), polyacrylic acid; polyesters such as polyglycolide, polyglycolic acid, polylactic acid, polycaprolactone, polyhydroxy-alkanoate, polyhydroxybutyrate, polyethylene adipate, polybutylene succinate, poly(3-hydroxybutyrate-co-3-hydroxyvalerate), polyethylene terephthalate, polybutylene terephthalate, polytrimethylene terephthalate, polyethylene naphthalate, and Vectran™; cellulose; unsaturated polyesters; methyl cellulose; hydroxyethyl cellulose; hydroxypropyl methyl cellulose; hydroxypropyl cellulose; ethyl hydroxyethyl cellulose; hydrophobically-modified cellulose; epoxy resins such as aromatic epoxy resins, aliphatic epoxy resins, alicyclic epoxy resins, and heterocyclic epoxy resins; more specific examples of the epoxy resins include bisphenol A-type epoxy resins, bisphenol F-type epoxy resins, bisphenol AD-type epoxy resins, fluorine-containing epoxy resins, triglycidyl isocyanurate, alicyclic glycidyl ether-type epoxy resins, alicyclic glycidyl ester-type epoxy resins, and novolac-type epoxy resins; triacetate polymers such as cellulose triacetate; dextran; hydrophobically-modified dextran; agarose; low-gelling-temperature agarose; latex; pectin; polyvinyl chloride; polypropylene; polyethylene; polystyrene; styrene-butadiene copolymer; acrylonitrile butadiene styrene (ABS); poly(ethylenimine); polycarbonate; polyetherimide; poly(ethylene glycol) (N) monomethacrylate; methylmethacrylate; poly(ethylene glycol) (N) monomethyl ether monomethacrylate; nylon; nylon 6; nylon 6,6; chitosan; rayon; polytetrafluoroethylene (Teflon/PTFE); expanded polytetrafluoroethylene (e-PTFE), thermoplastic polyurethanes; polyacrylamides; polyacrylonitriles; polysulfones such as polysulfone, polyethersulfone, and polyphenylsulfone; and combinations thereof.

In one embodiment, the solvent comprises a base. In one embodiment, the solution comprises a weak base or a strong base. In one embodiment, the solvent comprises a bicarbonate salt or a carbonate salt. In one embodiment, the solvent comprises an aqueous solution sodium hydroxide (1 to 10%). In one embodiment, the solvent comprises an aqueous solution potassium hydroxide (1 to 10%). In one embodiment, the solvent comprises an aqueous solution magnesium hydroxide (1 to 10%). In one embodiment, the solvent dissolves the polymer but does not depolymerize the polymer. In one embodiment, the beads are aged overnight. In one embodiment, the beads are rinsed with DI water to obtain a neutral pH.

In one embodiment, the method further comprises the step of adding a foaming agent. In one embodiment, the foaming agent comprises a blowing agent. Exemplary foaming agents include, but are not limited to azodicarboxamide, azodicarbonamide, modified azodicarbonamide, p-toluenesulfonyl semicarbazide, p-toluenesulfonyl hydrazide, hydrazide, p-toluenesulfonyl acetone hydrazide, 5-phenyltetrazole, and sodium bicarbonate. In one embodiment, the foaming agent comprises a hydrochlorofluorocarbon (HCFC), a hydrofluorocarbon (HFC), a hydrocarbon, liquid CO₂, a bicarbonate salt, isocyanate, or hydrazine. In one embodiment, the foaming agent comprises a salt that is removed from the polymer bead upon contact with the base or acid.

In one embodiment, the method further comprises the step of adding a rare-earth element. Exemplary rare-earth elements include Gadolinium, Neodymium, Scandium. Yttrium, Lanthanum, Praseodymium, Promethium, Samarium, Europium, Terbium, Dysprosium, Holmium, Erbium, Thulium, Ytterbium and Lutetium. In one embodiment, rare-earth elements are encapsulated in chitosan scaffolds. In one embodiment, chitosan-lanthanide scaffolds are used for device and sensor development, such as chitosan-Gadolinium composites.

particles (diameter 1 nm to 1+ mm) are made from the polymerization of different organic compounds, such as monomers of styrene, acrylics, sulfones, Methyl methacrylate, Ethyl acrylate, Butyl acrylate, Ethyl methacrylate, Butyl methacrylate, Hydroxyethyl methacrylate (HEMA), Glycidyl methacrylate, α-Methylstyrene, p-Methylstyrene, Vinyl toluene, N,N′-Methylenebisacrylamide, N-isopropylacrylamide, Methacrylic acid, Itaconic acid, Vinylidene chloride, Vinyl pyrrolidone, Vinyl fluoride, Vinyl ethers, glycidyl methacrylate, hydroxyethyl methacrylate, divinylbenzene diallyl dimethylammonium chloride, N,N′-methylene bis(acrylamide) and combinations thereof.

In one embodiment, the method further comprises the step of adding a radical initiator. Exemplary radical initiators include, but are not limited to, tert-Amyl peroxybenzoate, 4,4-Azobis(4-cyanovaleric acid), 1,1-Azobis(cyclohexanecarbonitrile), 2,2-Azobis(2-methylpropionitrile (AIBN), Benzoyl peroxide (BPO), 2,2-Bis(tert-butylperoxy) butane, 1,1-Bis(tert-butylperoxy)cyclohexane, 2,5-Bis(tert-butylperoxy)-2,5-dimethylhexane, 2,5-Bis(tert-Butylperoxy)-2,5-dimethyl-3-hexyne, Bis(1-(tert-butylperoxy)-1-methylethyl)benzene, 1,1-Bis(tert-butylperoxy)-3,3,5-trimethylcyclohexane, tert-Butyl hydroperoxide, tert-Butyl peracetate, tert-Butyl peroxide, tert-Butyl peroxybenzoate, tert-Butylperoxy isopropyl carbonate, Cumene hydroperoxide, Cyclohexanone peroxide, Dicumyl peroxide, Lauroyl peroxide, 2,4-Pentanedione peroxide, Peracetic acid, and Potassium persulfate, ammonium persulfate, sodium metabisulfite, sodium bisulfite, di-tert-butyl diperoxyphthalate.

In one embodiment, the microwave initiation method was used to synthesize particles. Microwaves with electromagnetic radiations in the frequency range of 300 MHz to 300 GHz are used to synthesize polymer particles.

In one embodiment, different stabilizing agents are used to prevent particle aggregation by providing a physical and/or electrostatic barrier around the particles including surfactant, polymers, copolymers, proteins, polypeptides, inorganic molecules, small molecules, phospholipids, and polysaccharides. In one embodiment, the step of suspending a polymer in a solvent further comprises the step of adding a functional group to the solvent. In one embodiment, the functional group is any functional group described herein. In one embodiment the functional group is covalently tethered to the polymer, such as through a direct bond or a divalent linking group. In one embodiment, the functional group is not directly bonded to the polymer. In one embodiment, the functional group is added to the solvent before the polymer is added. In one embodiment, the functional group is added to the solvent after the polymer is added.

In one embodiment, the step of treating the polymer solution with a base or an acid to form polymer beads comprises the step of dropping small amounts of the polymer solution in a vessel comprising the base or the acid. In one embodiment, the size of the drops can be controlled such as by manipulating the size or volume of a buret/dispenser. In one embodiment, the size of the drops can be controlled by manipulating the concentration or density of the polymer solution. In one embodiment, the polymer in the polymer solution is forced out of solution by the action of the acid or the base, thereby forming insoluble beads. In one embodiment, the pH of the acid or the base can be selected to counter the pH of the polymer solution. For example, if the polymer is dispersed in a basic solution, the polymer solution can be dropped into an acidic solution. As another example, if the polymer is dispersed in an acidic solution, the polymer solution can be dropped into a basic solution.

In one embodiment, the solution comprising the base or the acid further comprises one or more functional groups. In one embodiment, the functional groups are incorporated into the beads when the polymer is forced out of solution.

In one embodiment, the step of treating the polymer solution with a base or an acid to form polymer beads further comprises the step of activating a foaming agent in the polymer solution. In one embodiment, the base or the acid cases the foaming agent to disperse rapidly, thereby forming pores in the polymer beads. In one embodiment, the action of the foaming agent further forms other surface features on the polymer beads, such as wings or fins.

In one embodiment, the method further comprises the step of treating the polymer beads with a crosslinking agent. In one embodiment the crosslinking agent is a polyfunctional polymer. Exemplary crosslinking agents include glutaraldehyde, epichlorohydrin, sulfiric acid, hexamethylene diisocyante, aldehyde-dextran, aldehyde-pectin, aldehyde-starch, tripolyphosphate, 1,3,5-benzene tricarboxylic acid, terepthaladehdyde, genipin, carbodiimides, melamines, epoxides, isocyanates, aziridine, silanes, aziridines, polycarbodiimides, metal chelates, di- to poly-functional acrylates, trimethylolpropane, 1,2,6-hexanetriol, triethanol amine, polyphosphazene, glycoluril, benzoguanamine, urea, dihydrazides, diazides, polyamines such as triethylenetetraamine, polyamides, diacyl chlorides, dianhydrides, and combinations thereof. In one embodiment, the crosslinking agent can decrease the diameter of the resulting particle and can improve uptake of contaminants.

In one embodiment, the method further comprises the step of heating the polymer beads. In one embodiment, increasing the temperature of the polymer beads can cause release of volatile components of the polymer solution trapped within the polymer beads, leaving pores and various surface features in their wake. In one embodiment, the method further comprises the step of treating the polymer bead with an additional chemical agent, which reacts a foaming agent in the polymer bead and leaves pores and/or other surface features in the beads. In one embodiment, the method comprises the further step of carbonizing the polymer beads.

In another aspect, the present invention relates to a method of synthesizing porous polymer beads, the method comprising the steps of providing a mixture of one or more polymer material and a blowing agent, and irradiating the mixture with microwaves to produce porous polymer beads. In one embodiment, the polymer material can be any polymer material disclosed herein. In one embodiment, the polymer material comprises a hydrocarbon polymer. In one embodiment, the polymer comprises polystyrene, divinylbenzene, or a mixture thereof. In one embodiment, the blowing agent comprises azodicarboxamide (azodicarbonamide), a hydrochlorofluorocarbon (HCFC), a hydrofluorocarbon (HFC), a hydrocarbon, liquid CO₂, a bicarbonate salt, isocyanate, or hydrazine. In one embodiment, the method comprises the further step of carbonizing the polymer beads.

In one embodiment, the step of providing a mixture of a mixture of one or more polymer material and a blowing agent further comprises the step of adding a functional group to the polymer material. In one embodiment, the functional group is covalently bonded to the polymer material. In one embodiment, the functional group is linked to the polymer material through a divalent liking group. In one embodiment, the functional group is not covalently linked to the polymer material.

In one embodiment, the porous polymer beads produced by this method comprise gel-like porous spheres. In one embodiment, the porous polymer beads produced by this method comprise nanoparticles and/or crystals bound to swellable gel-like polymers. In one embodiment, the porous polymer beads produced by this method comprise nanoparticles or microparticles, which optionally comprise functional groups, linked to a swellable gel-like polymer tail. In one embodiment, the functional group comprises sodium zirconium cyclosilicate (Lokelma).

In one embodiment, the porous composition produced by this method can be used to prepare porous structures such as film or bulk materials. In one embodiment, the porous composition produced by this method can be used to prepare a purification material comprising porous particles for a filtration cartridge.

### Methods of Dispersal

In one aspect, the present invention relates to a method of dispersing a medical composition in a filtration unit. In one embodiment, the method comprises the steps of providing a vessel comprising the composition, securing the vessel to the filtration unit, and depositing the composition in to the filtration unit; wherein the medical composition comprises particles of at least one carbon-based material; wherein the filtration unit comprises a filtration membrane having hollow fiber pores; and wherein the average diameter of the particles is larger than the hollow fiber pores of the filtration unit.

In one embodiment, the vessel is a syringe. In one embodiment the vessel is a syringe comprising a Luer lock. In one embodiment, the step of attaching the vessel to the filtration unit comprises the step of attaching a syringe to the filtration unit and injecting the medical composition into the filtration unit.

In one embodiment, the filtration unit is a dialyzer used for the dialysis of blood and blood products (haemodialysis, haemofiltration or haemodiafiltration) In one embodiment, the filtration unit is a commercial dialysis filter such as Polyflux® Revaclear, Polyflux®, Optiflux®, Polysulfone®, Helixone® or FX class dialysers. In one embodiment the dialyzer is powered by a pump. In one embodiment, the pump is a peristaltic pump. In one embodiment, the pump is a hose pump. In one embodiment, the pump is a tube pump. In one embodiment the peristaltic pump is set to a desired flow rate.

In one embodiment, the injection of saline solution, with the particles, into a commercial dialyzer (filtration unit) allows enhanced and controlled mass transfer between patient's blood and dialysate solution, and provides functions in a hollow fiber membrane.

In one aspect, the present invention relates to a method of purifying a fluid using the composition described herein. In one embodiment, the fluid comprises water. In one embodiment, the fluid comprises an emulsion. In one embodiment, the fluid comprises a drinking fluid. In one embodiment, the fluid comprises a beverage. In one embodiment, the fluid comprises a bodily fluid. In one embodiment, the fluid comprises blood. In one embodiment, the fluid comprises blood serum. In one embodiment, the fluid comprises an oil. In one embodiment, the fluid comprises milk. In one embodiment, the fluid comprises an alcohol. In one embodiment, the fluid comprises a solvent. In one embodiment, the fluid comprises an organic solvent. In various embodiments, the fluid may comprise any combination of water, drinking fluids, beverages, blood, blood serum, oils, milk, alcohols, solvents, and organic solvents.

In one aspect, the present invention relates to a method of providing essential electrolytes, vitamins, proteins, and/or enzymes to a subject in need thereof. In one embodiment, the method comprises the step of providing a composition comprising particles having functional groups which selectively and reversibly bind to the essential electrolyte, vitamin, protein, and/or enzyme; contacting the composition with a solution comprising the essential electrolyte, vitamin, protein, and/or enzyme so that the functional groups become saturated with the essential electrolyte, vitamin, protein, and/or enzyme; contacting the saturated composition with a the blood of the subject or with a dialysate which contacts the blood of the subject, such as through a filtration membrane; and releasing the essential electrolyte, vitamin, protein, and/or enzyme into the blood or dialysate such that the essential electrolyte, vitamin, protein, and/or enzyme enters the body of the subject. In one embodiment, the step of releasing the essential electrolyte, vitamin, protein, and/or enzyme comprises the step of exposing the particles to conditions which effect the release of the essential electrolyte, vitamin, protein, and/or enzyme. In one embodiment, said conditions may include varying the temperature or pressure in the system or may include treating the composition with additional agents which effect the release of the essential electrolyte, vitamin, protein, and/or enzyme. In one embodiment, the saturated functional groups slowly release the essential electrolyte, vitamin, protein, and/or enzyme.

### Dialysis Methods

In one aspect, the present invention relates to a method of dialyzing the blood of a subject in need thereof with a dialysis unit comprising a composition disclosed herein. In one embodiment, the composition is loaded into the dialysis unit prior to the initiation of the dialysis procedure. In one embodiment, the composition comprises dialysate. In one embodiment, during the dialysis procedure, the dialysate is recirculated. In one embodiment, the dialysate is not recirculated. In one embodiment, the composition used in the dialysis unit comprises nanoparticle adsorbents. In one embodiment, the composition used in the dialysis unit is a mixture of nanoparticle adsorbents and dialysate. Dialysis methods that can be enhanced using the methods described herein include, but are not limited to, hemodialysis, peritoneal dialysis, continuous renal replacement therapy, hemoperfusion, liver dialysis, and sepsis dialysis, continuous ambulatory peritoneal dialysis (CAPD), continuous cyclic peritoneal dialysis (CCPD), and intermittent peritoneal dialysis (IPD).

In one embodiment, a packed column of the composition is provided. In one embodiment, the blood of the subject in need of dialysis is directly contacted with the packed column comprising the composition, and the dialysate is not contacted with the composition. In one embodiment, the dialysate is not recirculated. In one embodiment, the dialysate is directly contacted with the packed column comprising the composition, and the blood is not contacted with the composition. In one embodiment, the dialysate is partially recirculated.

In one aspect, the present invention relates to a dialysis system comprising dialyzer unit having a blood inlet, a blood outlet, and dialysate inlet, a dialysate outlet; wherein the blood inlet and blood outlet are fluidly connected and the dialysate inlet and the dialysate outlet are fluidly connected; a dialysate containment vessel which is fluidly connected to the dialysate inlet port via a filtration line and a bypass line; wherein the bypass line includes a bypass valve between the dialyzer unit and the dialysate vessel; and wherein the filtration line includes a filtration cartridge fluidly connected to the dialysate containment vessel and the dialysate inlet and a valve between the purification cartridge and the dialysate containment vessel, such that flow from the dialysate containment vessel can be directed through the purification cartridge, through the bypass vessel, or through both the purification cartridge and the bypass vessel.

In one aspect, the present invention relates to a method of using the dialysis system disclosed herein. In one embodiment, the method comprises the steps of adding a composition described herein to a dialyzer unit; providing a dialysate solution; passing blood and the dialysate solution through the dialyzer unit; and removing a blood component from the blood.

In one embodiment, the step of providing a dialysate solution comprises the step of adding the blood component to the dialysate solution, such that blood component concentration of the dialysate solution is higher than that of a standard dialysate solution but lower than the blood component level of the blood to be treated. In one embodiment, the step of passing blood and the dialysate solution through the dialyzer unit comprises the step of: passing the dialysate through the bypass line of the dialysis system and through the dialyzer until the blood component level of the blood and dialysate have equilibrated; and then directing at least a portion of the dialysate solution through the purification cartridge, wherein the purification cartridge comprises a composition described herein that comprises a functional group to remove the blood component from the dialysate solution. In one embodiment, the dialysis system disclosed herein can effectively decrease the blood component gradient between the blood and the dialysate solution, thereby decreasing undesirable effects due to high blood component gradient.

In one embodiment, FDA-approved active ingredients are added to the dialysate. In one embodiment, potassium binders are added to the dialysate. In one embodiment, phosphate binders are added to the dialysate. Exemplary FDA-approved active ingredients include, but not limited to, sodium zirconium cyclosilicate (Lokelma), patiromer, calcium polystyrene sulfonate, and sodium polystyrene sulfonate, calcium acetate, sevelamer carbonate, aluminum hydroxide, calcium acetate/magnesium carbonate, calcium carbonate, colestilan, sucroferric oxyhydroxide, fermagate and lanthanum, such as lanthanum carbonate. Polymyxin B is FDA-approved for the treatment of acute infections.

The mass of added adsorbents varies depending on adsorbent type, target blood toxin, and expected removal rate. In one embodiment, 0.5 to 5 g/l of adsorbents are added to the dialysate. In one embodiment, 5 to 10 g/l of adsorbents are added to the dialysate. In one embodiment, 10 to 15 g/l of adsorbents are added to the dialysate. In one embodiment, 15 to 20 g/l of adsorbents are added to the dialysate. In one embodiment, 20 to 25 g/l of adsorbents are added to the dialysate. In one embodiment, at least 25 g/l of adsorbents are added to the dialysate. In one embodiment, 20 to 25 g/l of adsorbents are added to the dialysate. In one embodiment, at least 30 g/l of adsorbents are added to the dialysate.

In one embodiment, blood flow rates are adjusted to meet requirements for toxin clearance. In one embodiment, dialysate rates are adjusted to meet requirements for toxin clearance. In one embodiment, blood flow rate is between 5 and 100 mL/min. In one embodiment, blood flow rate is between 100 and 500 mL/min. In one embodiment, blood flow rate is between 500 and 1000 mL/min. In one embodiment, blood flow rate is up to 2000 mL/min. In one embodiment, dialysate flow rate is between 5 and 100 mL/min. In one embodiment, dialysate flow rate is between 100 and 500 mL/min. In one embodiment, dialysate flow rate is between 500 and 1000 mL/min. In one embodiment, dialysate flow rate is up to 2000 mL/min.

In one aspect, the present invention relates to a method of embedding active ingredients inside the porous biocompatible polymers and particles, to improve the biocompatibility and release of the particles and molecules to the solution in a porous structure. In one embodiment, the active ingredient is bound to a binding moiety in the porous particle. In one embodiment, the active ingredient is adsorbed onto a surface of the porous particle. In one embodiment, the porous particle further comprises a biocompatible polymer. In one embodiment, the surface of the porous particle comprises the biocompatible polymer. In one embodiment, the biocompatible polymer improves the biocompatibility of the porous particle. In one embodiment, the biocompatible polymer improves the release of the active ingredient from the porous particle. In one embodiment, upon exposure to a fluid such as a bodily fluid, the active ingredient bound to the porous particle is released to the fluid.

In one aspect, the present invention relates to a method of embedding and adding functional materials to a fiber filter polymer during a fiber preparation and can be used for a filtration cartridge. In one embodiment, the functional material comprises a porous particle described herein. In one embodiment, the porous particle is added to the fiber during the manufacture of a filtration cartridge comprising said fiber. Similarly, the present invention relates to a fiber filter, such as one present in a filtration filter, in which the porous particle described herein is bound to the fiber filter.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the materials, devices, and kits of the present invention and practice the claimed methods. The following working examples therefore, specifically point out exemplary embodiments of the present invention, and are not to be construed as limiting in any way the remainder of the disclosure.

The significance of the invention and claims is the synthesis and use of engineered materials at nano- and micrometer scales that are specific for fluid purification and drug delivery. Here, a combination of biocompatible hydrophobic, hydrophilic, lipophilic materials is used to synthesize scaffolds to deliver non-biocompatible and biocompatible hydrophobic and hydrophilic functional groups.

In one embodiment, the beads have wings/fins with macro pores to increase the spacing between the beads packed in column or cartridge and improve mass transfer between fluids and functional groups.

In one embodiment, functional group and active ingredients are added to beads surface. Beads contain functional groups on their surfaces and inside their shells. Active particles can function as adsorbents, slow-releasing desorbents, or catalyst.

In one embodiment, the beads carry proteins and enzymes. Proteins and enzyme can be released to fluid slowly or the reactions can take place inside the scaffolds. Dialysis methods that can be enhanced using the methods described herein include, but are not limited to, hemodialysis, peritoneal dialysis, continuous renal replacement therapy, hemoperfusion, liver dialysis, and sepsis dialysis, continuous ambulatory peritoneal dialysis (CAPD), continuous cyclic peritoneal dialysis (CCPD), and intermittent peritoneal dialysis (IPD).

### Example 1: Particles for Fluid Purification

In this novel approach, particles are injected to a commercial dialyzer, such as Fresenius Optiflux, prior to a hemodialysis treatment. The objective is to 1) add adsorbents for rapid and controlled removal of blood uremic toxins, proteins, metabolites, enzymes, pathogens, and electrolytes 2) add desorbents to slowly release proteins, enzymes, vitamins, minerals, and electrolytes. The superior advantage of this novel method is that it provides numerous functions to commercially available dialyzers by facile addition of particles.

In one embodiment, particles (diameter 10 μm to 1+ mm) are made of carbon-based materials, polymers, and/or copolymers. Polymers are the scaffolds of the spheres. In other embodiments scaffolds (polymers) are crossed linked, and in other embodiments scaffolds have binders and functional group specific to adsorbing or deterring blood proteins, metabolites, enzymes, pathogens, and electrolytes.

Alternatively, spheres could release essential electrolytes, vitamins, proteins, and enzymes to a dialysate solution and eventually patient's blood in a dialyzer during a hemodialysis treatment. In addition, spheres' surfaces could be functionalized to deter blood's essential components to retain them in blood.

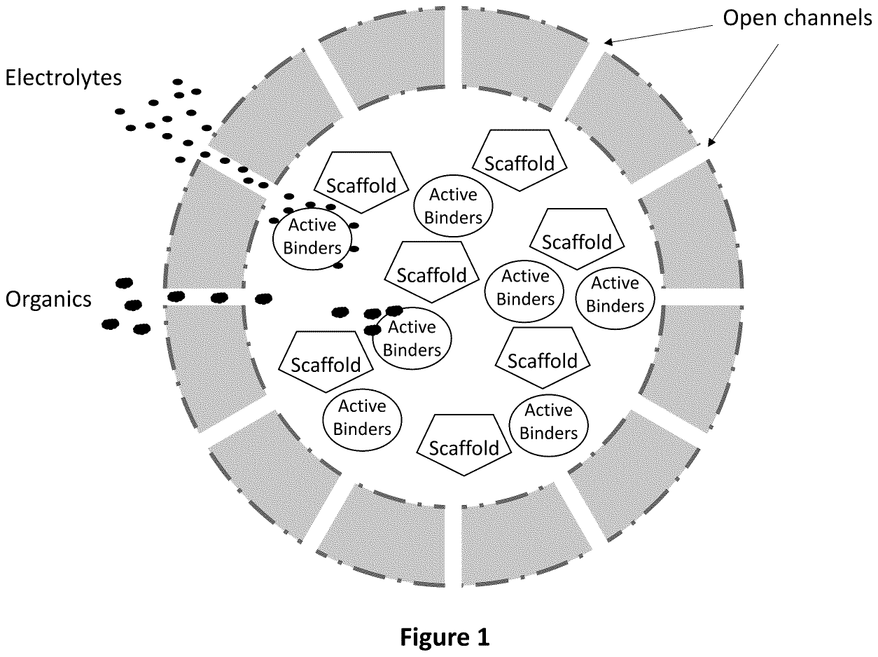

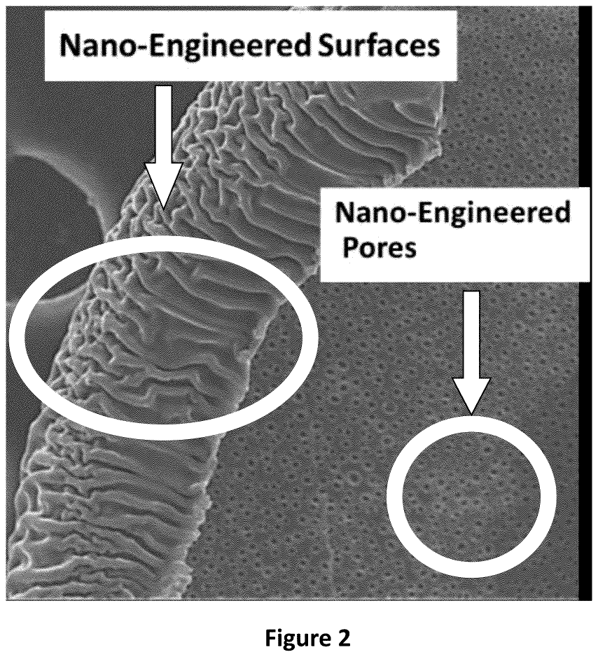

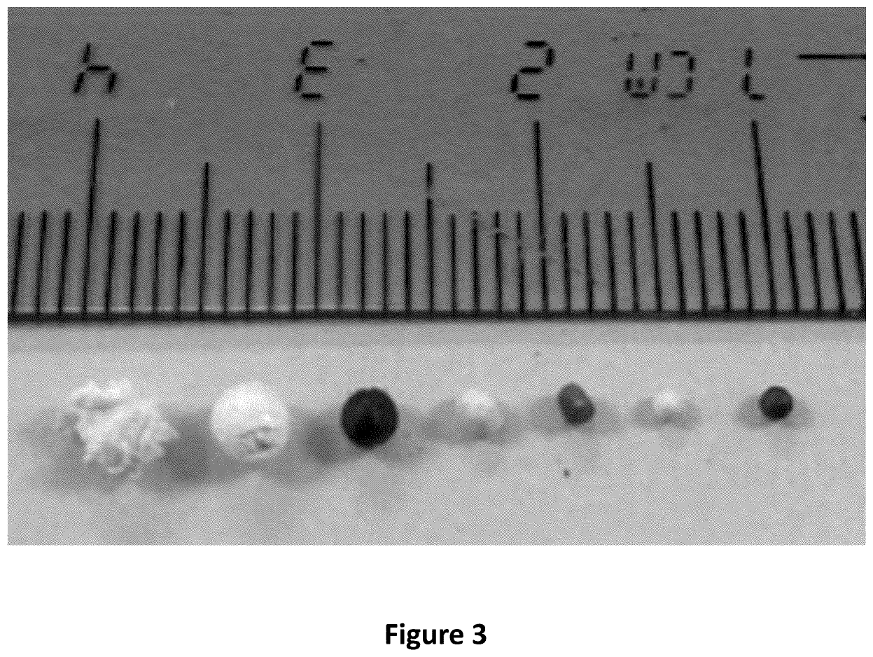

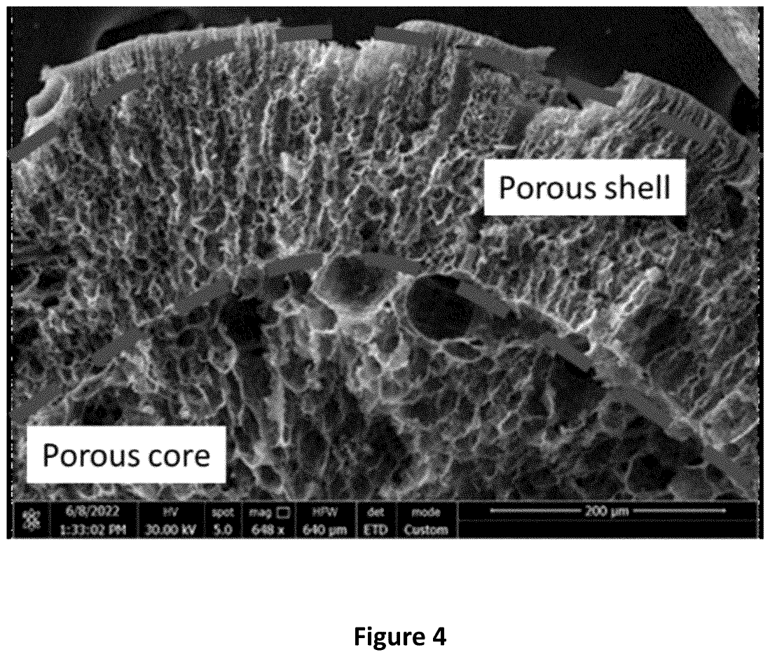

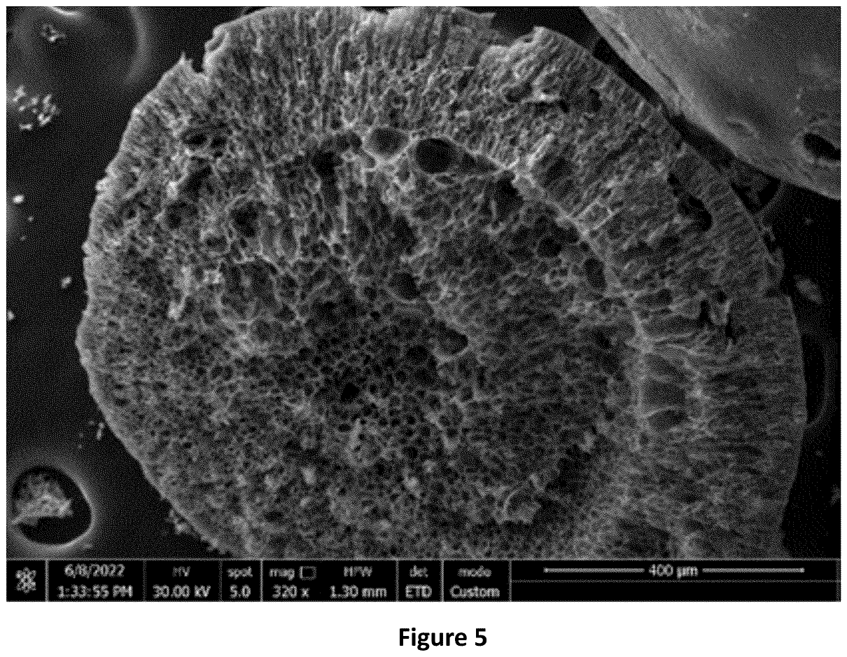

Particles (diameter 10 μm to 1+ mm) are made from chitosan, cellulose, lignin polymers or modified/functionalized polymers. In one embodiment polymers are crosslinked. In another embodiment, polymers can be used for the removal of electrolytes, proteins, metabolites, or enzymes. In another embodiment, the prepared polymers can be embedded in other polymers to embed and improve the biocompatibility of the polymers. In another embodiment, polymers can be blended with heparin mimicking polymers to improve biocompability. In another embodiment, the polymers carry active binders or functional groups for removal of electrolytes, proteins, metabolites, or enzymes. Different polymers, copolymers, and active binders are used. FIG. 1 depicts a conceptual design of the core-shell particles. FIG. 2 depicts a cross-sectional view of a particles under scanning electron microscopy (SEM), and FIG. 1 depicts its conceptual design. An alternative design concept is also proposed where the porous shell contains a porous core. A few examples of particles (resins) are shown in FIG. 3. FIGS. 4 and 5 are SEM images of a chitosan-based porous resin.

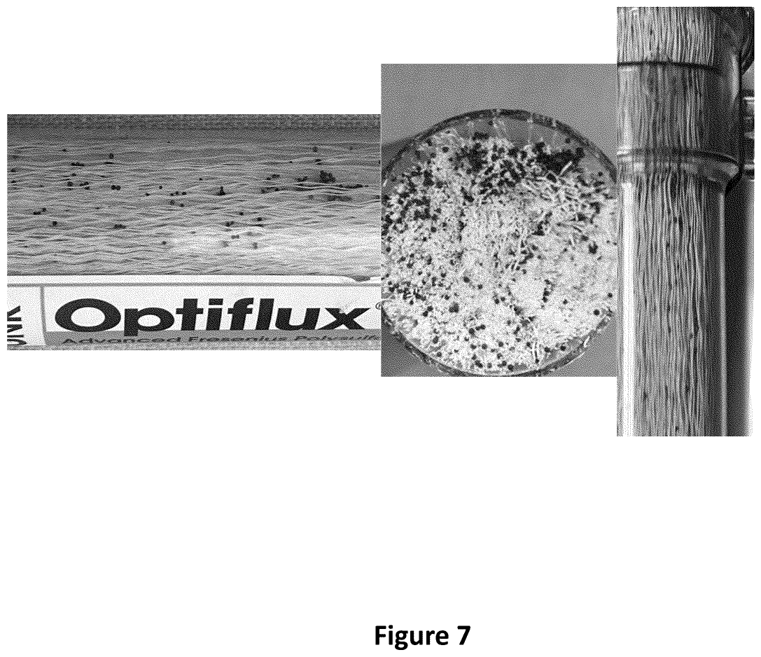

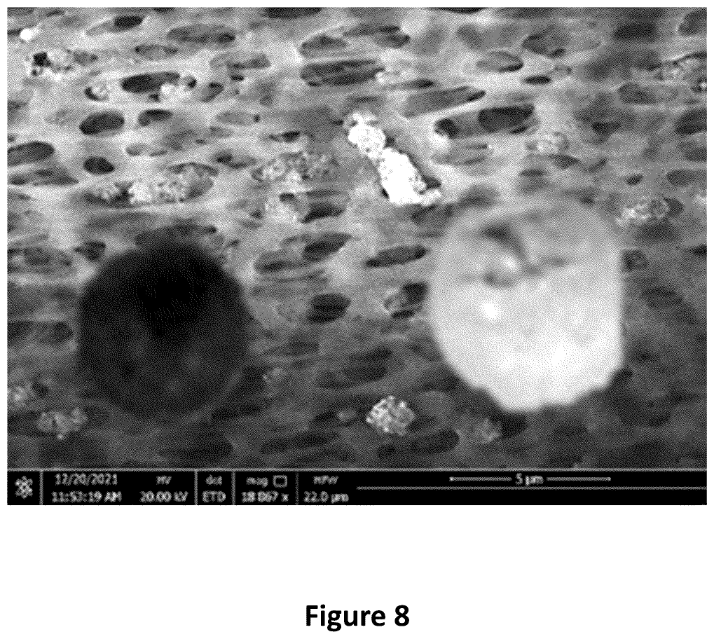

Process for the use of resin particles in a dialyzer (FIG. 6):
1—Dialyzers (membrane filtration unit for hemodialysis) are primed with saline solutions prior to use. This is a common practice in hemodialysis.
2—Particles are delivered in a large syringe containing saline solution.
3—The saline solution containing the particles is injected to a dialyzer before a treatment.
4—The injection of saline solution, with the particles, into a commercial dialyzer (filtration unit) allows enhanced and controlled mass transfer between patient's blood and dialysate solution, and provides functions in a hollow fiber membrane. Photographs of particles in an Optiflux (FreseniusF160NR) dialyzer after injection is presented in FIG. 7.
5—Particles are larger than hollow fiber membrane pores. As shown in FIG. 8, hollow fiber membranes are made of multiple layers of porous sheets. Added particles are considerably larger than pore size and thus cannot enter patient's blood.

The addition of resins as particles into commercially available dialyzers improve and control mass transfer between patients' blood and dialysate. The majority of the current research and development (R&D) efforts and the literature is mainly focused on material development and enhancement of hollow fiber membranes. This invention can be applied to commercially available dialyzers but enhance and control mass transfer by addition of nano-engineered particles specific to patients' need.

In one embodiment, this approach could be used to deliver FDA approved binders and pharmaceuticals into a dialyzer. Table 1, below, provides examples of FDA-approved phosphate and potassium binders that could be used in the new approach.

TABLE 1
Examples of FDA approved active ingredients that can be delivered
to commercially available dialyzers using this approach

| Active Ingredients | | |
|---|---|---|
| Brand | Compound | Pharm Co. |

Potassium (K+) Binders

| | | |
|---|---|---|
| Lokelma | Sodium zirconium | Astra Zeneca |
| | cyclosilicate | |
| Veltassa | Patiromer | Vifor Pharma |
| Kalexate, Kayexalate, | Sodium polystyrene | Various |
| Kione | sulfonate | |

Phosphate (PO₄³⁻) Binders

| | | |
|---|---|---|
| Phoslo | Calcium acetate | Amneal Pharmaceuticals, |
| | | Fresenius |
| Sevelamer, Renvela, | Sevelamer carbonate | Various |
| Renagel | | |
| Fosrenol | Lanthanum carbonate | Shire Pharmaceuticals Group |

Heavy Metal binders

| | | |
|---|---|---|
| Calcium Disodium | Edetate Calcium | Global Calcium |
| Versenate | Disodium | |
| Desferal | Deferoxamine | Novartis |
| CUPRIMINE | Penicillamine | Merck |
| Chemet | Dimercaptosuccinic acid | Sanofi |

Endotoxin Binder

| | | |
|---|---|---|
| Poly-Rx | Polymyxin B | Toray |

Metabolize uric acid

| | | |
|---|---|---|
| Elitek | Rasburicase | elitekpro |

Bile acids Binder

| | | |
|---|---|---|
| Colestipol | Colestid | Pfizer |
| Welchol | Colesevelam | Cosette Pharmaceuticals |
| Cholestyramine | Prevalite | Upsher-Smith Laboratories |

This approach simply requires dialysis nurses to inject particles into a dialyzer as part of priming the dialyzer (FIG. 6).

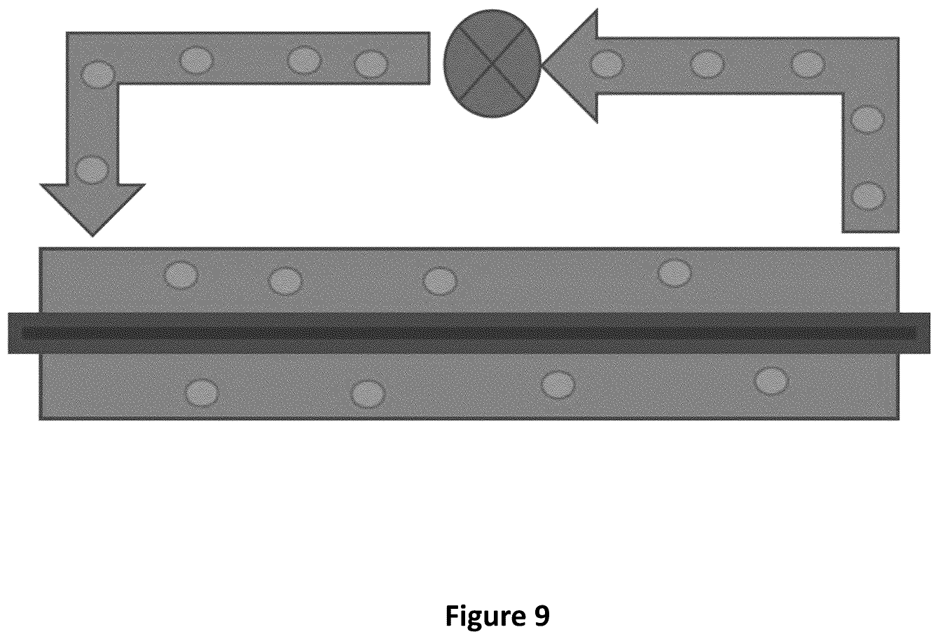

The particles described here can be used in any number of dialysis systems. For example, the particles can be used in a nano-slurry system (FIG. 9), in which nano adsorbents are added in the dialysate and the dialysate (nano-slurry) is recirculated.

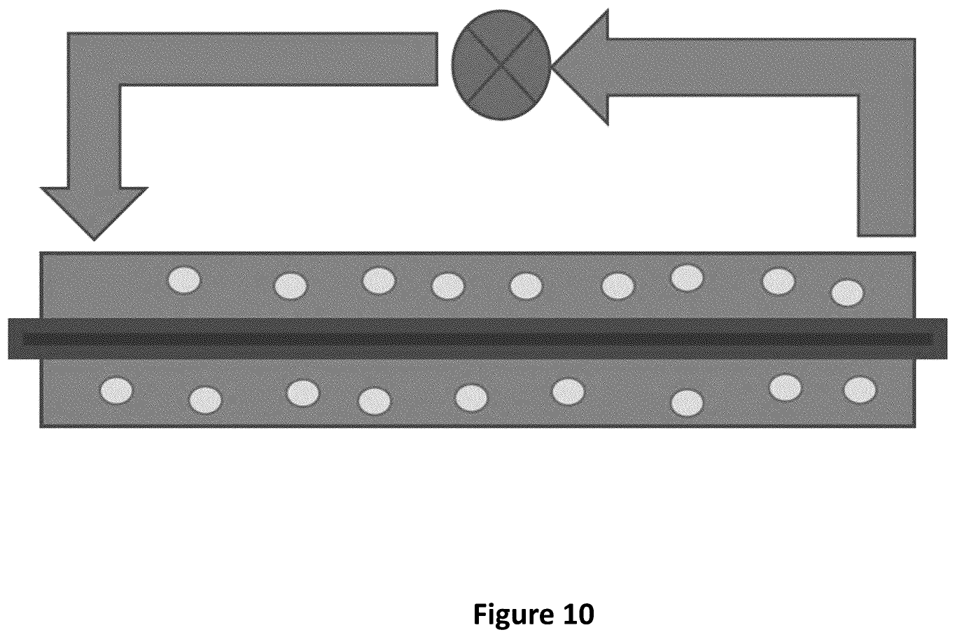

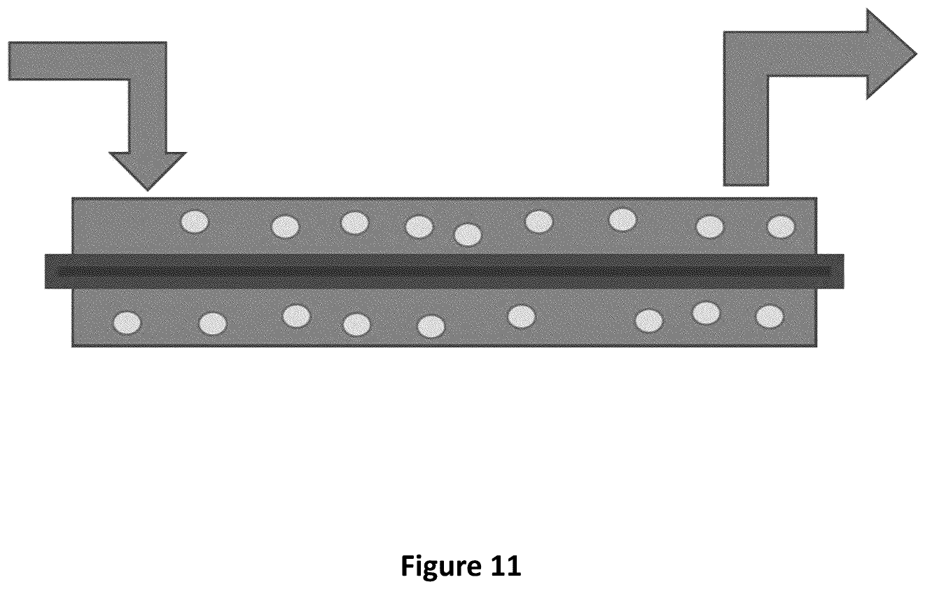

The particles can also be used in a beads+membrane dialysis system (FIG. 10 and FIG. 11) in which adsorbents (beads) are added to the dialyzer (filter) and the dialysate is recirculated (FIG. 10) or not recirculated (FIG. 11).

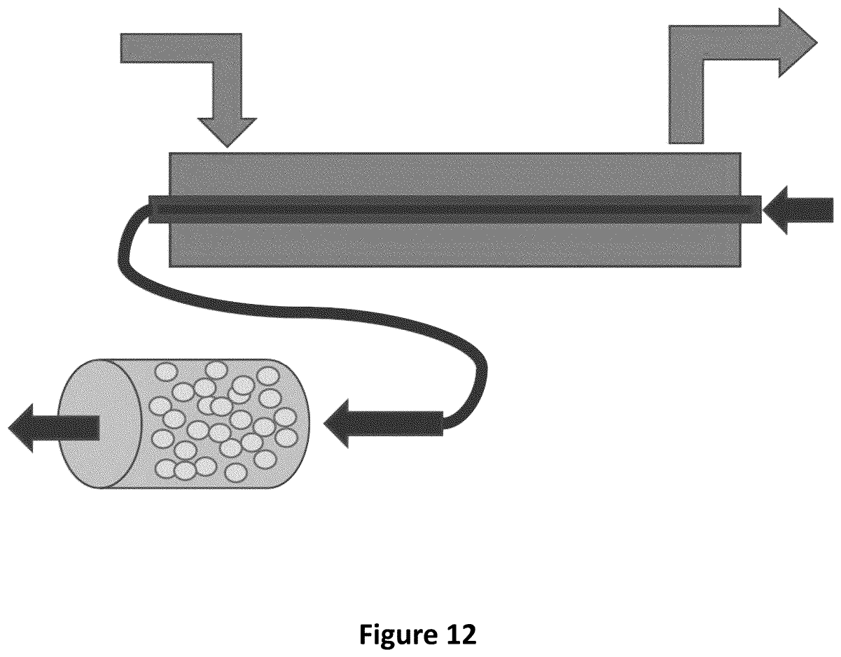

The particles can be used in a cartridge (packed column) system (FIG. 12), in which the adsorbents (beads) are packed in a column, the blood is in direct contact with the absorbents, and the dialysate is not recirculated This approach is known as hemoperfusion.

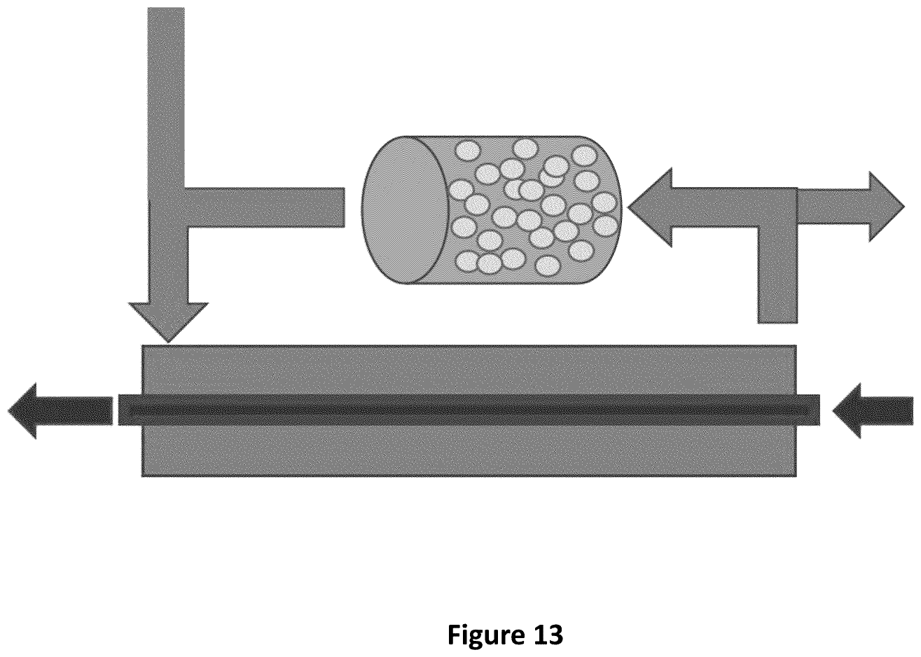

The particles can also be used in a cartridge (packed column) for dialysate purification (FIG. 13), in which adsorbents (beads) are packed in a column for dialysate purification and the dialysate is partially recirculated. An exemplary predicate device is the Recirculating Dialysis (REDY) Sorbent System. In this type of column, the packed column often includes multiple layers for a stepwise purification and can include purification materials such as zirconium oxide, zirconium carbonate, zirconium phosphate, urease, and activated carbon in addition to the particles described herein.

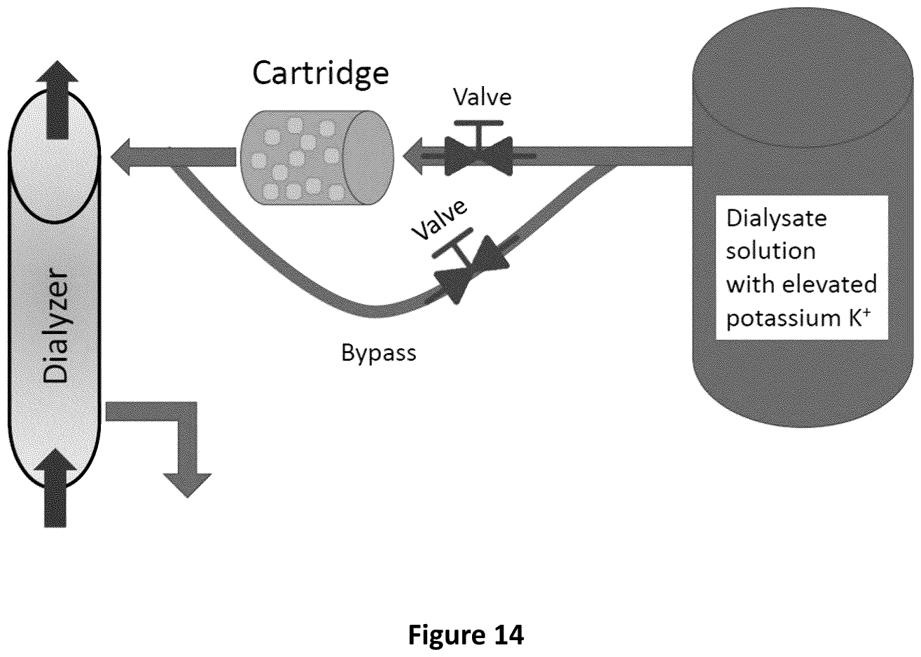

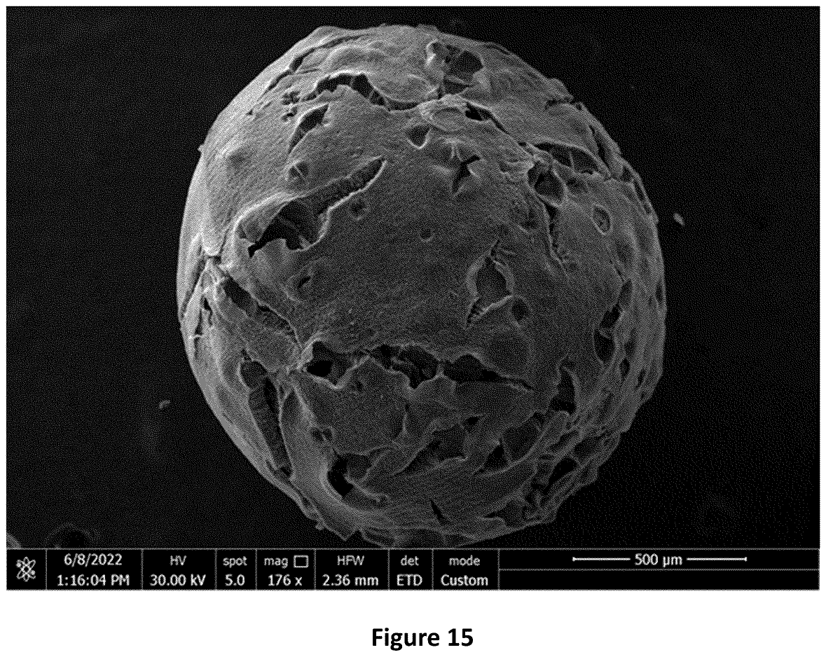

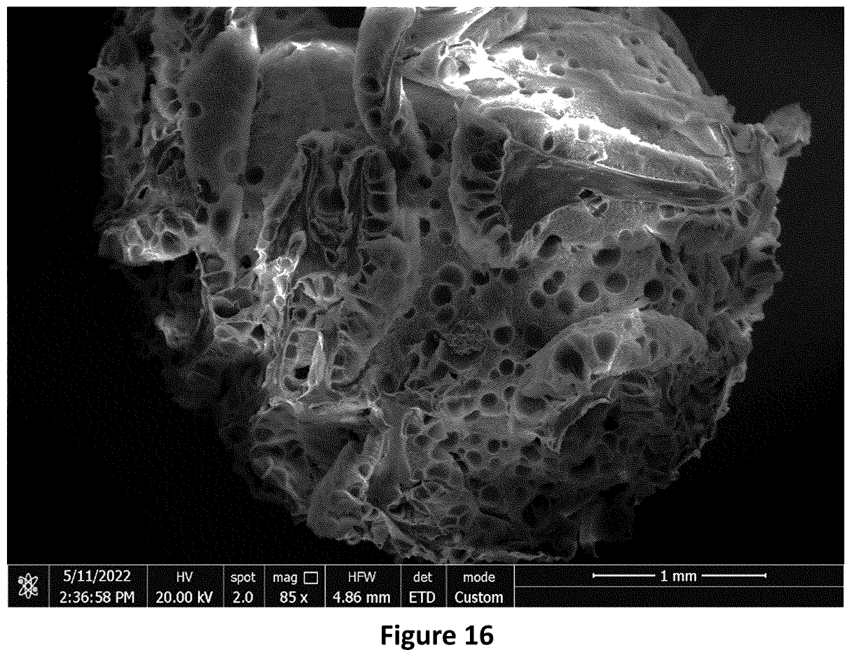

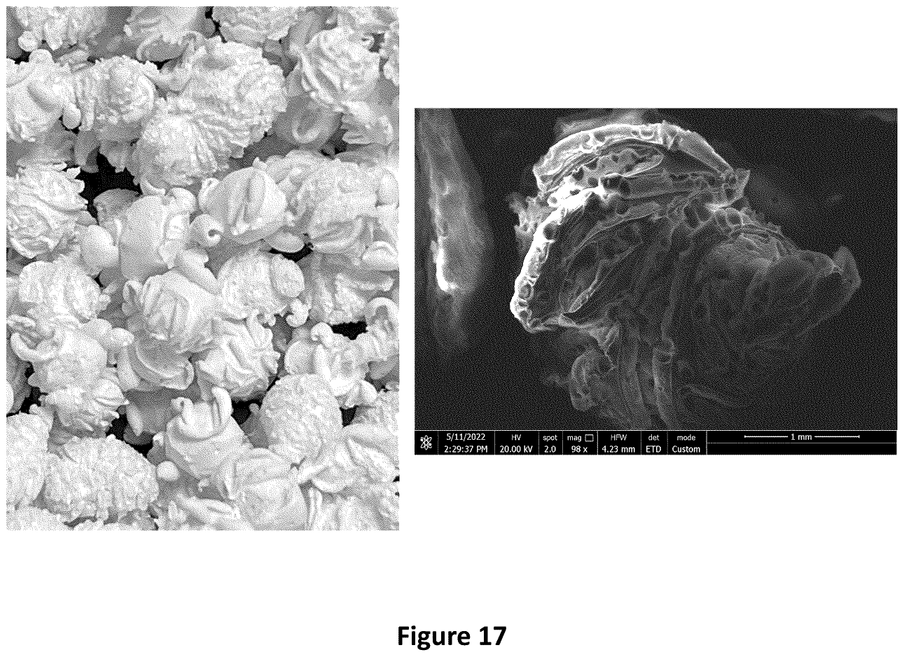

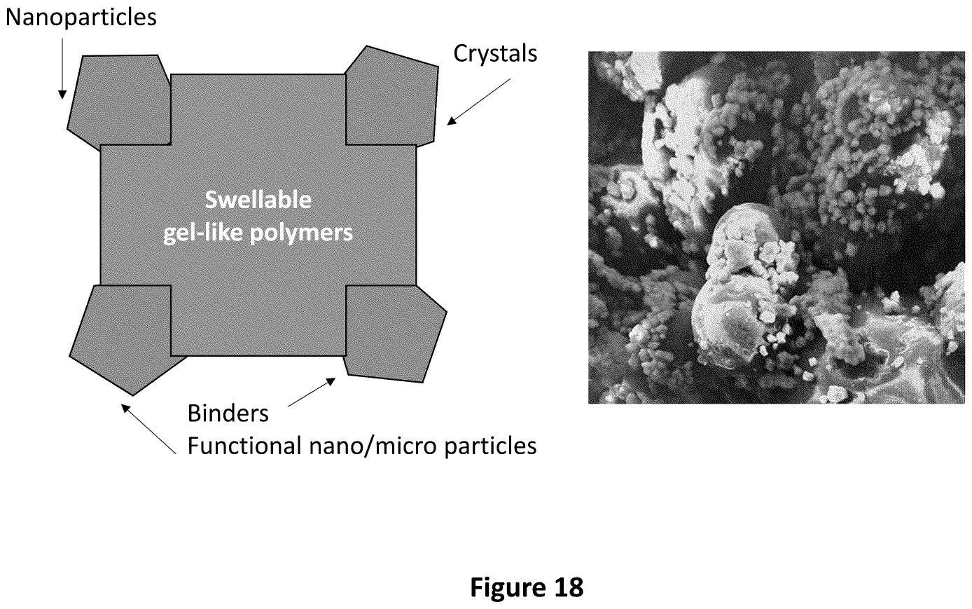

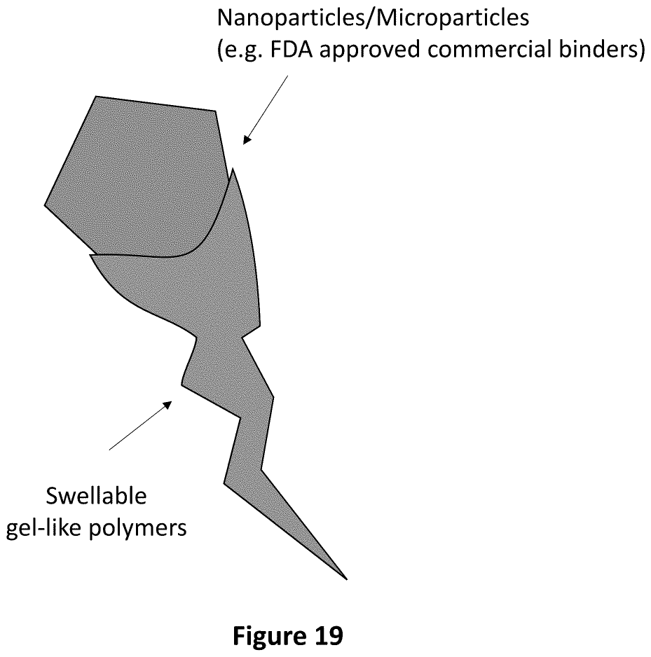

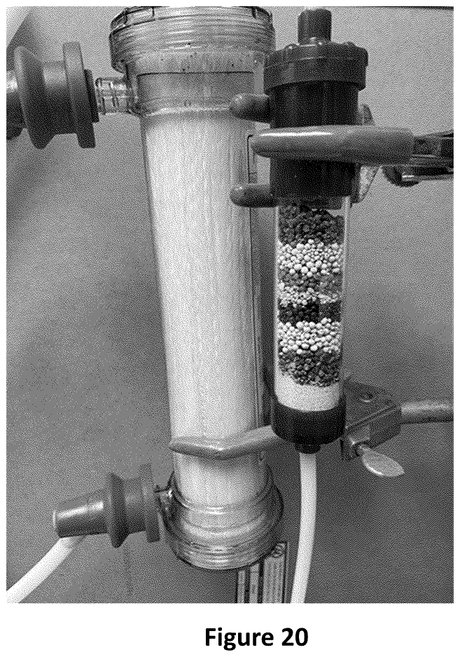

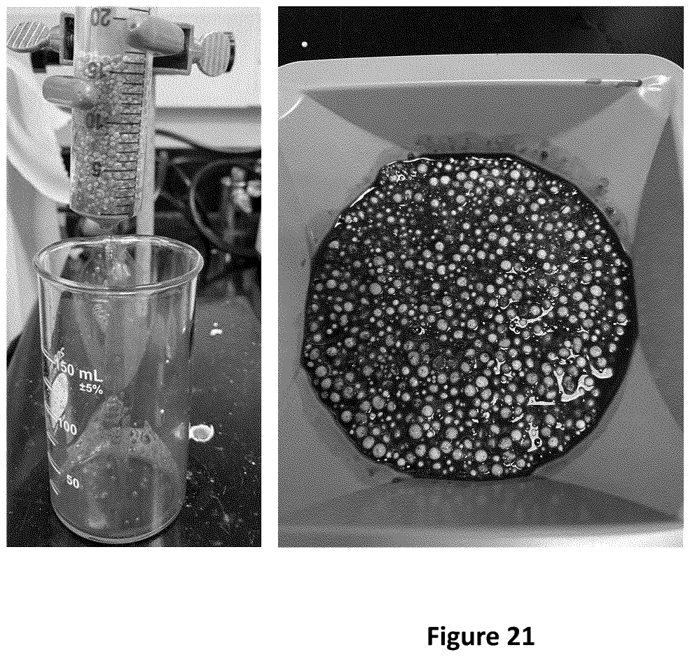

The particles can also be used in a system for stepwise decrease of dialysate potassium (FIG. 14). The macro pores and active sites on the outer surfaces of chitosan-based porous resin can be seen in FIG. 15. The porous fins of a chitosan-based porous resin can be seen in FIGS. 16 and 17. FIG. 18 depicts a design concept for an engineered swellable gel-like drug delivery system containing active binders (adsorbents), catalysts, and slow releasing desorbents. FIG. 19 depicts a design concept for an engineered swellable gel-like drug delivery system. A column can be packed with multiple types of beads for hemoperfusion as shown in FIG. 20. An example of hemoperfusion using biocompatible chitosan-based beads is shown in FIG. 21.

A careful review of literature and extensive discussions with clinicians leads to a defined range of expected potassium concentration for hyperkalemic patients, for example 6.5 to 5.5 mmol/L of potassium. To avoid patients experiencing a high concentration gradient of potassium during dialysis, potassium concentration in dialysate is increased in the method. For example, the potassium level can be increased to a level of 4.5 mmol/L of potassium dialysate solution relative to a more typical 2 to 3 mmol/L; these values can be adjusted by adding KCl. Adding medical-grade KCl (potassium chloride) to dialysate is a common practice. The cartridge depicted in FIG. 14 includes a potassium adsorbent (e.g., a material which selectively absorbs potassium); the whole system comes with 2 tubes and 2 valves. In the beginning of treatment, the cartridge is bypassed and patients are dialyzed using high concentration potassium dialysate, offering low potassium concentration gradient between blood and dialysate. Over the course of treatment, the bypass valve is closed, and the cartridge valve is opened. Using this method, the dialysis process with a high concentration potassium dialysate, and potassium is gradually from the dialysate over the course of treatment. This system provides controlled removal of potassium by offering a low concentration gradient over the course of treatment.

### Example 2: Potassium Removal Using Sodium Zirconium Cyclosilicate as a Binder

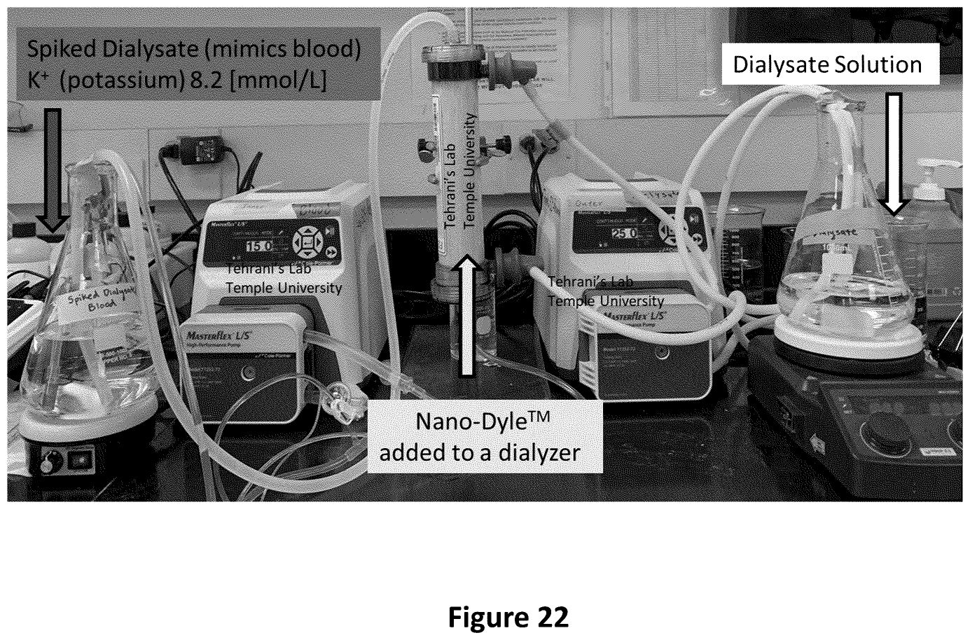

Here, Nano-Dyle™ is gel-like beads made of Polymers (polystyrene) and sodium zirconium cyclosilicate through microwave synthesis. Sodium zirconium cyclosilicate is an FDA-approved potassium binder for oral administration. About 2 grams of beads are inserted into Fresenius OptiFlux 250 dialyzers as adsorbent for potassium management. An experimental setup is shown in FIG. 22.

Here, the objective of this dialysis method is minimizing the potassium concentration gradient between blood and dialysate in a step-wise approach. The potassium level (concentration) in a commercially available dialysate is increased by adding a potassium source to the commercial dialysate. For example, the potassium concentration can be increased to 4 mmol/L. When a patient comes with an elevated potassium level (in this experiment, about 8 mmol/L, which is exceptionally high), the patient is dialyzed with a dialysate with an elevated potassium level (4 mmol/L), and the adsorptive cartridge is bypassed. In a step-wise or on-and-off approach, dialysate flow is passed through a cartridge to gradually decrease the potassium level in dialysate and maintain a gradient between blood and dialysate.

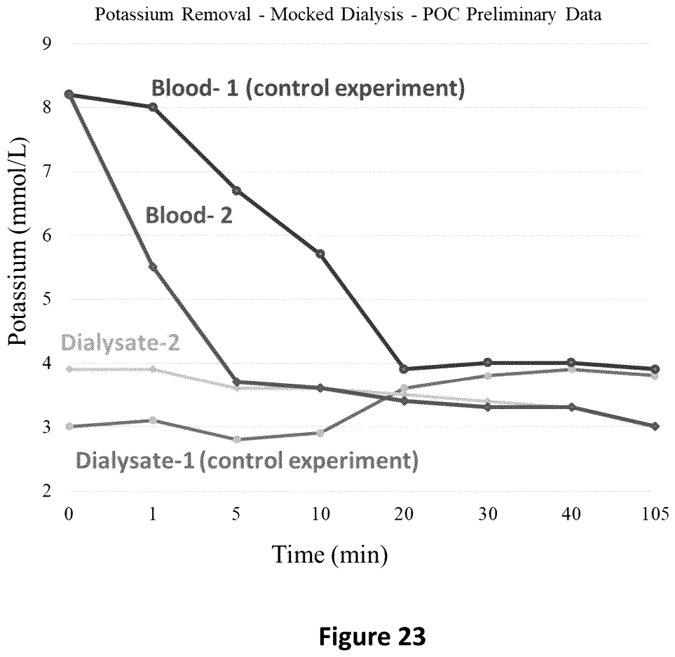

FIG. 23 is a plot of the experimental results. results. The following legends are used in the chart: Blood-1: a dialysate solution is spiked with potassium (K) to mimic blood with an elevated potassium concentration (8.2 mmol/L). This solution was dialyzed using a dialysate solution (3 mmol/L) and dialyzer with no materials added. This is a control experiment. Dialysate-1: dialysate solution was used as a cleansing solution to dialyze Blood-1. (control) An equilibrium of 3.8 mmol/L was reached after 105 minutes. Blood-2: a dialysate solution is spiked with potassium (K) to mimic blood with elevated potassium concentration (8.2 mmol/L). This solution was dialyzed using a dialyzer filled with Nano-Dyle™ and dialysate solution (K 3.9 mmol/L). Dialysate-2: a dialysate solution was used as a cleansing solution to dialyze Blood-2. In this experiment, Nano-Dyle™ was added to the dialyzer.

Significance of this approach for a dialyzed patient: The Patient's blood was dialyzed for potassium removal with a lower concentration gradient. This treatment exerts less cardiac stress.

$$Control \text{:} 8.2 - 3 = 5.2 mmol / L concentration gradient$$

$$Nano - Dyle \text{:} 8.2 - 3.9 = 4.3 mmol / L concentration gradient$$

Also, using Nano-Dyle™, potassium concentration was removed at a much faster rate. Compare blood-1 (control) to blood-2 (Nano-Dyle). A lower concentration of K′ was achieved over 105 mins when compared to control.

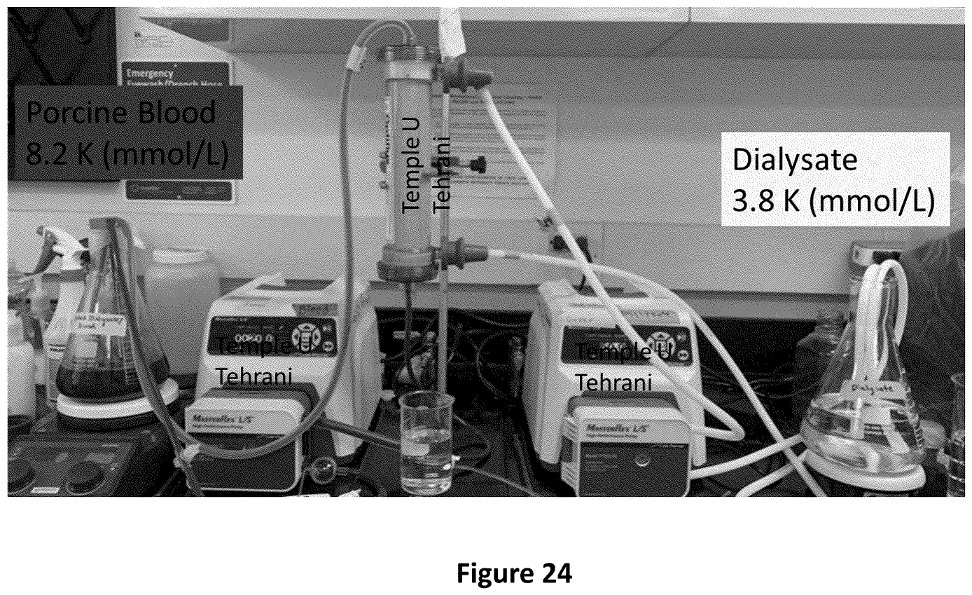

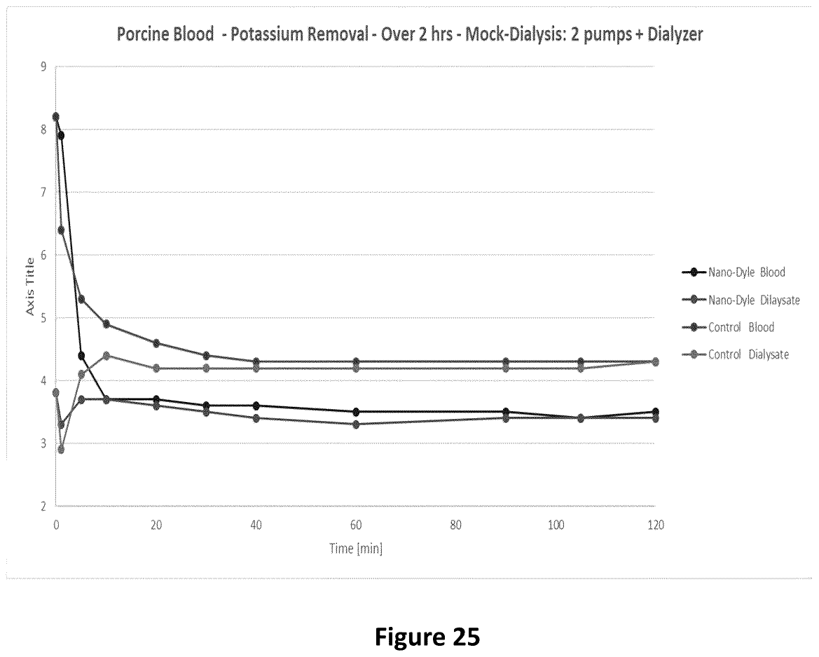

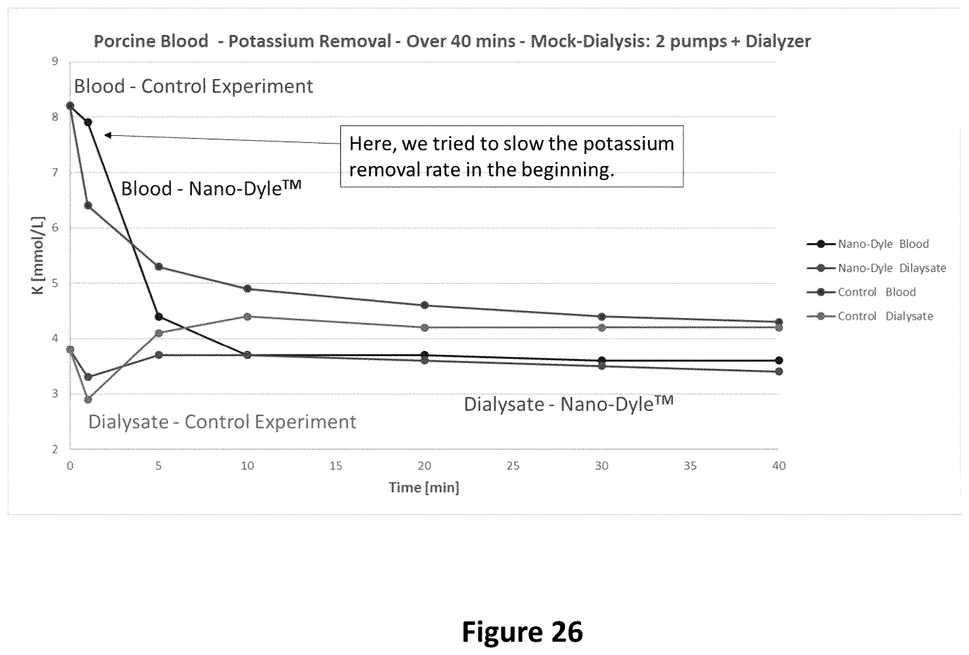

An experimental setup for dialyzing porcine blood with Initial Potassium Conc. 8.2K (mmol/L) is shown in FIG. 24. A plot of the results are shown in FIG. 25. Further analysis of the plot over the first 40 min is shown in FIG. 26.

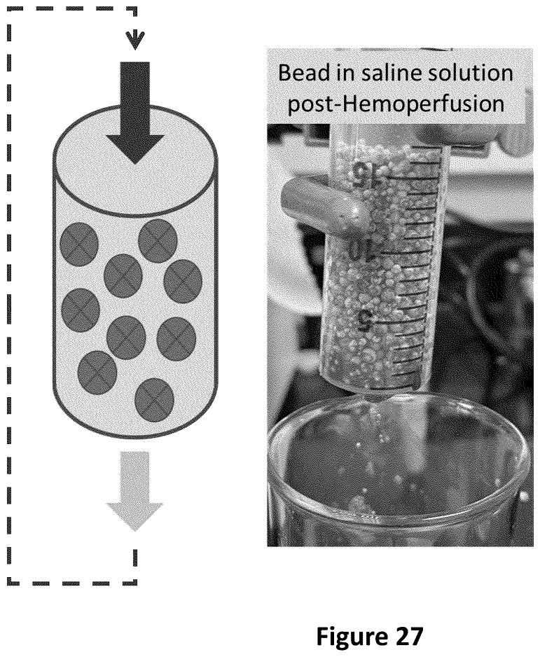

Based on these results, chitosan beads with sodium zirconium cyclosilicate can be used in a hemoperfusion approach for potassium removal. Blood samples with elevated potassium levels are passed through a column of chitosan beads (FIG. 27). Chitosan beads contain sodium zirconium cyclosilicate for selective removal of potassium from blood.

Porcine blood, diluted by 50%, was subjected to hemoperfusion using the bead column. 40 ml of diluted porcine blood was passed through 2 gr of beads. Due to high concentration of potassium in blood, blood was diluted by saline solution Blood was dripped over 2 gr of beads in a column. After 5 full passes, potassium concentration was lowered from 6.9 to 3.5 mmol/L (Table 2). The dashed lines in FIG. 27 show the passage of the blood through the column.

TABLE 2
Hemoperfusion of porcine blood over 5 passes.

| | Diluted Blood | 1^{st} | 2^{nd} | 3^{rd} | 4^{th} | 5^{th} |
|---|---|---|---|---|---|---|
| | (diluted 50%) | Pass | Pass | Pass | Pass | Pass |
| K (mmol/L) | 6.9 | 6.3 | 5.7 | 5.1 | 4.3 | 3.5 |

Porcine blood, diluted by 50%, was subjected to hemoperfusion using the bead column. The initial potassium concentration was 12.6 mmol/L. 100 ml of porcine blood was dripped over the 1 gr of beads in a column. After 14 full passes, potassium concentration was lowered from 12.6 to 7.5 mmol/L (Table 3).

TABLE 3
Hemoperfusion of porcine blood over 14 passes.

| | Diluted Blood | 12^{th} | 14^{th} |
|---|---|---|---|
| | (diluted 50%) | Pass | Pass |
| K (mmol/L) | 12.6 | 7.9 | 7.5 |

FIG. 81 depicts a series of SEM-EDS images of Chitosan-lanthanum composite. FIG. 82 depicts phosphate removal in porcine blood by porous chitosan particles and porous chitosan-Lanthanum particles. FIG. 83 depicts image of syringe used for hemoperfusion experiment and table of results of 50 ml of blood passed through the porous chitosan/La (3.5 g).

### Example 3: Synthesis and Application of Nano and Micro Engineered Porous Beads in Hemodialysis and Hemoperfusion

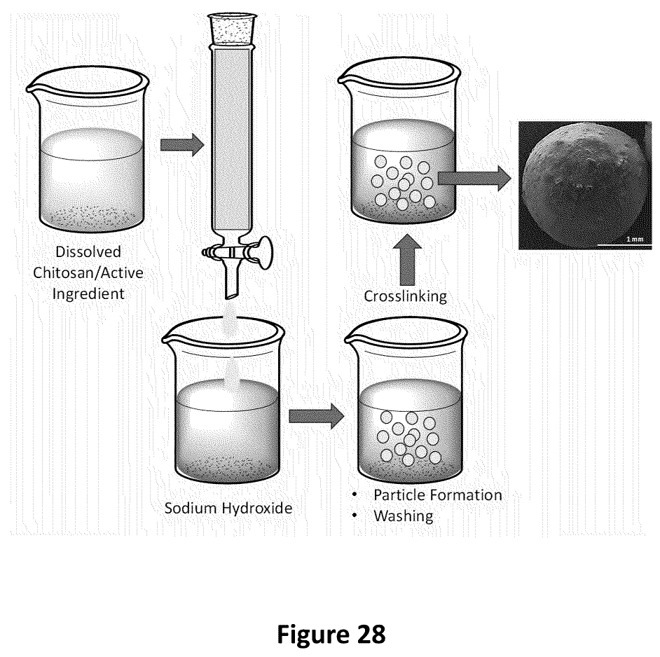

Azodicarboxamide is used to create macro pores, such as in polyethersulfone and chitosan beads using, for example, a droplet method. An exemplary droplet method is shown in FIG. 28. Microwave synthesis of polystyrene/divinylbenzene beads with azodicarboxamide affords gel-like beads.

### Synthesis of Cross-Linked Polystyrene Particles

A solution of styrene (1 ml), divinylbenzene (0.3 ml), heptane (0.5 ml), 2,2-Azobis(2-methylpropionitrile) (AIBN, recrystallized from methanol 99%, 10 mg) and azocarboxamide (300 mg) was prepared and then added to 20 ml of the water phase (a solution of polyvinyl alcohol (10 g/L), sodium chloride (10 g/L), and polyethylene glycol (10 g/L) in water). The prepared solution was added to a 30 ml reaction vial that was sealed with a septa cap and placed in a microwave reactor (Monowave 300, Anton Parr). The dispersion was gradually heated to 40° C. for 10 min, 60° C. for 10 min, 90° C. for 60 min. The reaction dispersion was then quickly cooled down to room temperature with pressurized airflow. The product was collected using the vacuum filter and washed with ethanol and DI water. Microwave synthesis of crosslinked polystyrene/divinylbenzene particles are shown in FIG. 79.

### Synthesis of Cross-Linked Polystyrene-Lokelma Particles

A solution of styrene (1 ml), divinylbenzene (0.3 ml), heptane (0.5 ml), 2,2-Azobis(2-methylpropionitrile) (AIBN, recrystallized from methanol 99%, 10 mg) and Lokelma (300 mg) was prepared and then added to 20 ml of the water phase (a solution of polyvinyl alcohol (10 g/L), sodium chloride (10 g/L), and polyethylene glycol (10 g/L) in water). The prepared solution was added to a 30 ml reaction vial that was sealed with a septa cap and placed in a microwave reactor (Monowave 300, Anton Parr). The dispersion was gradually heated to 40° C. for 10 min, 60° C. for 10 min, 90° C. for 60 min, 90° C. for 60 min. The reaction dispersion was then quickly cooled down to room temperature with pressurized airflow. The product was collected using the vacuum filter and washed with ethanol and DI water. Microwave synthesis of crosslinked polystyrene/divinylbenzene particles are shown in FIG. 80.

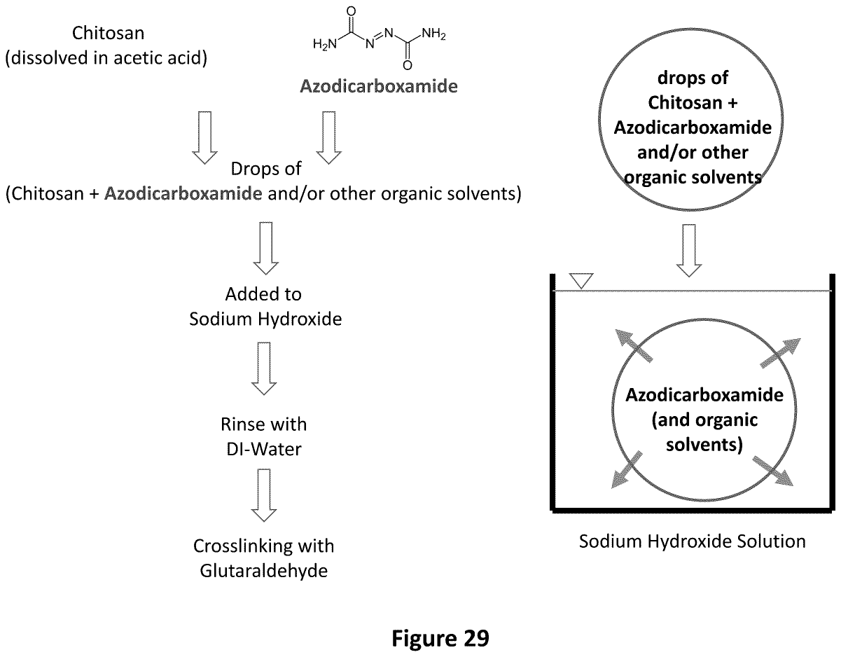

In another exemplary method, Polymers are mixed with volatile and/or hydrophobic or hydrophilic compounds/solvents. Using drop, microfluidic, or spray systems polymers, as drops, are inserted to a solution that has affinity for the added compound/solvent. The added compounds escape the drop and forms pores in the beads. Alternatively, temperature can be used to generate pores, using volatile solvents to escape the beads. Certain volatile solvents are carefully selected so they can evaporate at room temperature and generate pores (FIG. 29).

In another method, polystyrene/divinylbenzene beads can be made using azodicarbonamide to synthesize porous gel-like beads. A pharmaceutical-grade microwave, such as an Anton Paar Monowave 300 microwave, is used to synthesize polystyrene/divinylbenzene gel-like beads (FIG. 18). Gel-like porous spheres can be synthesized with an added FDA-approved potassium binder, LOKELMA (sodium zirconium cyclosilicate), as a functional material on the surface. Also, polymer tails can be added to nano/macro particles/crystalline/amorphous materials.

Azodicarbonamide is used in the food industry as a flour bleaching agent and a dough conditioner. It is known by the E number E927. The principal use of azodicarbonamide is in the production of foamed plastics as a blowing agent.

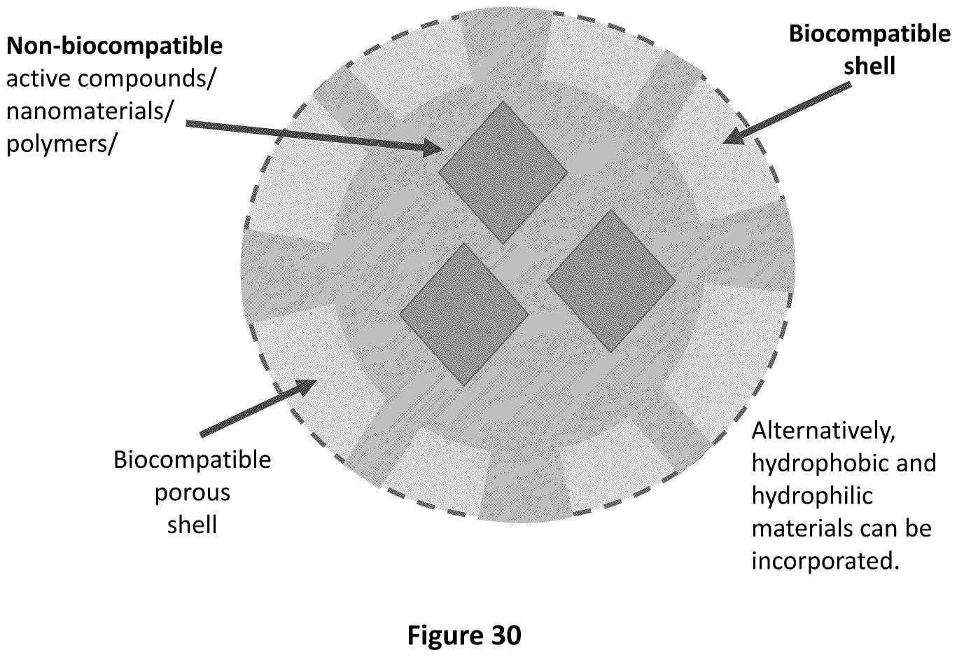

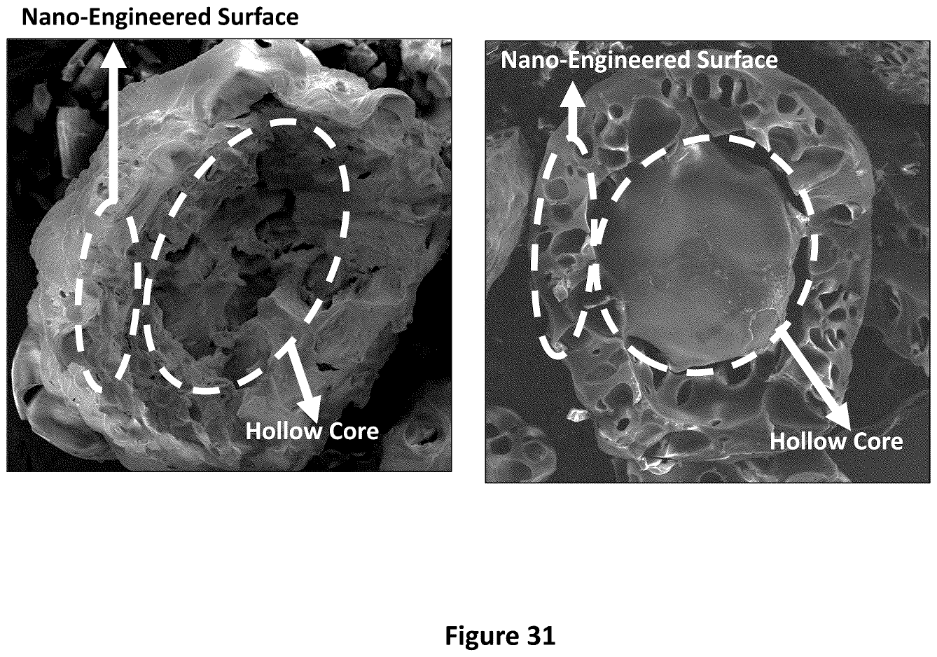

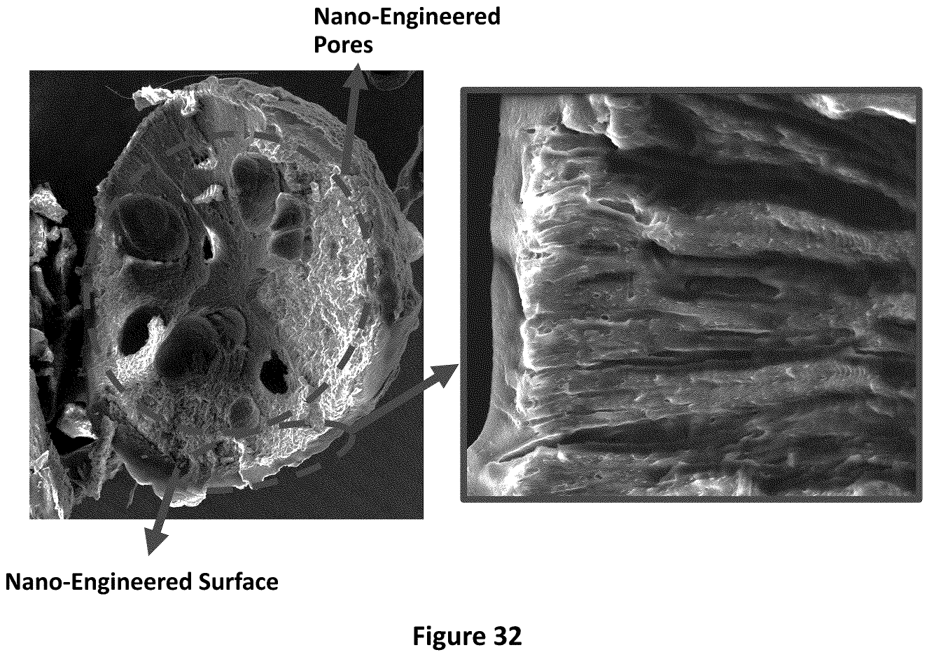

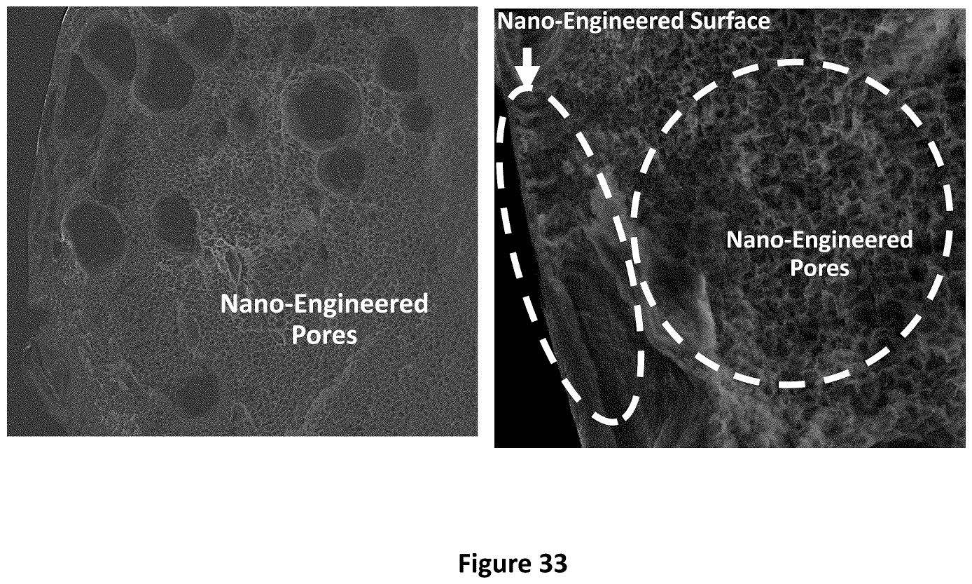

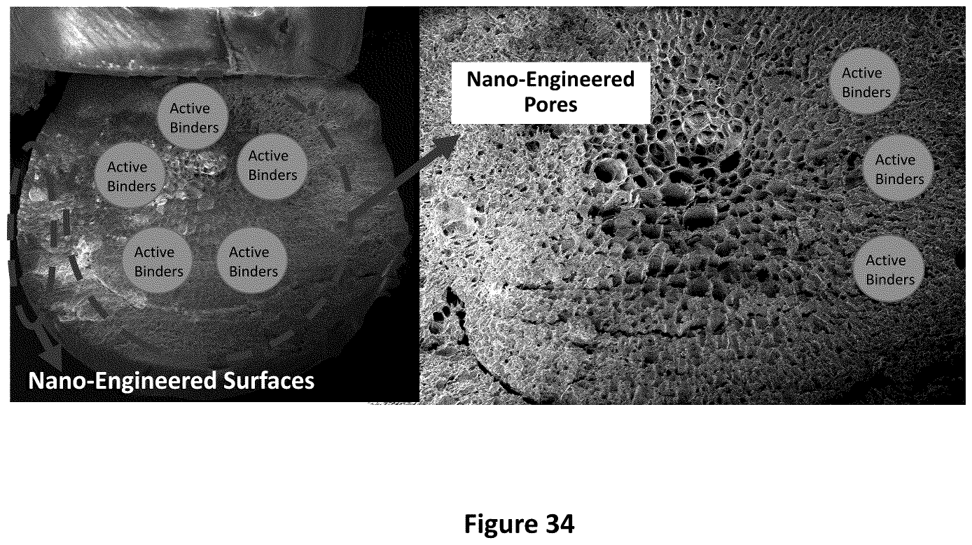

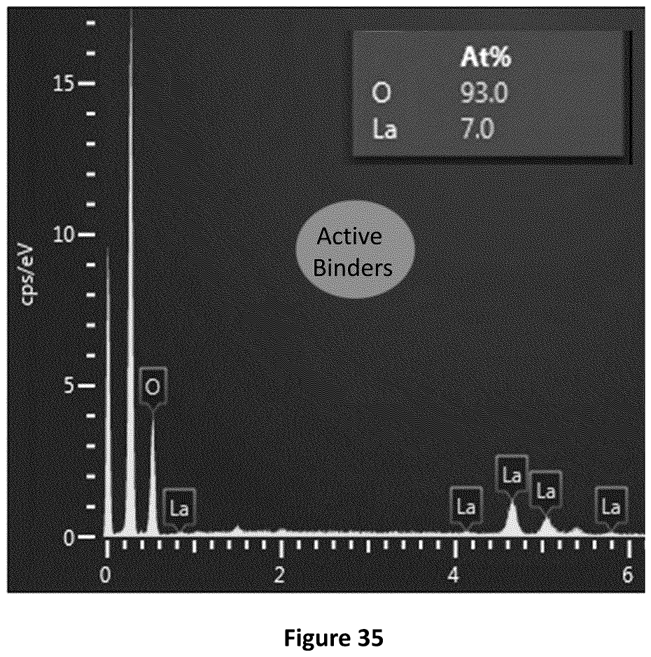

The beads synthesized in this manner can be used to deliver non-biocompatible compounds (FIG. 30). Beads prepared with azodicarbonamide are shown in FIG. 31. Beads prepared with azodicarbonamide and an organic phase are shown in FIG. 32. Beads prepared in an organic phase are shown in FIG. 33. Beads prepared with an organic phase and lanthanum chloride as the active binder for phosphate removal is presented in FIG. 34. FIG. 35 is a plot showing the elemental composition of the resulting particles. A quantification of phosphate removal is presented in Tables 4 and 5.

TABLE 4
Removal of phosphate with lanthanum on chitosan

| Particle | Toxin | Removal | Q (mg/g) |
|---|---|---|---|
| Chitosan + Lanthanum | Phosphate | 31.4% | 2.82 |

TABLE 5
Hyperphosphatemia Management in Hemoperfusion
using Chitosan-Lanthanum Particles

| | Blood Pass | | | | |
|---|---|---|---|---|---|
| | 0 | 1 | 2 | 3 | 4 |
| Phosphorus (mmol/L) | 4.86 | 4.48 | 1.68 | 1.23 | 0.72 |
| pH | 6.766 | | | | 7.086 |

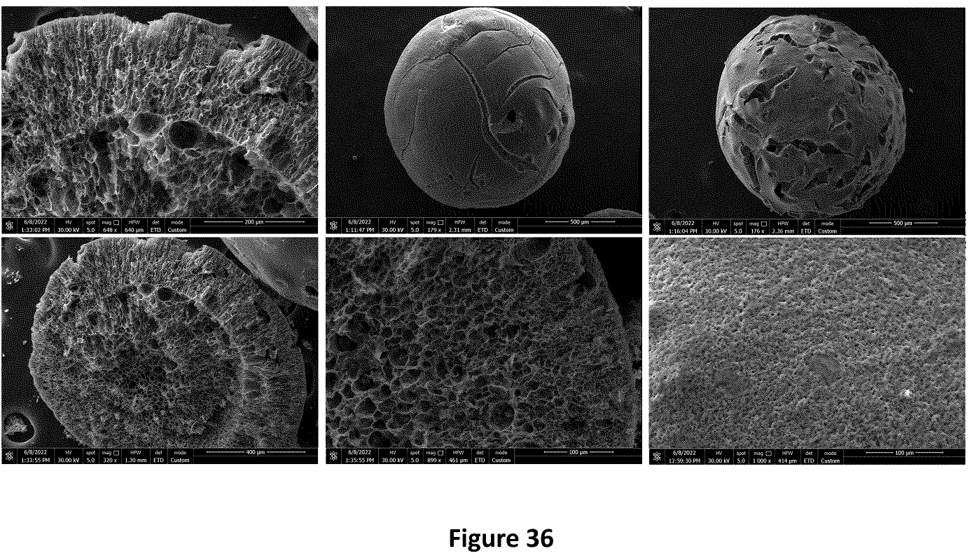

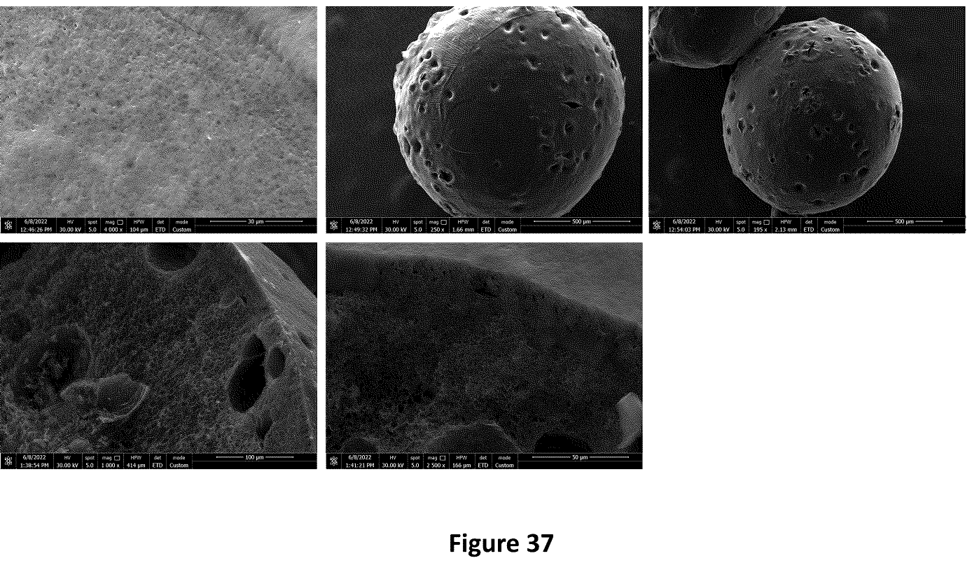

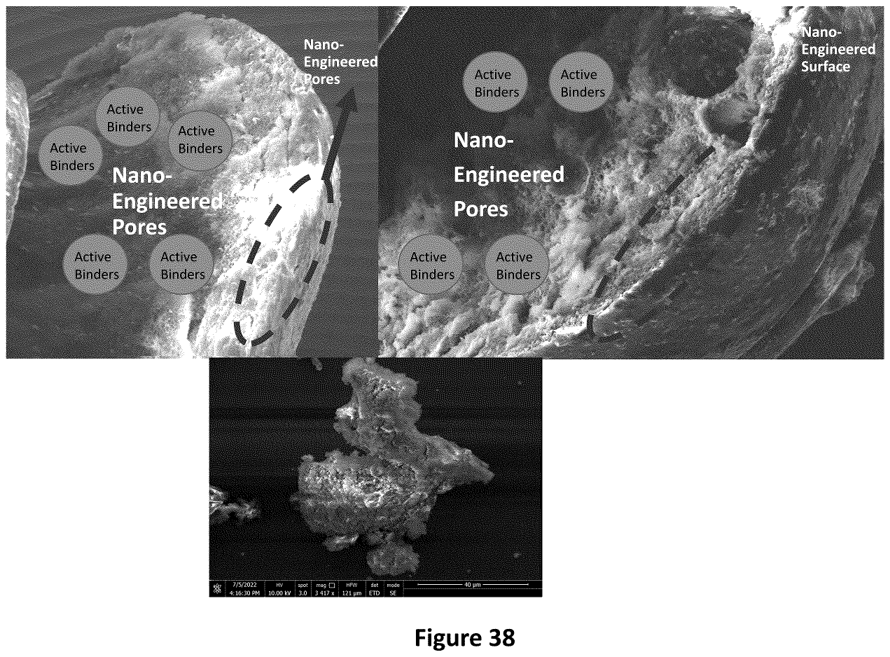

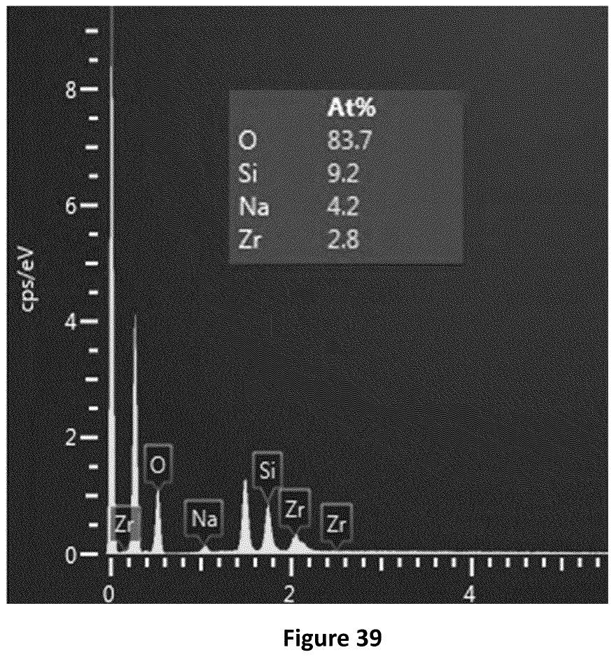

Beads comprising low molecular weight chitosan with Lanthanum, carbonized (pyrolyzed) at 800° C. in nitrogen gas, are shown in FIG. 36. FIG. 37 provides SEM images of more chitosan scaffolds. Beads prepared from the organic phase using sodium zirconium cyclosilicate as the active binder for potassium removal are shown in FIG. 38. FIG. 39 is a plot showing the elemental composition of the resulting particles. A quantification of potassium removal is presented in Tables 6 and 7.

TABLE 7
Removal of potassium with sodium zirconium
cyclosilicate on chitosan

| Particle | Toxin | Removal | Q (mg/g) |
|---|---|---|---|
| Chitosan + sodium | Potassium | 46.9% | 10.68 |
| zirconium cyclosilicate | | | |

TABLE 7
Hyperkalemia Management in Hemoperfusion using Chitosan-Lokelma Particles

| | Blood Pass | | | | | | |
|---|---|---|---|---|---|---|---|
| | (number of passing the same blood sample) | | | | | | |
| | 0 | 1 | 2 | 3 | 4 | 5 | 6 |
| Na (mmol/L) | 145 | 145 | 148 | 149 | 151 | 153 | 155 |
| K (mmol/L | 8.9 | 8.3 | 5.9 | 5.5 | 4.4 | 3.6 | 3 |
| Cl (mmol/L) | 113 | 115 | 115 | 117 | 119 | 120 | 119 |
| iCa (mmol/L) | 1.21 | 1.15 | 0.97 | 0.91 | 0.81 | 0.71 | 0.64 |

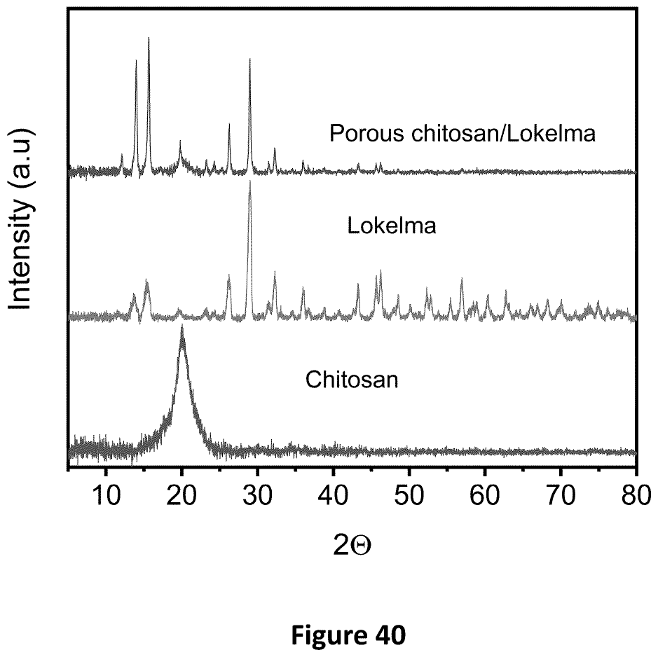

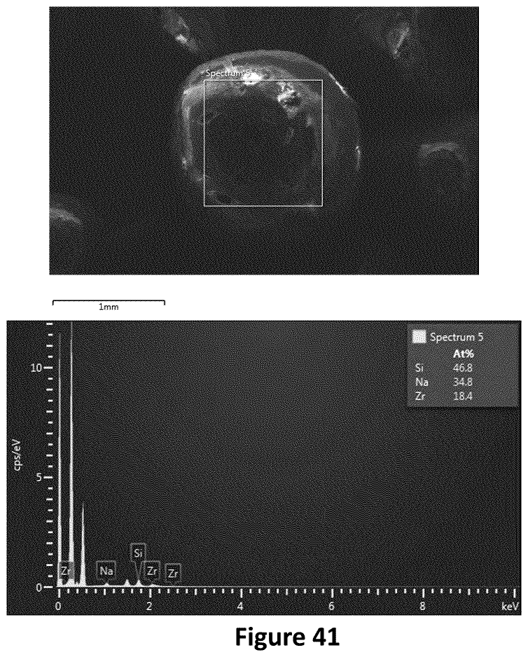

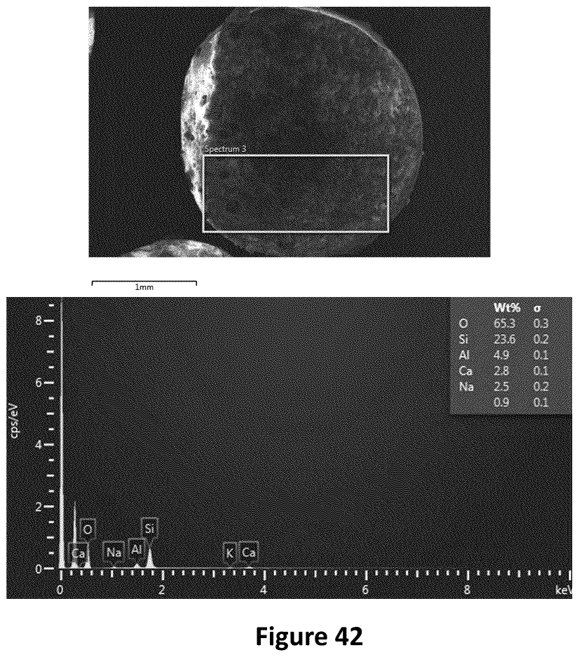

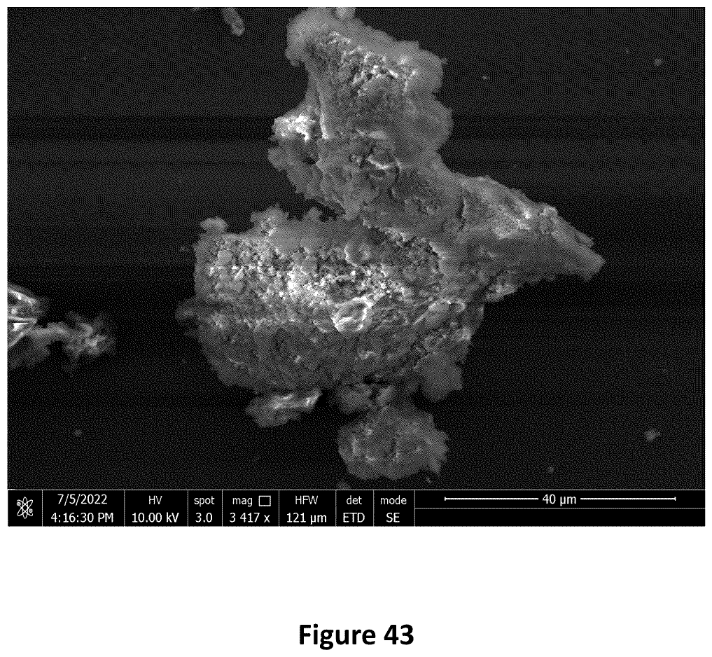

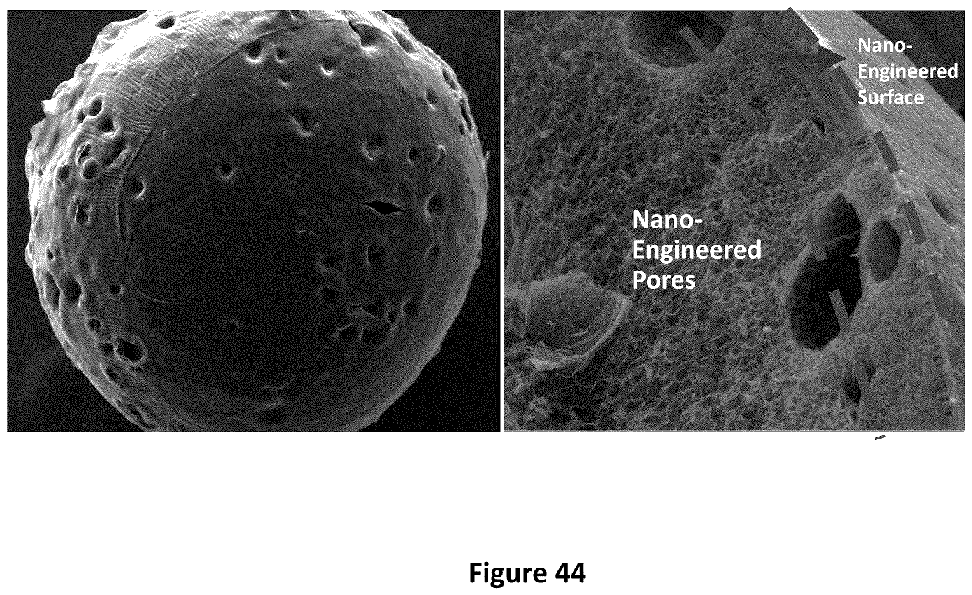

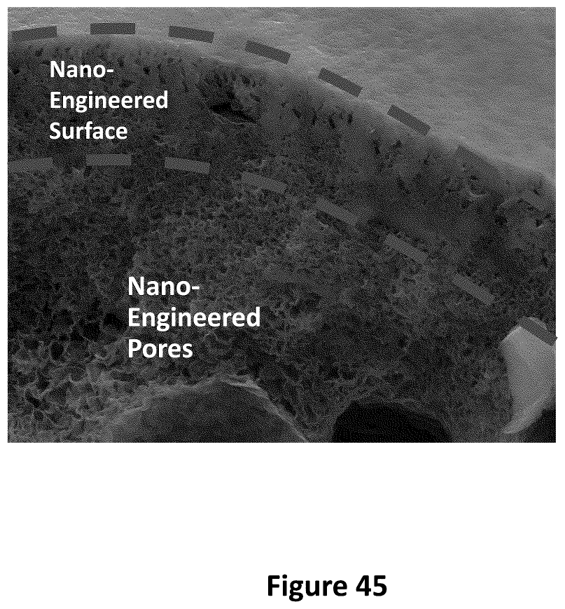

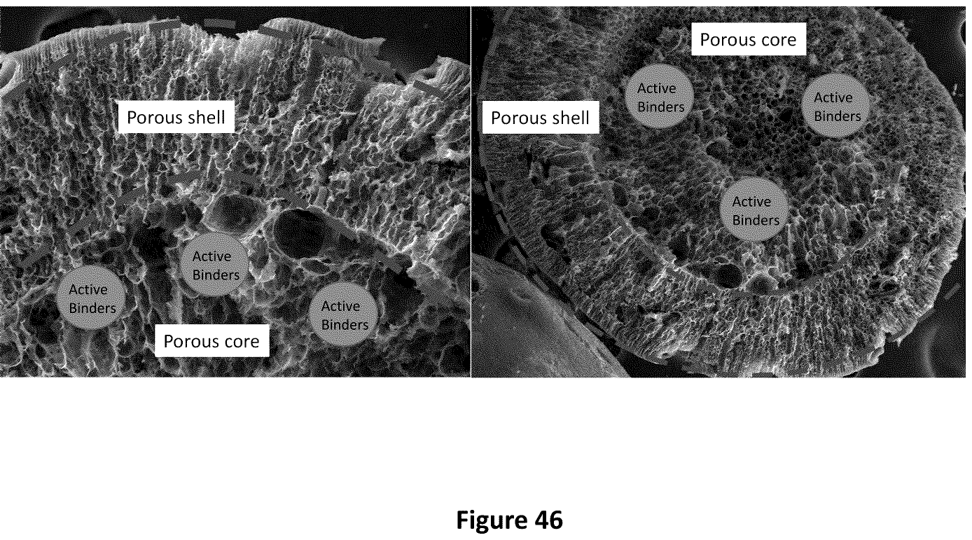

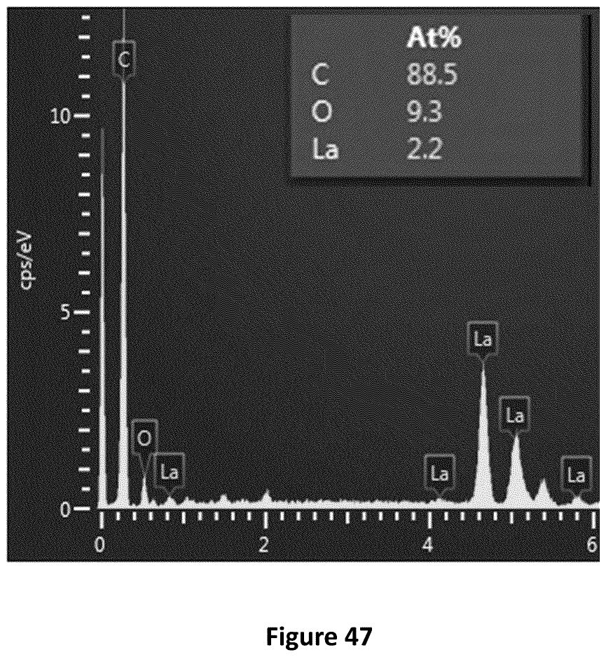

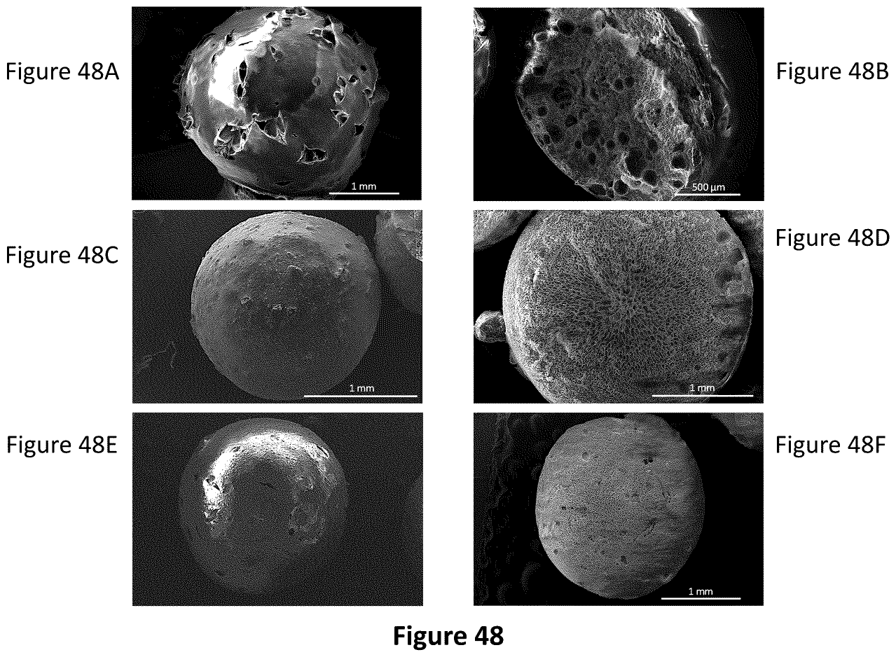

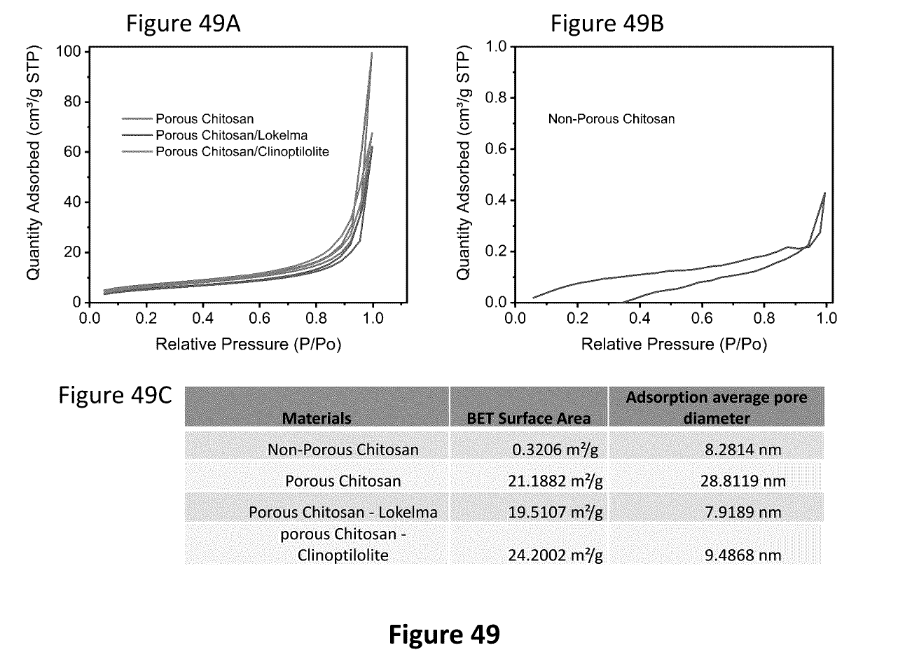

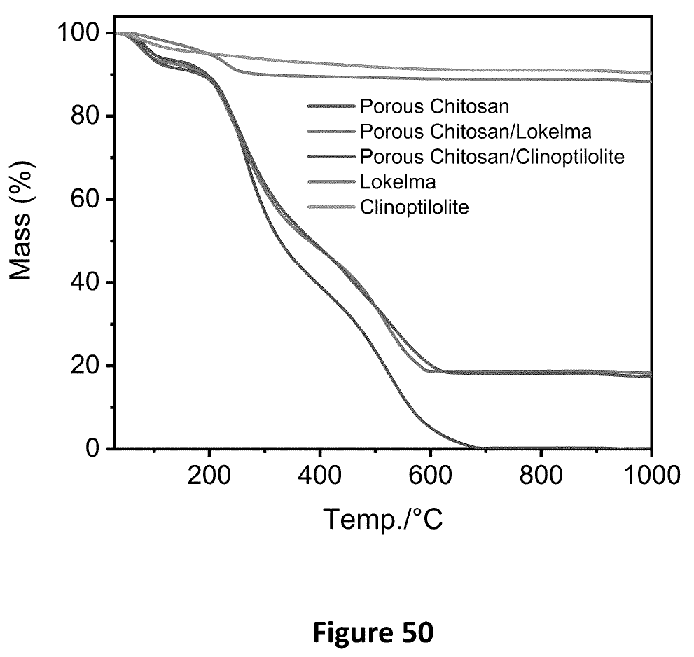

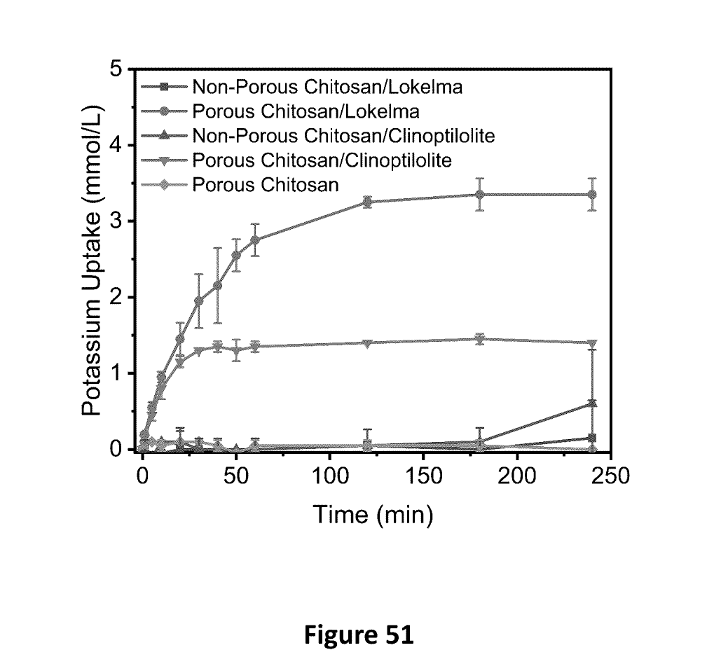

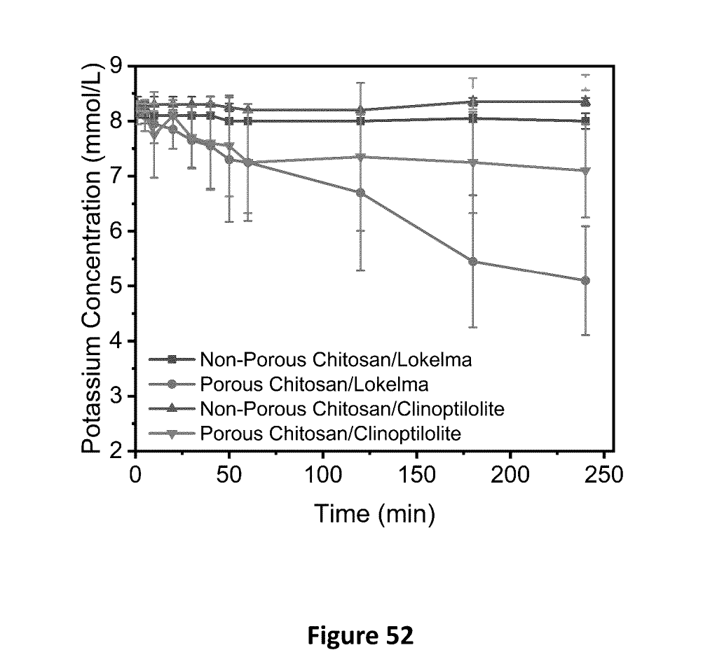

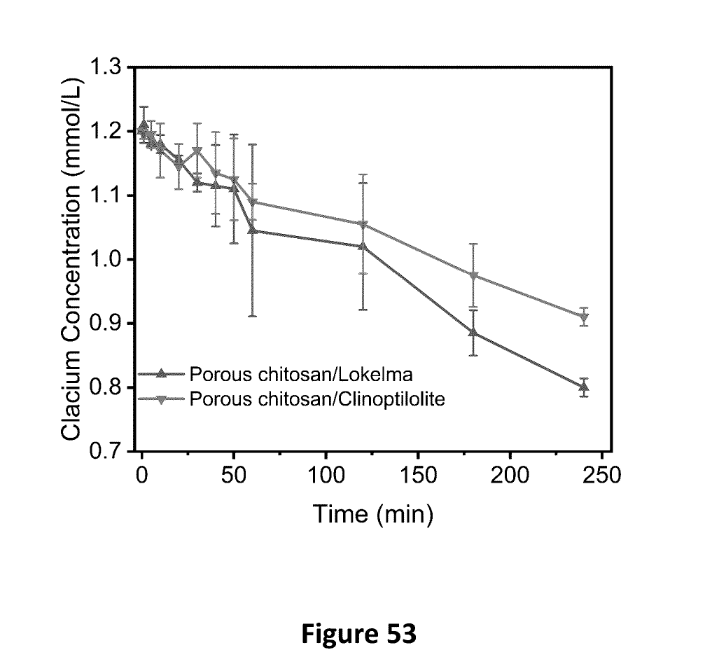

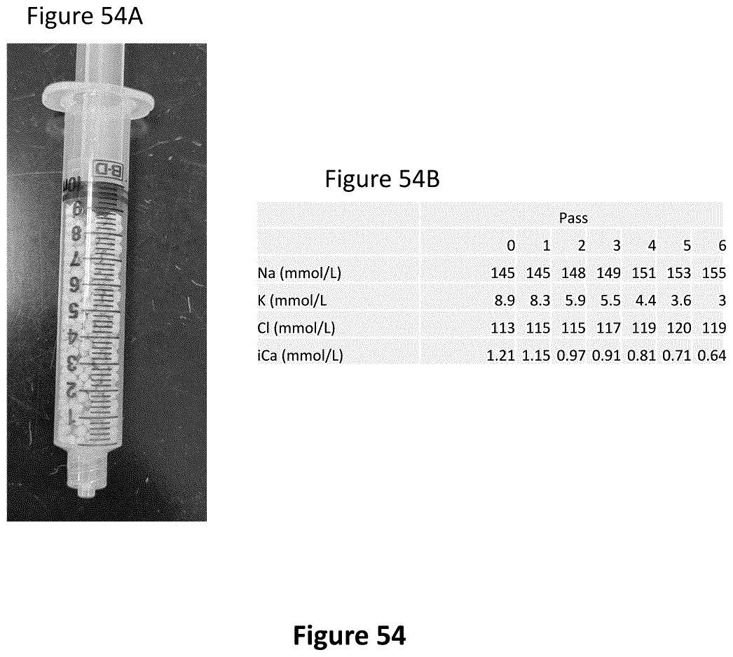

XRD diffractograms of porous chitosan/Lokelma, porous chitosan, and Lokelma are shown in FIG. 40. Another example of porous chitosan/Lokelma is shown in FIG. 41. SEM and EDX of porous chitosan/clinoptilolite are shown in FIG. 42. FIG. 43 is a SEM image of obtained chitosan solution which was sprayed into a NaOH aqueous solution. Beads prepared with an organic phase followed by carbonization are shown in FIG. 44 and FIG. 45. Beads prepared with an organic phase and lanthanum chloride, followed by carbonization, are shown in FIG. 46. FIG. 47 is a plot showing the elemental composition of the resulting particles. FIG. 48 is a series of SEM images of porous chitosan, porous chitosan/Lokelma, and porous chitosan/Clinoptilolite. Brunauer-Emmet-Teller (BET) results from porous chitosan, porous chitosan/Lokelma, and porous chitosan/Clinoptilolite are shown in FIG. 49. TGA experiment results are shown in FIG. 50. Potassium uptake of particles in the dialysate solution is shown in FIG. 51. Potassium removal in porcine blood by chitosan particles is shown in FIG. 52. Calcium removal in porcine blood by porous chitosan/Lokelma and porous chitosan/Clinoptilolite is shown in FIG. 53. FIG. 54 depicts a syringe used for hemoperfusion experiments followed by a table of results from hemoperfusion experiments.

Alternative synthesis method using Lanthanum: 2 g chitosan and 1 g LaCl3 were added to 80 ml (2% acetic acid) and stirred at 70° C. to dissolve them. The obtained chitosan solution was added dropwise into a NaOH aqueous solution (1M). The beads were aged overnight, and then rinsed with DI-water to obtain a neutral pH. Then the beads were added to glutaraldehyde solution (5%) to cross-link the microsphere particle overnight. The cross-linked microsphere particles were washed with DI-water and were dried.

Synthesis of chitosan scaffolds: 1 g chitosan was added to 50 ml (2% acetic acid) and stirred at 70° C. to dissolve chitosan. The obtained chitosan solution was added dropwise into a NaOH aqueous solution (1M). The beads were aged overnight and then rinsed with DI-water to obtain a neutral pH. Then the beads were added to glutaraldehyde solution (5%) to cross-link the microsphere particle overnight. The cross-linked microsphere particles were washed with DI-water and were dried.

Synthesis of Cellulose-Chitosan Scaffolds: 1 g chitosan and 1 g cellulose were added to 80 ml (2% acetic acid) and stirred at 70° C. to dissolve chitosan. The obtained chitosan solution was added dropwise into a NaOH aqueous solution (IM). The beads were aged overnight, and then rinsed with DI-water to obtain a neutral pH. Then the beads were added to glutaraldehyde solution (5%) to cross-link the microsphere particle overnight. The cross-linked microsphere particles were washed with DI-water and were dried.

### Example 4: Novel Synthesis Methods for Fine-Tuning Chitosan Beads: A Balance Between Porosity, Removal Capacity, and Mechanical Strength

Biopolymeric beads and membranes offer environmentally friendly and effective alternatives to conventional fluid purification treatment methods and materials. Biopolymers (such as cellulose, chitin, and their derivatives) have been studied in various fields, such as agriculture, biomedicine, cosmetics, food, and water treatment. They are known for their abundance, biocompatibility, biodegradability, and non-toxicity. The application of chitin is limited because it is insoluble in most solvents, while chitosan is soluble in diluted acidic solutions. Chitosan is extensively used as a scaffold or hydrogel for various applications, from water treatment to tissue engineering. Chitosan is a cationic natural polysaccharide and can be obtained by the deacetylation of chitin, the most abundant natural biopolymer after cellulose (Nasrollahzadeh, M., et al., 2021, Carbohydr. Polym., 251; Olivera, S., et al., 2016, Carbohydr. Polym., 153, 600-618; Dohemdou, M., et al., 2021, J. Biol. Macromol., 192, 771-819). The amino and hydroxyl functional groups in the chitosan structure have motivated scientists to synthesize polymeric adsorbents and composites for water treatment to remove ions, metals, organic compounds, and recently PFAS (Ateia, M., et al., 2019, Environ. Sci. Technol. Lett., 6 (12), 688-695; Liu, W., et al., 2022, Chemosphere, 309, 136733; Zhang, Q., et al., 2011, Bioresour. Technol., 102 (3), 2265-2271; Elanchezhiyan, S., et al., 2021, Carbohydr. Polym., 267, 118165). Additionally, it can be used as polymeric adsorbent beads or scaffolds to embed inorganic adsorptive materials (Gryzbek, P., et al., 2022, Int. J. Mol. Sci., 23 (17); Muixika, A., et al., 2017, Int. J. Bio. Macromol., 105 (Pt 2), 1358-1368; Mi, F. L., et al., 1999, Biomaterials, 20(17), 1603-1612; Wang, M., et al., 2017, Scientific Reports, 7 (1), 1-11; Misra, S. K., & Pathak, K., 2022, Chitosan in Biomed. App., 37-73).

Numerous studies have explored diverse synthesis methods and their resulting physical attributes of chitosan (Nasrollahzadeh, M. et al., 2021, Carbohydr, Polym., 25 251:116986; Olivera, S. et al., 2016, Carbohydr, Polym., 153:600-618; Gryzbek, P. et al., 2022, Int. J. Mol. Sci., 23 (17); Yaashikaa, P. R., et al., 2022, Environ. Res., 212, 113114; Keshvardoostchokami, M., et al., 2021, Carbohydr, Polym., 2021, 273, 118625). Particularly in medicine, where cellulose and chitosan are of considerable interest, there has been an effort to adjust the physiochemical characteristics of biopolymeric scaffolds (Dohendou, M., et al., 2021, Int. J. Biol. Macromol., 192, 771-819; Misra, S. and Pthak, K., 2022, Chitosan in Biomedical App., 37-73; Nussinovitch, A., 2010, Polym. Macro- and Micro-Gel Beads: Fund. and App., 1-303; Shu, X. Z., and Zhu, K. J., 2002, Int. J. Pharm., 233 (1-2); Li, B., et al., 2020, J. Mater. Chem. B., 8 (35), 7941-7946). Optimizing the design, synthesis, and application of these biopolymers for large-scale production and application poses a multifaceted challenge, encompassing engineering physical, chemical, and operation attributes such as particle size and porosity, cost, scalability, quality control, and a range of critical material properties like swelling and mechanical strength.

Particle Size and Porosity: As chitosan beads are used in a wide range of applications, controlling the size and porosity of the particles plays a crucial role in the physical and chemical properties of the particles and their application (Mi, F., et al, 1999, 20 (17), 1603-1612; Xu, J. H., et al., 2012, Adv. Healthc. Mater., 1 (1), 106-111; Zhou, H. et al., 2013, 229, 82-89). The porosity of the chitosan can play a vital role in the efficiency of polymer for fluid purification. Introducing porous morphology to the particles provides active sites and surface area for removing pollutants and toxins Gryzbek, P. et al., 2022, Int. J. Mol. Sci., 23 (17); Rorrer, G., et al., 1993, Ind. Eng. Chem. Res., 32 (9), 2170-2178; Huang, L., et al., 2018, Carbohydr. Polym., 202, 611-620; Yang, Y., et al., 2020, SN. Appl. Sci., 2 (3), 1-10; Park, C., et al., 2016, J. Haz. Mater., 309, 133-150). Particle size and particle size distribution are essential considerations when producing polymeric beads for large-scale industrial applications. The examination of commercially available polymeric beads has revealed that achieving absolute uniformity in particle size is nearly unattainable. However, adhering to manufacturing guidelines regarding particle size distribution can help maintain quality and performance.

Swelling and Mechanical Strength: Swelling and mechanical strength are crucial properties of polymeric beads when considering their application in full-scale water treatment. Swelling, the polymeric bead's ability to absorb water and physically expand its size, directly influences its capacity to capture and remove contaminants from water. Thus, a higher swelling ratio indicates more available and accessible active sites and better removal potential, improving treatment performance. However, excessive swelling can lead to the disintegration of the beads, reducing their lifetime and effectiveness (Saber-Samandari, S., et al., 2015, JOM, 9 (1), 19-25; Wong, S., et al., 2021, Front. Nutr., 8, 752207). On the other hand, mechanical strength measures the bead's resistance to physical and mechanical deformation and breakage. For example, during water treatment, the beads are subjected to various mechanical stresses, pressure changes, and the weight of the adsorption column. High mechanical strength ensures the beads maintain integrity and functionality under field operational conditions. Insufficient mechanical strength can lead to bead breakage, resulting in a loss of treatment capacity and potential release of captured contaminants into the water (Shu, X., Z., and Zhu, K., J., 2002, Int. J. Pharm., 233 (1-2), 217-225; Aryaei, A., et al., 2012, J. Mech. Behav. Mater., 5 (1), 82-89; Chatterjee, S., et al., 2009, Carbon N. Y., (12), 2933-2936.) Both swelling and mechanical strength are critical considerations in designing and producing chitosan beads for full-scale water treatment. A balance between these properties is needed to ensure the beads' durability, effectiveness, and cost-efficiency in real-world applications.

Safety, Biocompatibility, and Biodegradability: Safety is also critical; the beads' gaseous or liquid leachates during the priming phase should be non-toxic and safe to handle to ensure the safety of operators, fluid quality, the environment, or patient's health. Ease of separation after treatment is another crucial factor; beads that can be easily separated from the fluid streamline the treatment process and reduce costs. The efficiency of the bead production process, in terms of yield, can also significantly impact the feasibility and viability of using polymeric beads for large-scale production. Lastly, biodegradability is another key parameter; ideal beads should degrade over time to prevent long-term pollution and facilitate easier disposal. Such parameters are crucial in assessing the suitability and efficiency of polymeric beads for water treatment (Keshvardoostchokami, M., et al., 2021, Carbohydr. Polym., 273, 118625; Nussinovitch, A., 2010, Fund. and App., 1-103; Aryaei, A., et al., 2012, J. Mech. Behav. Biodmed. Mater., 5 (1), 82-89; Chatterjee, S., et al., 2009, Carbon N. Y., 47 (12), 2933-2936; Rasel Das, Polymeric Materials for Clean Water, 2019; Cheremisinoff, P., 1997, Hand. Of Eng. Polym. Mater.; Williams, P. A., Hand. Of Ind. Wat. Sol. Polym., 2007, 331; Forster, A., M., 2023, Materials Testing Standards for Additive Manufacturing of Polymer Materials: State of the Art and Standards Applicability; Polymeric Adsorbent Resins for Industrial Applications, 2023, Purolite; Spalding, M., A., et al., 2016, Hand. Of Ind. Polyeth. Tech., 1-1352).

The initial step in engineering chitosan beads revolves around adjusting key parameters such as particle size, porosity, pore volume, and their distributions (Cheremisinoff, P., 1997, Handbook of Eng. Poly. Mat.; Williams, P. A., 2007, Handbook of Ind. Wat. Sol Poly.; Spaldin, M. A., and Chaterjee, A. M., 2016. Handbook of Ind. Polyeht. Tech.). These modifications aim to optimize the performance of these materials for specific target molecular weights. Each targeted toxin or toxin class requires a particular set of physical properties for effective and selective removal (Olivera, S., et al., 2016, Carbohydr. Polym., 153, 600-618; Gryzbek, P., et al., 2022, Int. J. Mol. Sci., 23 (17); Cheremisinoff, P., 1997, Hand. Of Eng. Polym. Mater.; Williams, P. A., Hand. Of Ind. Wat. Sol. Polym., 2007, 331; Forster, A., M., 2023, Materials Testing Standards for Additive Manufacturing of Polymer Materials: State of the Art and Standards Applicability; Spalding, M., A., et al., 2016, Hand. Of Ind. Polyeth. Tech., 1-1352).

Material Physicochemical Characterization: The use of various analytical techniques, such as ATR-FTIR (Attenuated Total Reflectance-Fourier Transform Infrared Spectroscopy), TGA (Thermogravimetric Analysis), and XRD (X-ray Diffraction) can provide a comprehensive characterization of chitosan beads. ATR-FTIR spectroscopies are practical spectroscopy tools for characterizing chitosan beads. FTIR provides molecular-level insights, such as identifying functional groups, and monitors structural changes. It identifies various characteristic bands corresponding to O—H, N—H, C—H stretching, and acetyl group presence in chitosan. ATR, an FTIR-based technique, enhances surface sensitivity and allows for analyzing chitosan beads' surfaces without extensive sample preparation. It helps detect subtle changes on the bead surfaces. These techniques collectively offer a robust understanding of chitosan beads' structural and chemical characteristics (Durak, T., and Depciuch, J., 2020, Environ. Exp. Bot., 169, 103915; Pawlak, A., and Mucha, M., 2003, Thermochim. Acta., 396 (1-2), 153-166).

Thermogravimetric analysis (TGA) is a key method for characterizing chitosan, providing insights into its thermal stability and degradation behaviors. TGA tracks the weight loss of chitosan as a function of increasing temperature, identifying distinct stages of degradation. The initial weight loss at lower temperatures helps determine the amount of absorbed and bound water. Subsequent weight loss at higher temperatures can be attributed to the decomposition of chitosan, where specific gases like water, ammonia, carbon monoxide, carbon dioxide, and acetic acid are released. The temperatures at which these losses occur can reveal chitosan's stability and chemical composition, allowing researchers to understand its properties and potential applications (Pawlak, A., and Mucha, M., 2003, Thermochim. Acta., 396 (1-2), 153-166; Corazzari, I., et al., 92015, Polym. Degrad. Stab., 112, 1-9; Brady, J., et al., 2009, Developing Solid Oral Dos. Forms: Pharm. Theory. and Prac., 287-217; Prestov, A., and Bratskaya, S., 2016, Mol., 21 (3), 330).

X-ray diffraction (XRD) is a potent tool for analyzing the structural properties of chitosan polymeric beads. XRD can provide critical insights into the crystalline nature of the beads by identifying characteristic diffraction peaks associated with chitosan. These peaks' intensity, position, and width can be used to determine crystallinity, phase composition, and possible structural changes due to processing or modifications. For example, peak broadening may suggest decreased crystallinity or the presence of a polymorphic structure. Therefore, XRD is a valuable method for comprehensive structural analysis of chitosan beads (Eddya, M., et al., 2020, Heliyon, 6 (2); Clark, G., and Smith, A., 1936, J. of Phys. Chem., 40 (7), 863-879).

Imaging: SEM (scanning electron microscope) imaging was done by FEI Quanta 450 FEG SEM equipped with energy-dispersive X-ray spectroscopy (EDS). The instrument was used at 5 kV and the samples were mounted on carbon silica. Fluorescent imaging was done using an Olympus Fluorescent microscope. The porous and non-porous chitosan microspheres were individually placed in the microscope with 1 mg/L fluorescein isothiocyanate isomer I (90%) dye. The microscope was at 10× magnification and different z-stack images were captured.

Adsorption Studies: Nitrogen isotherms were performed at 77 K using an ASAP2460 instrument. The specific surface area was calculated using Brunauer, Emmett, and Teller (BET), Langmuir, and t-plot external. The total pore volumes were calculated from the amount of nitrogen adsorbed at P/P0, for each sample. The total pore volumes were calculated from the amount of nitrogen adsorbed at P/P0, 0.9947, 0.9956, 0.9944, and 0.9947 for the control, PCP-M, PCP-T, and PCP-A, respectively. X-ray powder diffraction patterns of tuned chitosan microspheres and non-porous chitosan microspheres were obtained by a Bruker D8 X-ray diffractometer with Cu Kα radiation (λ=0.15406 nm). Samples were scanned from 5-80° (2θ value), 0.02° step, and 0.3 s per step.

Methylene blue is favored as a model adsorbate in adsorption studies due to its high visibility, standardization across studies, accurate measurability via spectrophotometric analysis, environmental stability, and safe handling characteristics. Methylene blue removal was tested using a UV-Vis spectrophotometer. The UV-Vis spectrum was obtained using DR 5000 UV-Vis spectrophotometer for porous and non-porous chitosan microspheres at 664 nm (number of replicates, n=2). A prior study was conducted at pH 2, 4, and 6 to determine the optimum pH for efficient adsorption, and a pH of 6 was selected for future adsorption studies. A calibration curve was plotted using 0.1, 0.2, 0.5, 1.0, and 2.0 mg/L concentrations of methylene blue (MB) solution.

Mechanical Strength: The synthesized non-porous and porous microspheres were tested for their mechanical properties using Caldaro position sensors (Caldaro sadae, Japan) and Deben Microtest tensile stage. Deben Microtest software (V6.1.51) was used to process the data, and deformation resistance was calculated from the nominal stress and strain curve using Hertz model. The ramping velocity during this work was 0.2 mm/min. Furthermore, the microspheres were soaked in water for 1 hour to determine the effect of dampness on the mechanical strength.

Porogen leaching, one of the most established polymer processing techniques, fabricates porous products. The method involves dispersing a template within a solution composed of polymers or monomers, solidifying the structure, followed by template removal to achieve a porous morphology. This technique applies to a diverse range of polymers and hydrogels. The process of dissolving “sacrificial structures” is a recurring feature in numerous fabrication strategies (Humacher, D. W., et al., 2023, Tissue Eng. Third. Ed., 355-385).

A new new synthesis technique to impart elasticity to a rigid chitosan polymeric bead by forming hollow-sphere microstructures is presented. Chitosan microspheres were prepared by dissolving chitosan polymer in diluted acetic acid forming a hydrogel, where an increase in the pH of the polymer solution deprotonated the chitosan, reducing its solubility and led to the formation of microsphere particle by van der Waals forces, H bonding, and hydrophobic interactions (Mahaninia, M. H., and Wilson, L. D., 2016, J. Appl. Polym. Sci., 133 (5); Yang, Y., et al., 2020, SN Appl. Sci., 2 (3), 1-10). The conjugated chitosan polymers were made porous by introducing surfactant and porogen additives by three techniques. In the one method (or in the first method), the solvent extraction method was employed based on the oil-in-water (O/W) emulsification technique.

In another method, a polysorbate surfactant (polysorbate 20) was added to the polymer solution and removed from the polymer microsphere particle through the synthesis process. Polysorbate 20 (Tween 20), a surfactant, has received approval from the U.S. Food and Drug Administration (FDA) for its use as an additive in both food and pharmaceutical products. The last method is based on adding a substance, azocarboxamide (ADA), to the chitosan solution, and then the decomposition of the substance through the synthesis process. ADA is an FDA-approved food additive, used for whitening and conditioning dough. The synthesis of these porosity-tuned microspheres was achieved without any additional synthesis steps.

Synthesis Method: Two grams of low molecular weight chitosan polymer were dissolved in 80 ml glacial acetic acid solution (2%) at 70° C. The obtained chitosan solution was added dropwise into a NaOH aqueous solution (1M). The beads were aged overnight and then rinsed with DI water to obtain a neutral pH. Then the beads were added to glutaraldehyde solution (5%) to cross-link the microsphere particle overnight. The cross-linked microsphere particles were washed with DI water and dried at room temperature. The porous microsphere particles were synthesized through the same procedure but by adding three different additives to the chitosan solution. The first method is based on the addition of the organic phase/surfactant to the polymer solution; 10 ml of 2-methylpentane (as organic phase) and 24 ml of tween 20 (as surfactant) were mixed and then added to the polymer solution, the sample made from this method is named PCP-M. The second method is based on adding a porogen agent (4 g of azocarboxamide) to the chitosan solution. Beads made by using azocarboxamide are named PCP-A. In the last method, 35 ml of tween 20 (a porogen agent) was added to the polymer solution, the PCP-T sample. In summary, PCP stands for porous chitosan particles, M for 2-methylpentane, A for azocarboxamide, and T for tween 20. Low molecular weight chitosan (75-85% deacetylation, and a molecular weight range: 50,000-190,000 Da), glutaraldehyde, azocarboxamide, fluorescein isothiocyanate isomer I (90%), and polysorbate 20 (Tween 20) were purchased from the Sigma-Aldrich company.

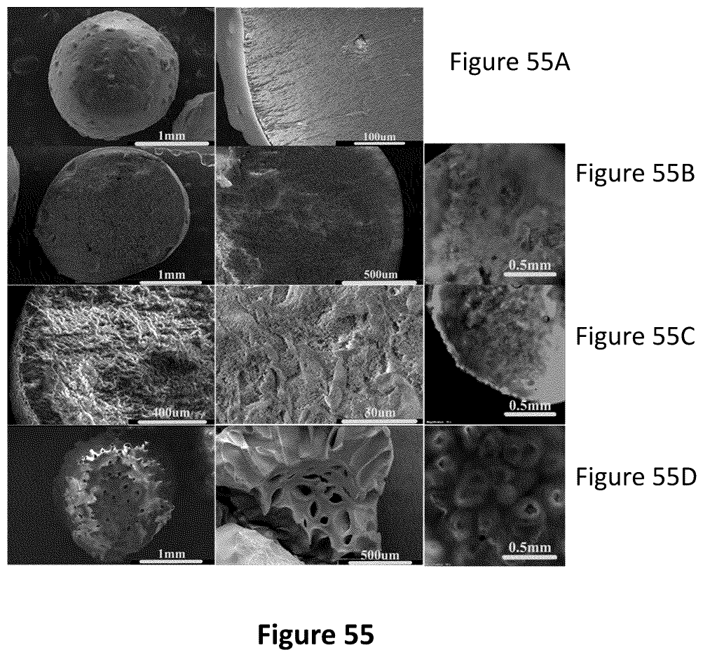

Imaging: SEM and Fluorescent imaging of non-porous microspheres and synthesized PCP-M, PCP-A, and PCP-T are shown in FIG. 55. SEM images of the non-porous microsphere proved the absence of pores, and the FM images did not show any fluorescence due to the lack of pores. PCP-M, PCP-A, and PCP-T microspheres showed porosity in both SEM and PM images. Many factors, including the amount and type of porogen, monomer composition, crosslinking agent, and polymerization conditions, can affect the pore structure. Pore size of PCP-A was the largest, followed by PCP-M. PCP-T had the lowest pore size resulting in high density, infinitesimal porous network. The z-stack of PCP-M and PCP-A microspheres showed highly branched pores and large tunnel-like pores, respectively. A similar structure was observed in the corresponding SEM images, indicating different effects of individual porogen on the structure of microspheres.

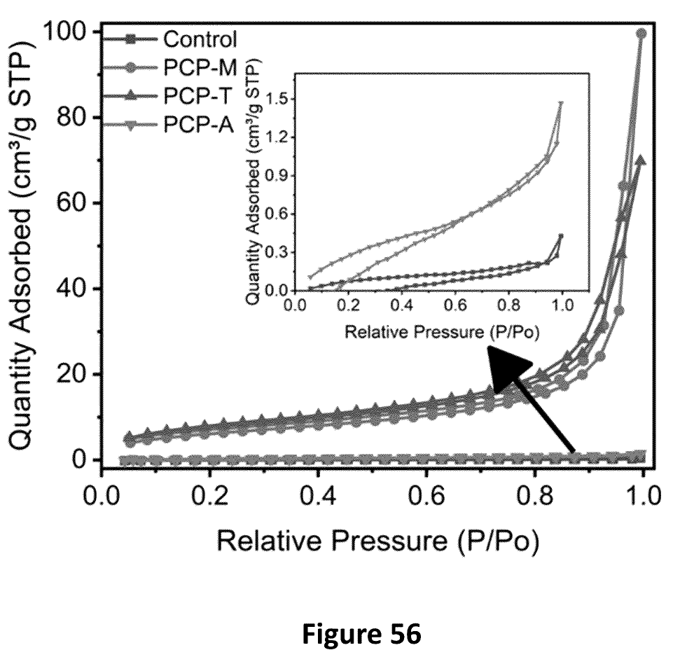

Brunauer, Emmett, and Teller Porosity and Surface Area Analysis: A nitrogen adsorption-desorption experiment was performed to evaluate the particles' surface area, pore size, and pore diameter as shown FIG. 56. International Union of Pure and Applied Chemistry (IUPAC) classified nitrogen adsorption-desorption isotherms; the samples show the type IV behavior with H3 hysteresis loops. Type IV isotherm appears when there is an interaction between gas molecules and adsorbent mesopore surface, leading to capillary condensation. Type IV isotherm corresponds to micro and mesopores in the substance.

The Brunauer-Emmet-Teller (BET), Langmuir, and t-Plot external surface area of samples arise after introducing porosity to the particles (Table 8). The surface area of the PCP-T and PCP-M are significantly higher than the control. Although the surface area of PCP-A is more elevated than the control, it is notably lower than PCP-M and PCP-T. The microporous structure is more dominant in the PCP-A sample, confirmed by SEM results. The results suggest that PCP-T shows a higher surface area and smaller pores than PCP-M.

TABLE 8
Surface area, pore size, and pore diameter of chitosan particles. Total pore
volume and pore diameters were calculated from the N₂ adsorption isotherms.

| | | | | | | BJH | D-H |
|---|---|---|---|---|---|---|---|
| | | | t-Plot | | Adsorption | Adsorption | Adsorption |
| | BET | Langmuir | external | Total pore | average | average | average |
| | Surface | Surface | surface | volume of | pore | pore | pore |
| | Area | Area | area | pores | diameter | diameter | diameter |
| Samples | (m²g) | (m²/g) | (m²/g) | (cm³/g) | (nm) | (nm) | (nm) |
| Control | 0.32 | 1.24 | 0.47 | 6.64*10−4 | 8.28 | 13.06 | 6.92 |
| PCP-M | 21.19 | 111.53 | 23.90 | 0.15 | 28.81 | 30.79 | 9.47 |
| PCP-T | 25.76 | 126.71 | 27.59 | 0.11 | 16.77 | 19.66 | 16.07 |
| PCP-A | 1.23 | 6.84 | 1.72 | 2.272*10−3 | 7.37 | 10.03 | 6.46 |

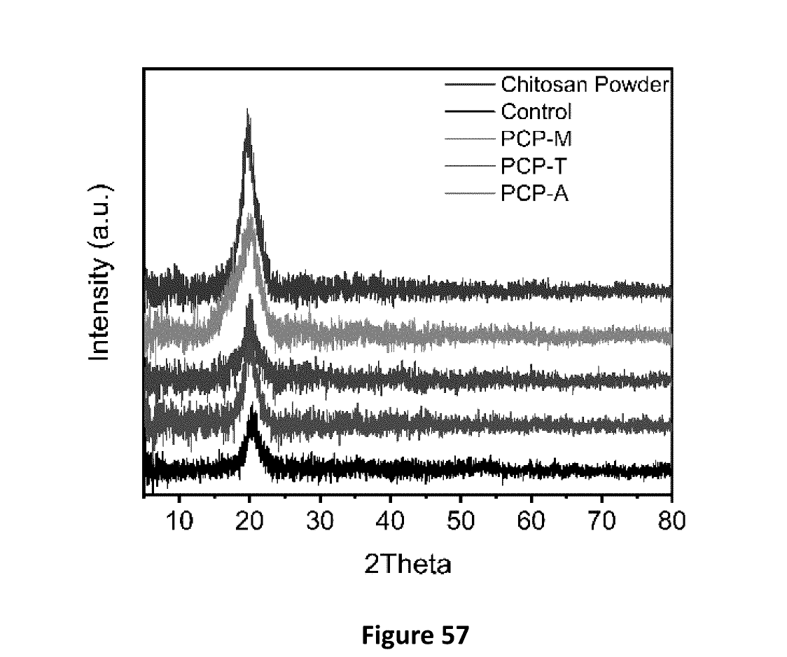

XRD Analysis: FIG. 57 shows XRD patterns of the powdered, synthesized non-porous, and porous chitosan microspheres. From the pattern, the two characteristic peaks at 10° and 20° of the extracted chitosan with a crystalline structure. Powdered pristine chitosan had an intense peak at 20° with a sharp peak corresponding to the chitosan phase. Similar peaks of the chitosan phase were observed in synthesized chitosan. The peak broadening observed in the porous chitosan microsphere at 20° indicates the polymorphic structure and deviation from perfect crystallinity. Among the synthesized porous microspheres, PCP-M showed the sharpest peak at 20°.

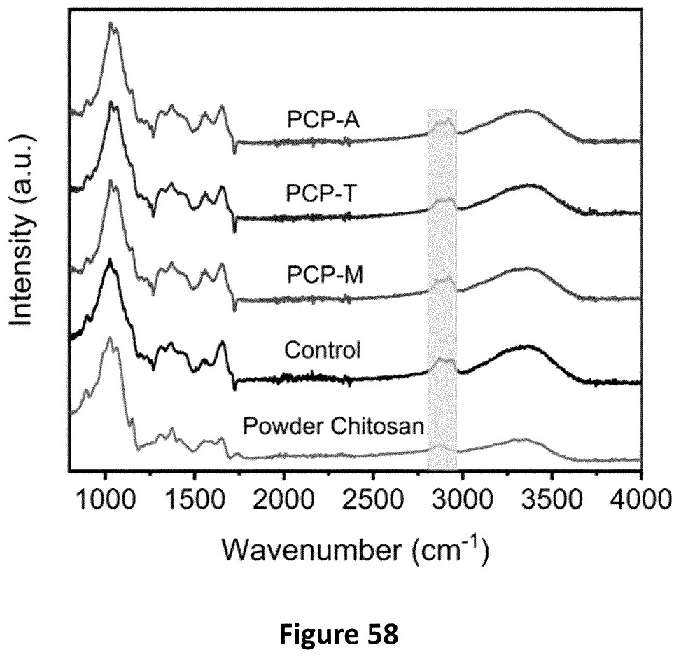

FTIR: FIG. 58 shows the infrared spectrum of chitosan. A strong band in the region 3291-3361 cm⁻¹ corresponds to N—H and O—H stretching, as well as the intramolecular hydrogen bonds. The absorption bands at around 2921 and 2877 cm⁻¹ can be attributed to C—H symmetric and asymmetric stretching, respectively. These bands are characteristics typical of polysaccharides and are found in other polysaccharide spectra, such as xylan, glucans, and carrageenan. The peak for powder chitosan and PCP-M around 2921 and 2877 cm⁻¹ had lower intensity indicating similar stretching, whereas PCP-A and PCP-T had higher intensity bands. The presence of residual-acetyl groups was confirmed by the bands at around 1645 cm⁻¹ (C—O stretching of amide I) and 1325 cm⁻¹ (C—N stretching of amide III), respectively. The band corresponding to the N—H bending of amide II was missing at 1550 cm⁻¹ in powder chitosan indicating possible overlap with other bands, but a small band was observed in the synthesized porous samples. The bands at 1423 and 1375 cm⁻¹ confirm the CH₂ bending and CH₃ symmetrical deformations, respectively. The absorption band at 1153 cm⁻¹ and 1066 and 1028 cm⁻¹ can be attributed to the asymmetric stretching of the C—O—C bridge and C—O stretching, respectively. All bands are found in the spectra of samples of chitosan reported previously.

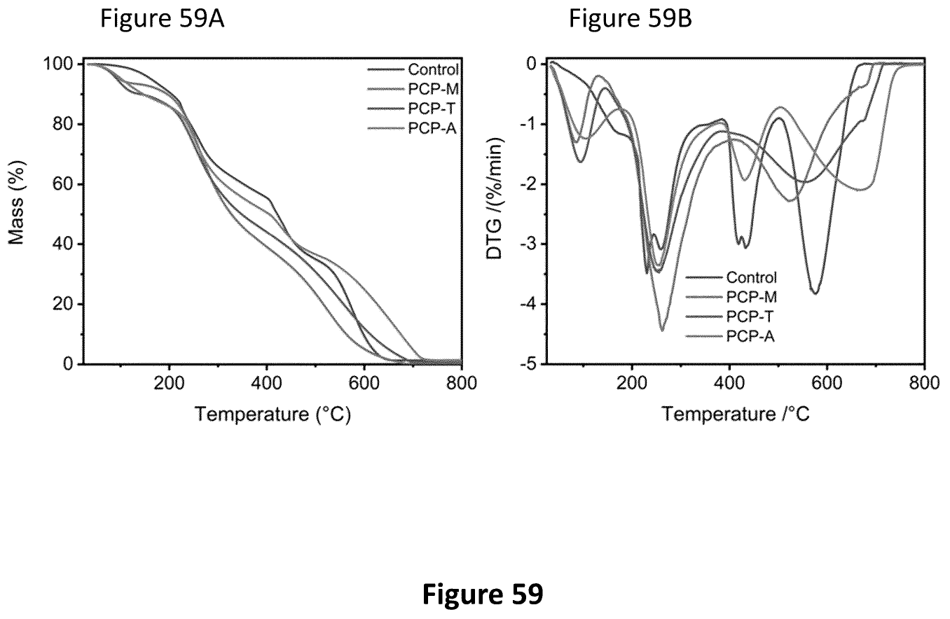

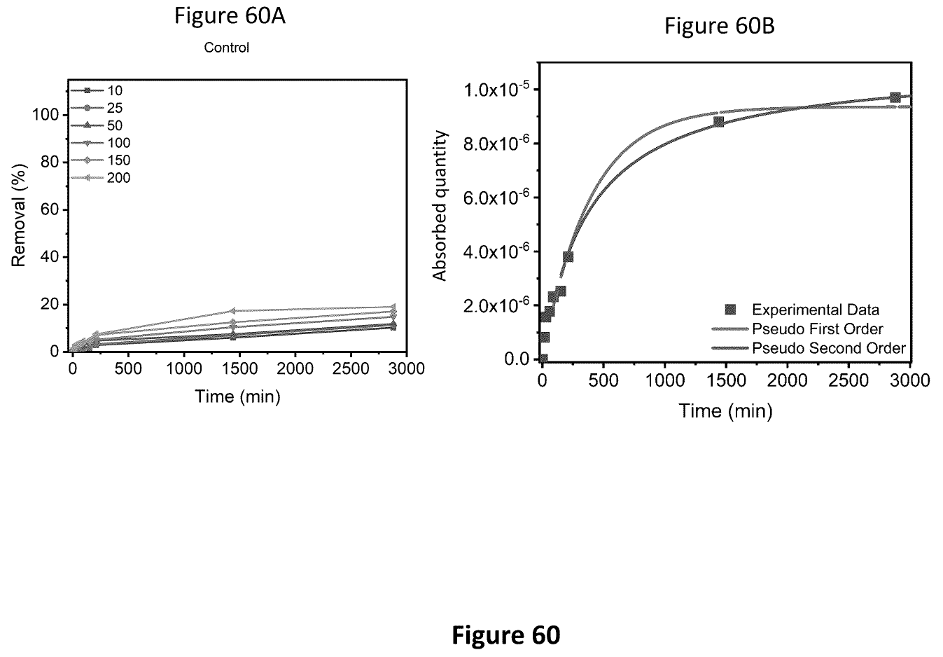

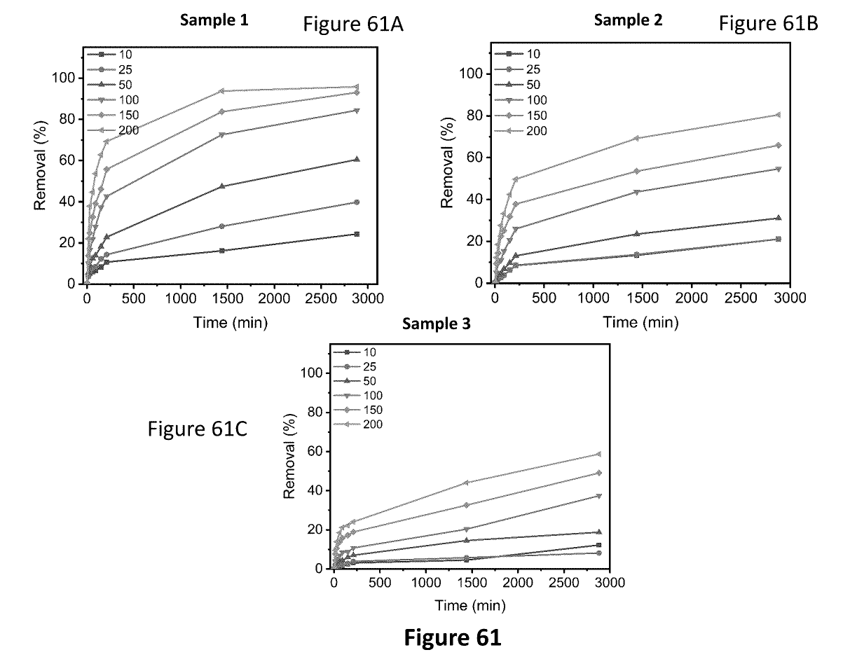

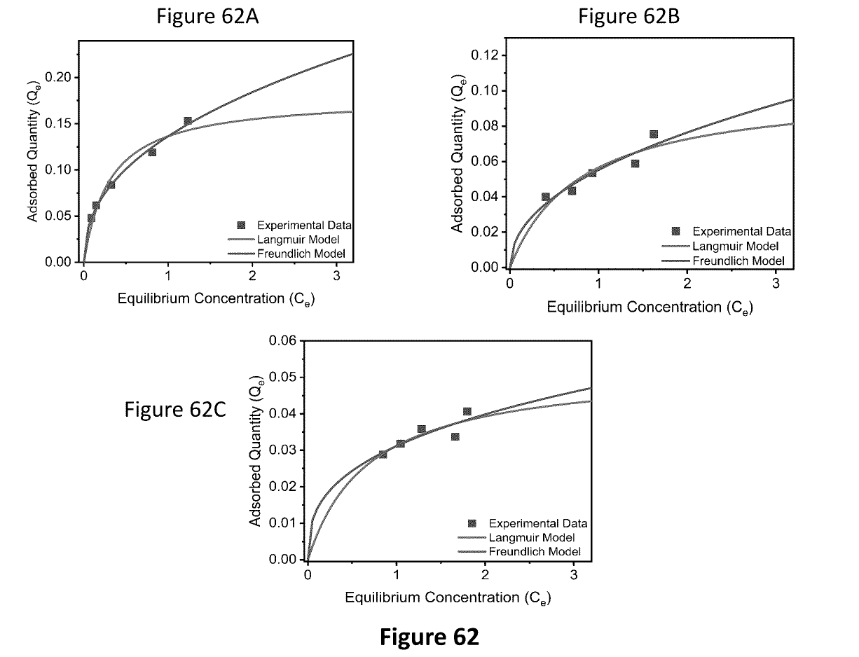

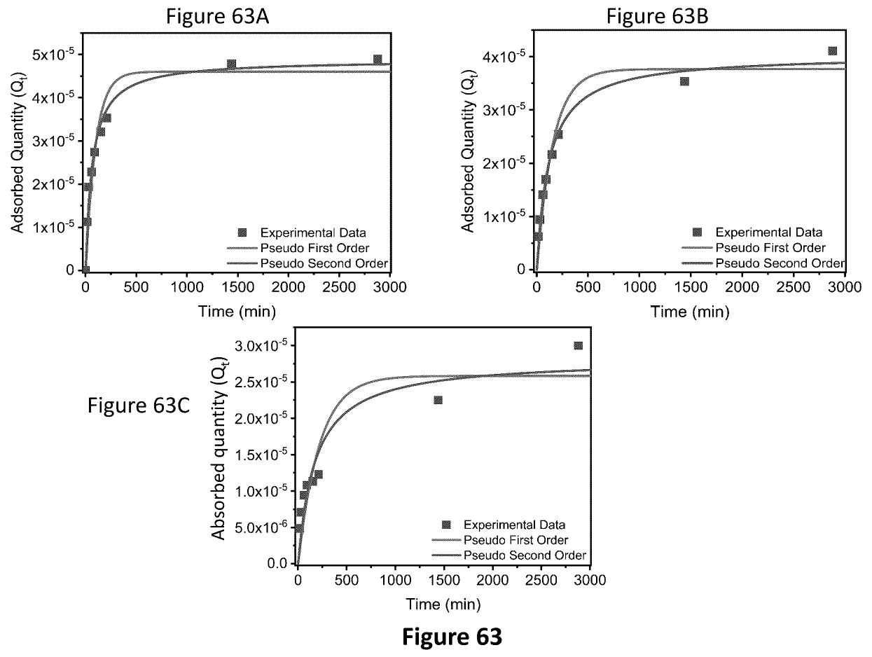

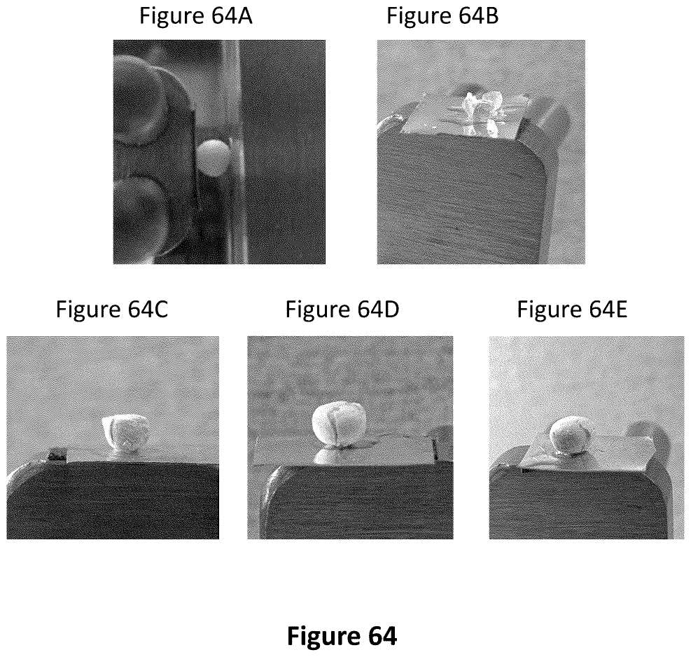

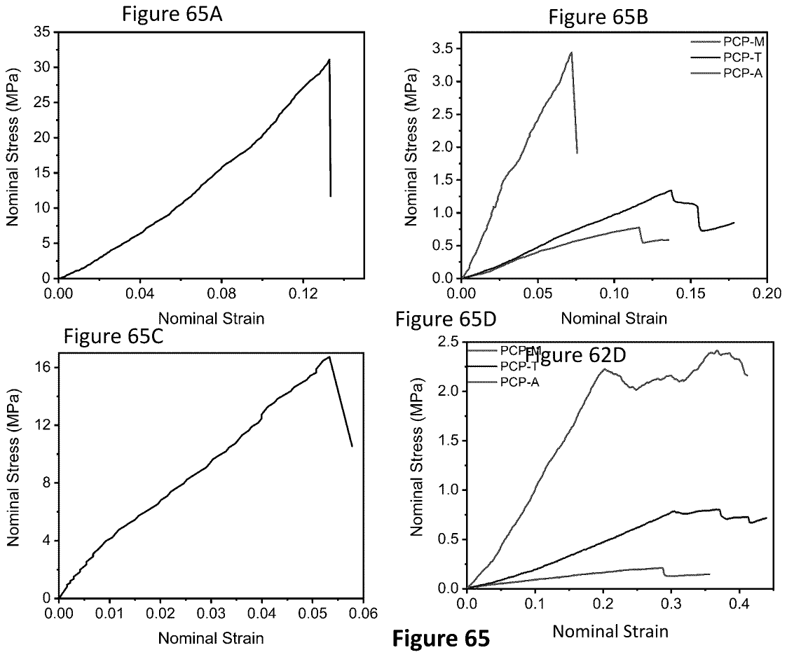

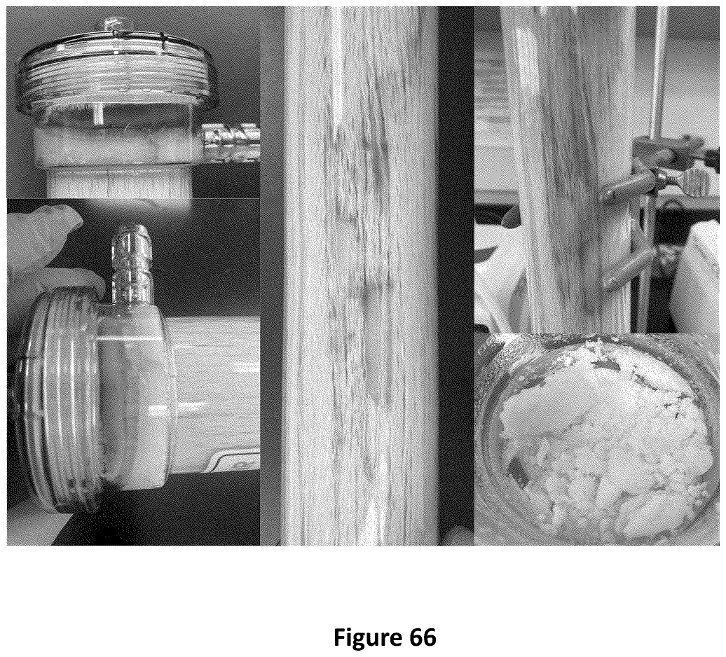

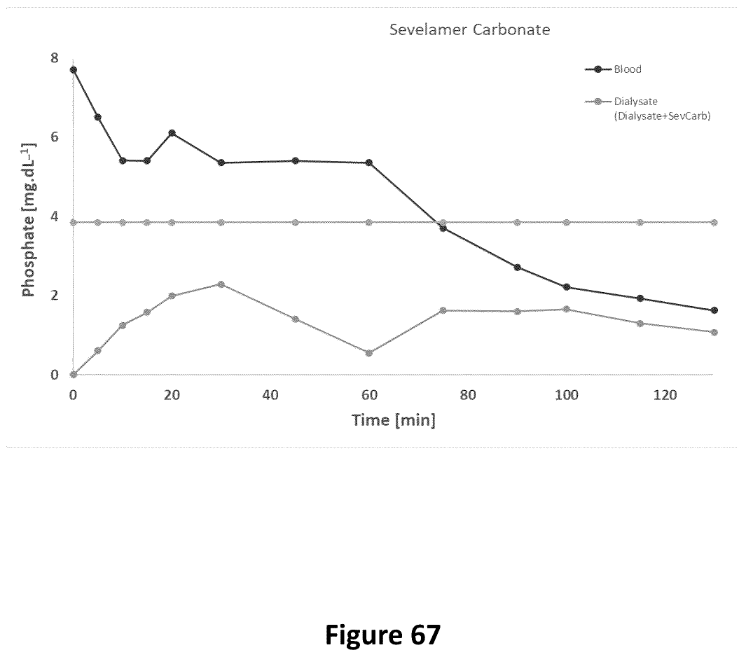

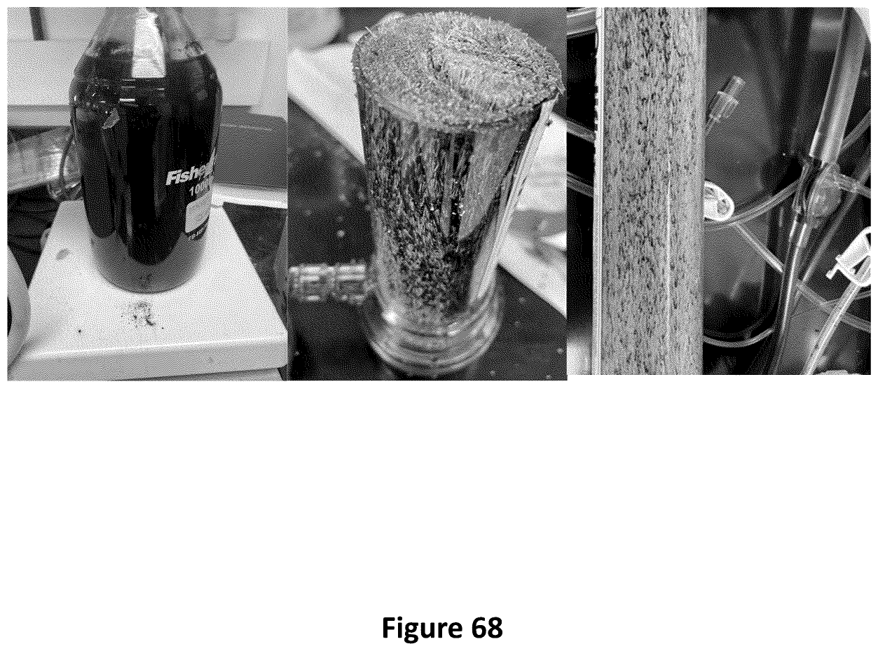

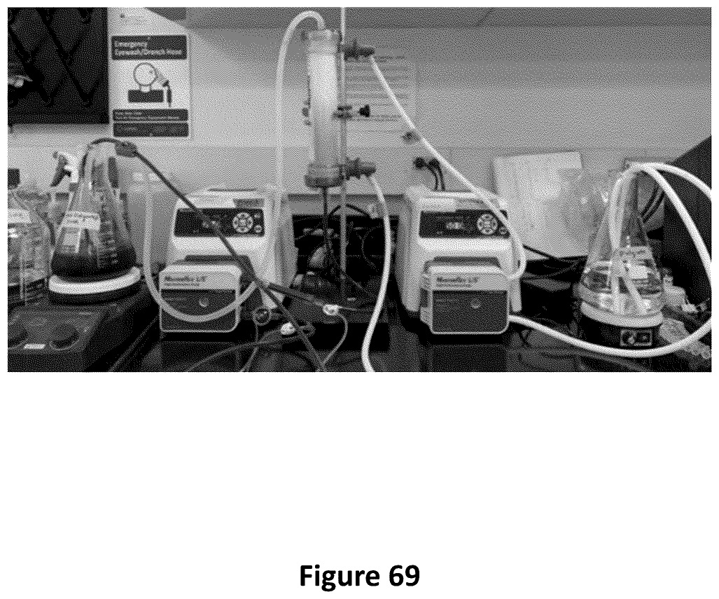

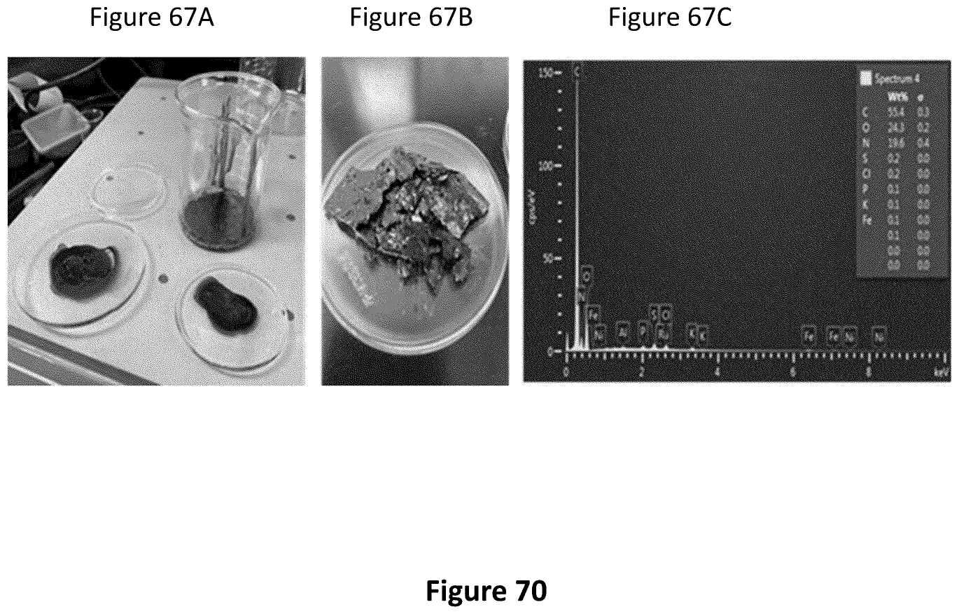

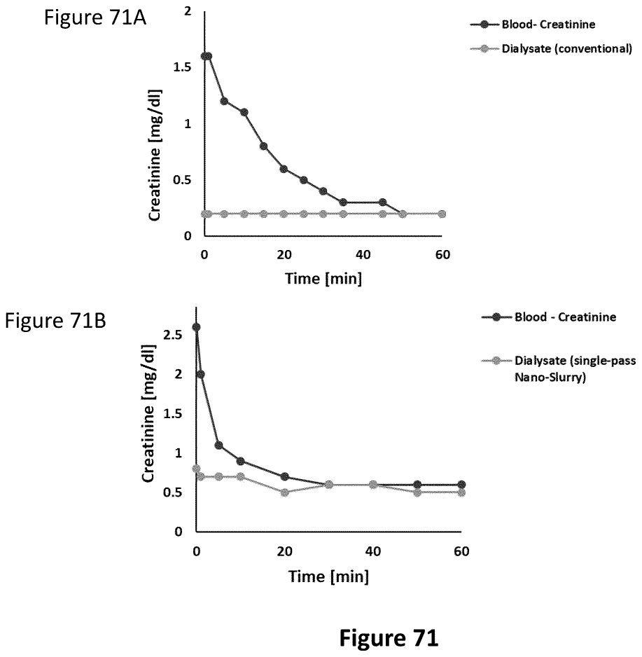

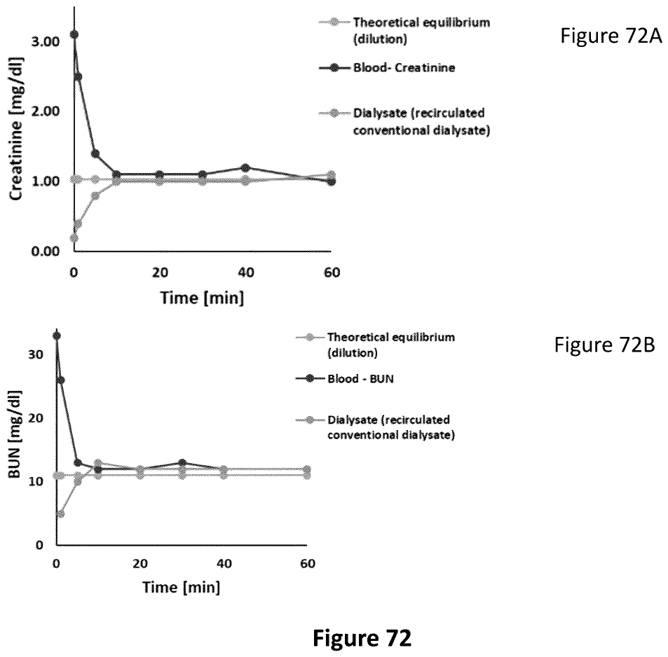

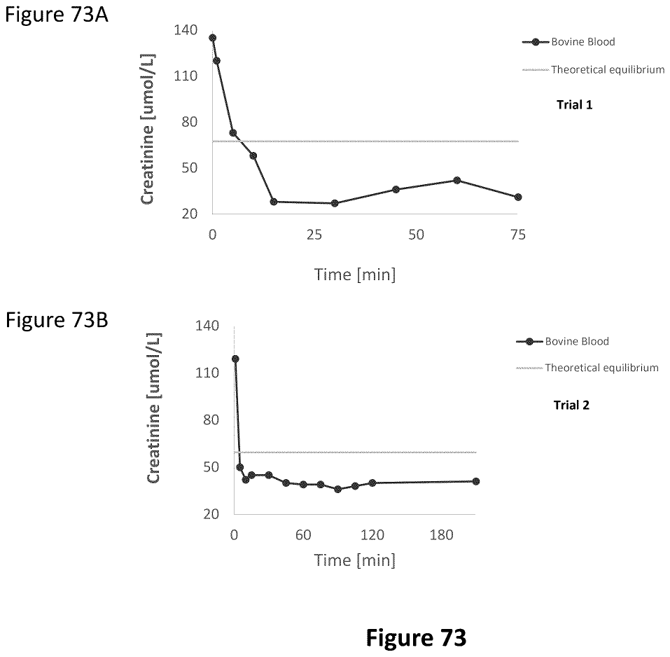

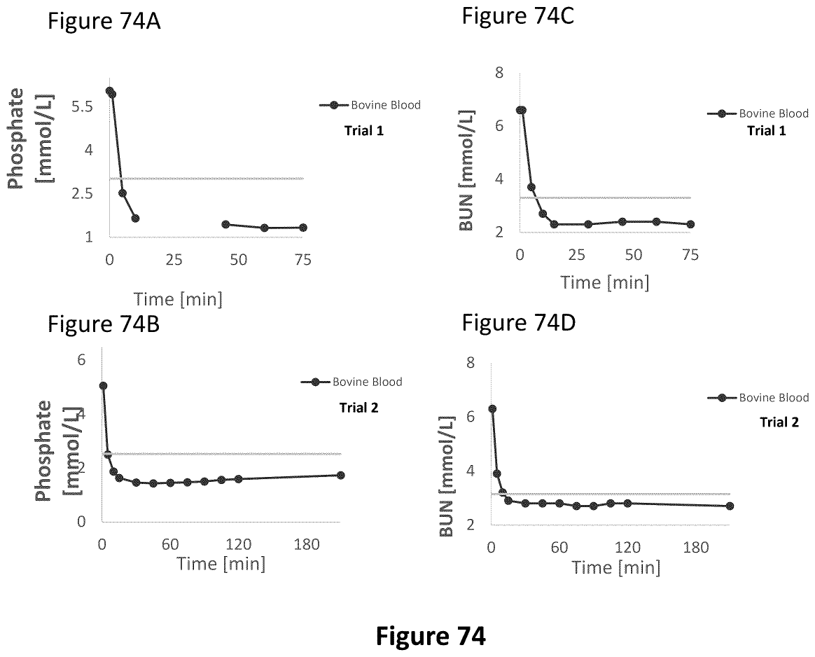

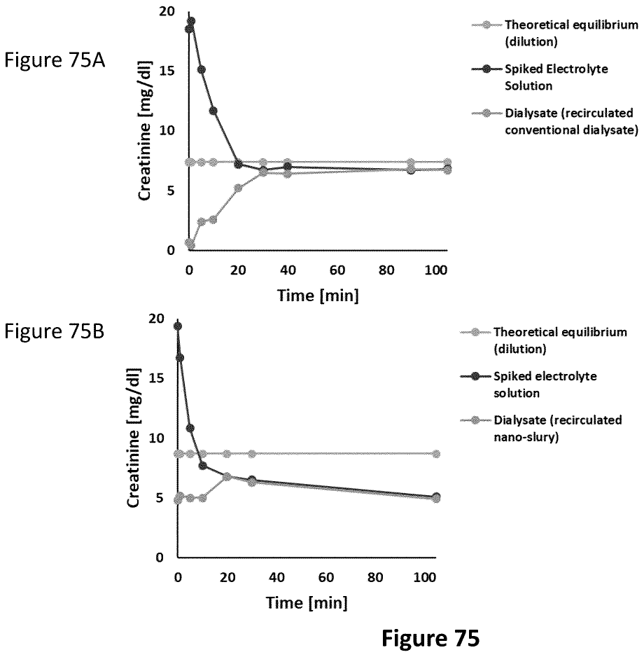

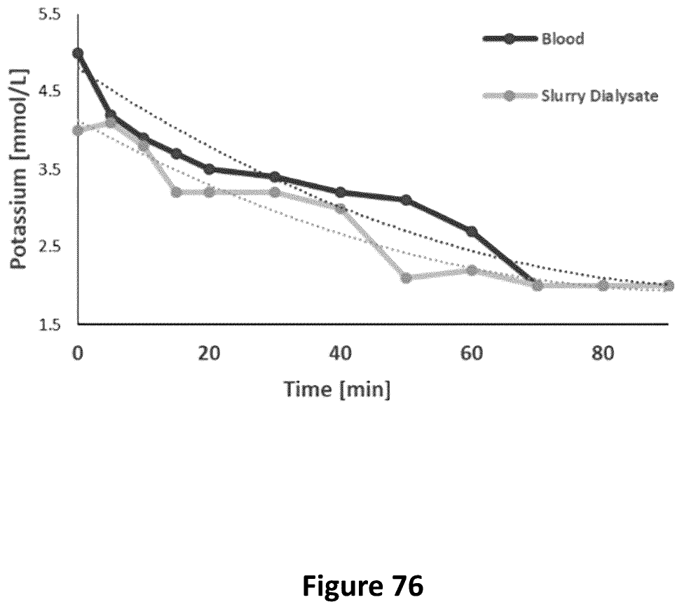

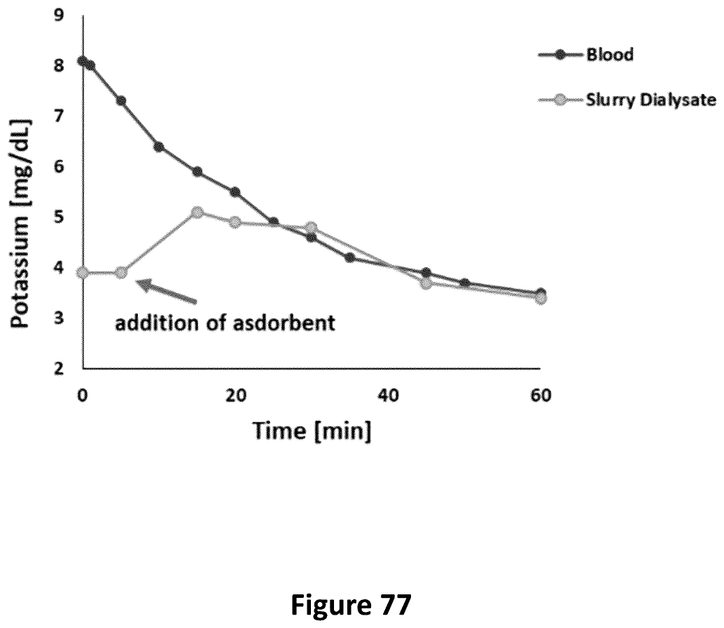

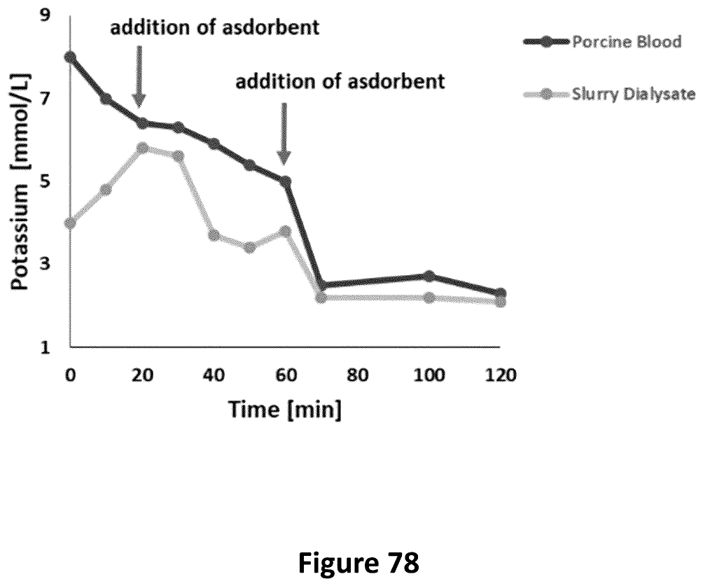

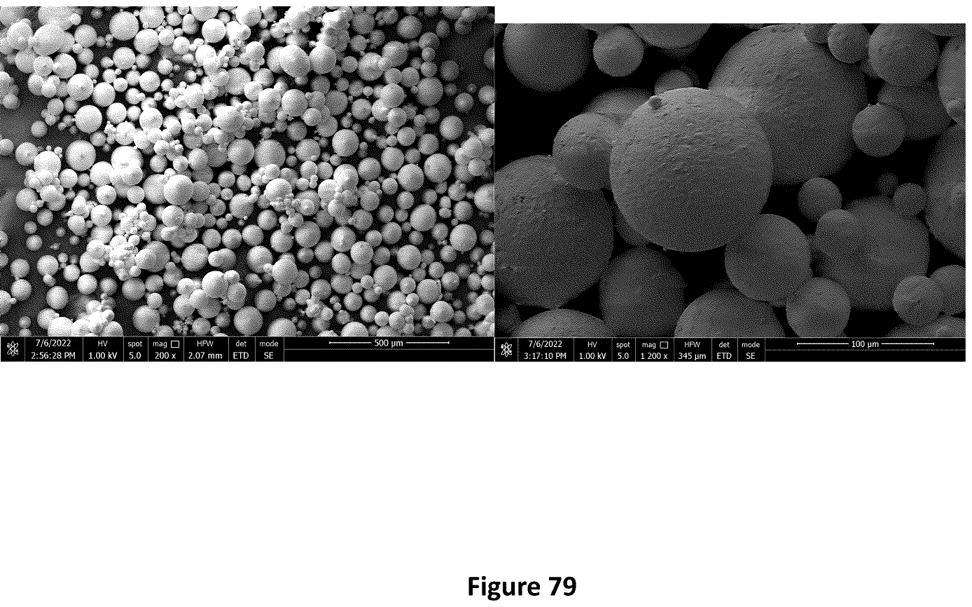

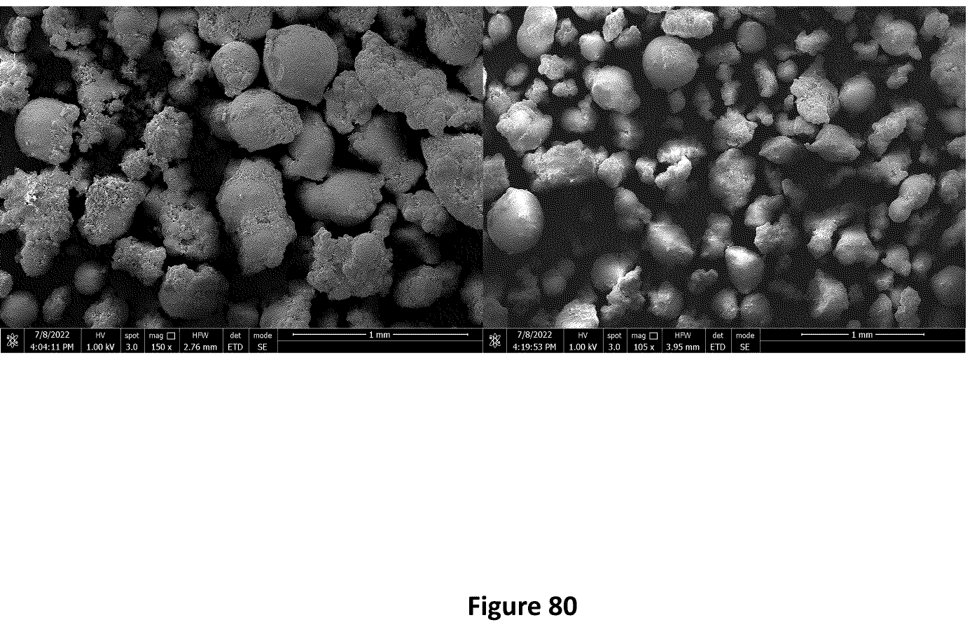

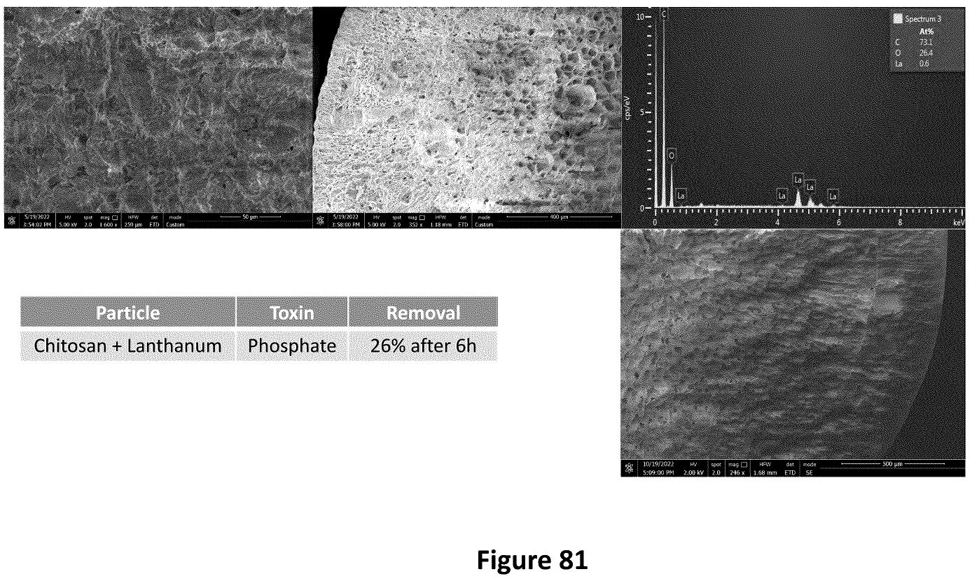

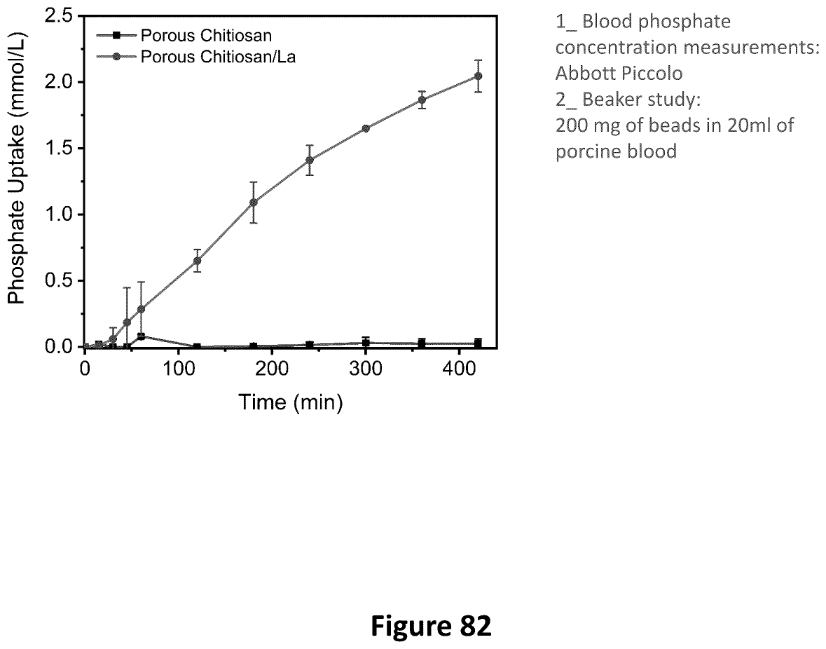

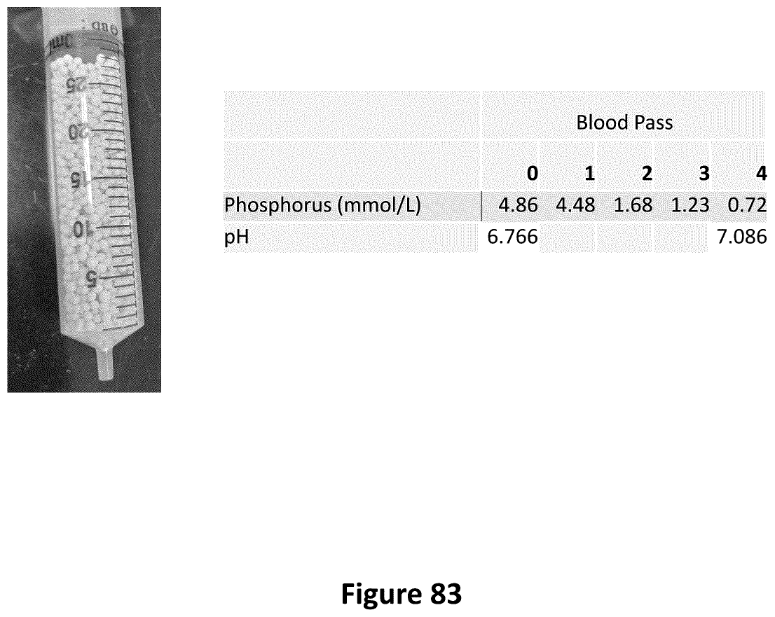

TGA: The TGA curves of chitosan particles exhibit weight loss in three stages as shown in FIG. 59. The first stage appeared in the range of 40-100° C. which can be attributed to the water loss. The water weight loss is around 0.9% for the control sample while it is significantly higher for the porous particles around 6-11%. The second degradation is around 280° C. A change of the thermogram curve at 430° C. suggests a slower third process. Another process was observed in the derivative curve between 550-650° C. The correspondent derivative curve reached zero between 650-800° C. with no evident distinct events. The FTIR analysis conducted on the samples previously demonstrated the presence of certain peaks corresponding to certain functional groups.

Previous research has reported that evolved gas mainly composed of water, ammonia, carbon monoxide, carbon dioxide, acetic acid, and methane is released during the thermal degradation of chitosan. A main process involving the release of water, ammonia, carbon monoxide, carbon dioxide, and acetic acid is assigned to the pyrolytic degradation of chitosan, as well as assessed in the literature, in the temperature range 250-450° C. In the temperature range of 250-450° C. a weight loss of around 35%. was observed, possibly corresponding to this gaseous release. At lower temperatures, the release of ammonia release is initiated which reaches its peak at 325° C., suggesting a low activation energy for ammonia formation. A second process characterized by the release of methane possibly happened in the range 550-650° C. corresponding to a loss of around 30%. The highest rate of methane generation is observed at around 590° C. in control chitosan, as observed in the previous studies. The synthesized microspheres have a deviation in the peak which occur between 550-650° C. with the maximum generation of CH4 observed around 550, 580, and 650° C. for PCP-M, PCP-T, and PCP-A, respectively. A modification of the material is suspected after the complete reduction of the structure causing methane production and the consequent formation of a graphite-like structure via the dehydrogenation mechanism, as suggested in the literature. Monitoring the evolution of species in temperature/time scale is imperative to identify a complex degradation pattern.

Adsorption Isotherms and Kinetics: The methylene blue removal in samples was significant in synthesized porous microspheres compared to the non-porous microsphere. The uptake in porous microspheres increased with time before reaching equilibrium. The removal exceeded 90% at the equilibrium value after 1440 minutes, rapidly increasing after the initial 120 min, indicating fast adsorption. The non-porous microspheres showed the lowest equilibrium removal, possibly due to decreased accessibility for mass diffusion and lack of a porous network. PCP-M showed the highest qe and k2, consistent with its high uptake and removal rate.

The isotherms were derived using the Freundlich and Langmuir models. The isotherm plots of the Freundlich and Langmuir models are shown in FIG. 60. The Freundlich model fitted the data better, with higher correlation coefficients, indicating that the dye removal followed reversible multilayer adsorption by the heterogeneous surface of the microsphere. Furthermore, the Freundlich equation predicts that the dye concentrations on the adsorbent will increase with an increase in the dye concentration in the liquid phase.

The adsorption kinetics of the microspheres were investigated. The pseudo-first order and the pseudo-second-order kinetic models were applied to analyze the adsorption kinetics. The pseudo-second-order kinetic model was more suitable for the overall adsorption process (after 2880 min), with higher correlation coefficients (R²>0.98) than those of the pseudo-first-order kinetic model (R²<0.98), indicating the primary mechanism of chemical adsorption. The fitted curves by this model are shown in FIG. 60 The PCP-M microspheres showed a higher equilibrium removal qe and a lower rate constant k, possibly due to improved accessibility for mass diffusion.

Mechanical Properties: The mechanical properties of the microspheres were obtained by analyzing the force-distance curves centered on the particle. FIG. 61 shows the setup of the microspheres in the MicroTesting unit. Finally, 4 effective curves for the synthesized porous and non-porous microspheres of each size were used to plot stress and strain. During the compression, the shape of the particles changes continuously. Therefore, nominal stress and strain were used instead of stress and strain. The nominal stress (calculated by the applied force divided by the cross-sectional, as a function of nominal strain (the displacement divided by the diameter of particles) was measured to assess the elastic properties of the chitosan microspheres.

Young's modulus is determined based on uniaxial tension or compression. To measure Young's modulus of a particle the effective modulus of the particle must be determined using the equation below:

$$F = \frac{2}{3} {E}^{*} \sqrt{r \times {δ}^{\frac{3}{2}}} \qquad ( 1 )$$

F, d, and δ are applied force, particle diameter, and total displacement. Young's modulus can be calculated from the effective modulus:

$${E}^{*} = E / 1 - {v}^{2} \qquad ( 2 )$$

v is the position ratio of the particles. The Poisson ratio of the particles is unknown. Therefore, the slope of the nominal stress and strain was determined as deformation resistance. Deformation resistance is equivalent to the Young Modulus, and it can be considered an inherent property of the materials.

FIG. 62 presents the stress vs. strain plot used to determine the deformation resistance (DR) of the synthesized material. The materials properties, morphology, and roughness of the chitosan microspheres influence the deformation resistance determined from the slope of the stress versus strain plot. Table 9 summarizes the deformation resistance and yield strength of the microspheres and linear part of the nominal stress and strain slope was used to determine the deformation resistance. The non-porous chitosan microspheres had the highest deformation resistance (235.43±11.34 MPa), indicating very low elasticity. PCP-M demonstrated the highest elasticity with DR of 7.93±0.72 MPa, followed by PCP-T (9.95±0.16 MPa). PCP-A had the lowest elasticity and highest YM of 40.92±23.79 MPa among the porous chitosan microspheres. After soaking the microsphere in water for 24 hours, the elasticity of the particles changed significantly. The deformation resistance of PCP-M (0.73±0.04) was the lowest, followed by PCP-T (2.63±0.16 MPa) and PCP-A (15.66±5.05 MPa). The elasticity of PCP-M is the lowest number in wet and dry conditions, which confirms that the particles are softer after introducing the porosity to the structure. Interestingly, the deformation resistance of the non-porous microsphere did not change drastically from non-soaked particles (304.06±36.31 MPa).

TABLE 9
Comparative summary of deformation resistance and compressive strength of the
microsphere before and after 24 hour soaking in water.

| | Deformation resistance (MPa) | | Compressive strength (MPa) | |
|---|---|---|---|---|
| Microsphere | Non-soaked | Soaked | Non-soaked | Soaked |
| Non-porous | 235.43 ± 11.34 | 304.06 ± 36.31 | 31.55 ± 0.61 | 19.94 ± 4.54 |
| PCP-M | 7.93 ± 0.72 | 0.73 ± 0.04 | 0.76 ± 0.03 | 0.19 ± 0.02 |
| PCP-T | 9.95 ± 0.16 | 2.63 ± 0.16 | 1.29 ± 0.07 | 0.78 ± 0.12 |
| PCP-A | 40.92 ± 23.79 | 15.66 ± 5.05 | 3.52 ± 0.11 | 2.26 ± 0.40 |

In this study, three distinct synthesis methods were employed, utilizing environmentally friendly solvents and crosslinkers. The results showed that it is possible to tailor the beads' average pore diameter and total pore volume to suit the target molecules. However, pore volume and diameter alterations were observed to induce changes in a particle's surface area. As a result, optimizing selectivity for a specific range of toxin molecular weights could inadvertently diminish the efficacy of the adsorbent and ion exchange resins by reducing their overall surface area. Understanding the mechanical strength and structural behavior of polymeric beads, especially in their wet state when they undergo swelling, is of utmost importance. When wet, these beads swell, significantly altering their structural integrity and performance. If the beads are mechanically weak, they can rupture or deform under stress, compromising their function. Maintaining structural resilience in the swollen state ensures operational longevity and effectiveness. Therefore, thoroughly understanding and optimizing their mechanical strength in this state is critical (Lin, D., et al., 2009, Biomech. Model. Mechanobiol., 8 (5), 345; Lee, D., et al., 2019, Elastic Mod. Meas. of Hydrogels, 865-884; Perspectives: Strengthening The Effectiveness Of Minority-Serving Institutions| Diverse: Issues In Higher Education). Here, the mechanical properties of the microspheres were obtained by analyzing the force-distance curves centered on the particle.

Therefore, nominal stress and strain were used instead of stress and strain. The nominal stress (calculated by the applied force divided by the cross-sectional) and the displacement divided by the diameter of particles were measured to assess the elastic properties of the chitosan microspheres. Young's modulus was determined based on uniaxial tension or compression (Brady, J. E., and During, T., 2009, Devel. Solid Oral. Dos. Forms: Pharm. Theory and Prac., 187-217; Lin, D., et al., 2009, Biomech. Model Mechanobio., 8 (5), 345; Lee, D., et al., 2019, Elastic Mod. Meas. of Hydrogels, 865-884; Chang, C., et al., 2018, Advances in Mat. Sci. and Eng., 2018; Schneider Y., et al., 2023, Polymers (Basel), 15 (7)). The two types of beads, control and macroporous, exhibiting the lowest adsorption performance, remarkably presented the highest mechanical strength when in a wet state. Therefore, optimizing the performance of chitosan beads is imperative. Nonetheless, achieving this optimization necessitates systematic studies that delve into a more profound comprehension of the biopolymers' behavior at the molecular level.

The FTIR and XRD show the presence of the chitosan phase and characteristic peaks for the chitosan structure. The comparative analysis with pristine chitosan demonstrated that the porous microspheres retained the functional and structural phases of the chitosan through the synthesis process. The SEM and Fluorescent imaging highlighted the porous network in the synthesized microspheres and the lack thereof in the non-porous chitosan. The pore sizes differ significantly in the PCP-A, PCP-M, and PCP-T samples, which are reflected in the properties studied in this research. The largest pore size was observed in PCP-A, followed by PCP-M and PCP-T microspheres. The adsorption kinetics, isotherms, and elastic properties of these microspheres indicate that PCP-M is the higher-performing microsphere. The optimum pore size of the PCP-M microsphere allows higher and faster adsorption, better elasticity, and structural integrity.

The large pore size of PCP-A reduces the adsorption and elasticity of the microsphere. Similarly, the small pore size of PCP-M microsphere induces lower adsorption, possibly due to lower contact area and restricted movement inside the porous network. The elasticity of the microsphere is also reduced. The porous structures offer superior adsorption benefits compared to bulk structures. However, it's crucial that these porous particles maintain their structure when wet. As anticipated, introducing porosity significantly reduces the particles' compressive strength. Their stress-strain curve can be divided into two phases: an initial linear elastic stage and a densification region. The PCP-T, with its densely packed morphology as confirmed by Brunauer-Emmet-Teller (BET) and SEM images, exhibits the highest compressive strength. This tightly packed structure combined with microporosity results in enhanced mechanical properties.

### Example 5: Nanoslurry

Improving the efficacy and efficiency of dialysate for hemodialysis has the potential to improve patient outcomes and reduce waste and energy consumption. Currently, hemodialysis relies mostly on diffusive clearance across a gradient between the patient's blood and the dialysate. The addition of nanoparticle adsorbents to the dialysate to improve the diffusive efficiency of hemodialysis was shown. The reuse of dialysate was also shown.

This is an in-vitro hemodialysis experiment using Li-heparinized porcine blood, two peristaltic pumps, Fresenius Optiflux hemodialysis membranes, and CitraPure dialysate concentrate (Rockwell Medical) which was diluted and the pH was adjusted using medical-grade sodium bicarbonate (Solcart B, B. Braun). One liter of freshly collected porcine was dialyzed with an average blood flow rate of 30 mL/min and dialysate flow of 50 mL/min. Nanoscale activated carbon (Nano-Slurry) was tested in this study. The mass of added adsorbents (0.5 to 5 g/l) varied depending on the adsorbent type, targeted blood toxin, and expected removal rate. Absorbent leaching to blood was tested by using highly concentrated Nano-Slurry (10 to 20 g/l) dialysate and increased transmembrane pressure by increasing dialysate flow rate to 1000 mL/min and measuring the reverse filtrate for nanoparticles by turbidity with a near-infrared turbidity meter.

In the single pass experiment, creatinine was removed in ½ much as time. In the recirculated dialysate experiment with the Nano-Slurry, creatinine rapidly equilibrated in the control experiment at 30 minutes whereas, the recirculated Nano-Slurry dialysate continued to remove creatinine at the end of the experiment, 100 minutes. Blood urea nitrogen rapidly lowered from 17 mg/dL to 7 mg/dL, below the calculated equilibrium at 20 minutes. For the absorbent leaching experiment, the reverse filtrate had turbidity that remained unchanged pre- and post-experiment with a measure of 0.03 NTU.

Nano-Slurry improved the efficiency in both in-vitro single pass and recirculated hemodialysis models. The addition of nanoscale adsorbents improved the efficiency of dialysis and can be incorporated into a novel hemodialysis machine that can recirculate dialysate, reducing water consumption for both in-center and home hemodialysis.

Very little research has focused on sorbent technologies, especially hybrid techniques in which sorbent has been added to the dialysate. One study used activated charcoal to bind middle molecules. No studies have examined whether charcoal can also increase the removal of potassium and phosphorus. The removal of potassium is of potassium is important as large gradients and the rapid removal are associated with cardiac arrythmias. The removal of phosphate is of clinical significance as most phosphate is removed within the first hour of hemodialysis and improved phosphate removal on hemodialysis can reduce the significant pill burden of phosphate binders.

In this proof of concept study, adsorbent nanoparticles were added to an in-vitro hemodialysis to improve the removal of electrolytes and small molecules in hemodialysis in a single pass and a recycled dialysate model. Commercially available potassium binders (sodium zirconium cyclosilicate, ZS-9) and phosphate binders (sevelamer carbonate, SC) were studied to see if they can be added as sorbents to remove potassium and phosphate in an effective, efficient, and controlled manner. Insights from this study, is the first step in improving the effectiveness and efficiency of hemodialysis, with the goal of improving incomes with precision medicine, developing home and wearable technologies, while reducing the environmental impact. For a better visual representation of using polymeric gels added in dialyzers, FIG. 63 shows polymeric gels with an excess dose of sevelamer carbonate in a dialyzer. FIG. 64 shows results from an early proof of concept experiment depicting precision treatment using sevelamer carbonate with dialysate.

Experimental design: An array of nanomaterials and composites are synthesized and tested in this study. The mass of added adsorbents (0.5 to 5 g/l) varies depending on adsorbent type, targeted blood toxin, and expected removal rate. Control and slurry (adsorbent(s) added to dialysate) hemodialysis experiments were performed using porcine blood. Lithium heparin is added to the blood as an anticoagulant at the time of collection by a certified vendor. Porcine blood is shipped on ice and is tested for hemolysis before the experiment. All experiments are conducted upon the arrival of blood. Commercially available dialysate (cleansing solutions) and dialyzers (filtration unit) are used in the studies. Fresenius Optilfux dialyzers (F160NR and F250NR) and CitraPure dialysate concentrates (Rockwell Medical) were used to prepare dialysate. Dialysate pH is adjusted by adding pharmaceutical-grade sodium bicarbonate (Braun Avitum). Generally, the porcine blood has an elevated phosphate concentration (5.5±0.5 mmol/1) compared to expected phosphate concentration range in human blood (1.25 to 2.10 mmol/l). Thus, no phosphate is added to porcine blood to mock hyperphosphatemia in the experiment. Potassium concentration varies depending on the shipping time (7 to 9 mmol/l). The expected potassium concentration range in human blood is 3.4 to 5.5 mmol/l. Thus, the elevated potassium levels in porcine blood were used to demonstrate hyperkalemia (potassium concentration above 5.5 mmol/l).

Benchtop mock hemodialysis studies: A series of photos depicting a dialysate slurry and dialyzer are shown in FIG. 65. Two peristaltic pumps (Masterflex L/S, Cole-Palmer) and a dialyzer are used to run bench-scale studies, as shown in FIG. 66. Fresenius Optiflux dialyzers (F160NR and F250NR) and CitraPure dialysate concentrate (Rockwell Medical) were used. The CitraPure dialysate solution is diluted and pH is adjusted using medical-grade sodium bicarbonate (Solcart B, B. Braun). Initially, experiments to achieve an equilibrium between the dialysate and blood through transmembrane diffusion were performed. Thus, flow rates and consequently transmembrane pressure are adjusted accordingly to avoid ultrafiltration. Once kinetic diffusion studies are accomplished at equilibrium, the next step was adding convective mass transfer; by adjusting the flow rates and transmembrane pressure. Therefore, flow rates were adjusted as such that fluid removal (ultrafiltration) can be achieved during mock studies.

Ex vivo hemodialysis: A Dialog B. Braun (B. Braun Avitum AG) hemodialysis machine was used for studies. Flowrates were adjusted to maintain a 10 mL/h/kg ultrafiltration rate. 6 liters of freshly collected porcine were dialyzed with an average blood flow rate of 400 mL/min and dialysate flow of 600 mL/min. All hemodialysis experiments were isothermal and conducted at 37±1° C.

Adsorbent Leaching to Blood: Multiple methods were developed to test the leaching of nanoslurry (nanoparticles) in the blood. In one approach, the dose of adsorbents in dialysate was increased by 200% and extended the hemodialysis duration to 5 hours. A higher concentration of nanoparticles and an increased duration of hemodialysis raise the possibility of particles passing through the membrane. After completing a 5-hour hemodialysis treatment with an elevated dose (20 mg/l) of adsorbent in dialysate, multiple blood samples were collected. In this study, 1 liter of porcine blood was dialyzed with 1 liter of nanoslurry dialysate. 100 ml blood was collected samples from the top, middle, and bottom of the blood container. Samples were centrifuged at 13,000 RPM using a benchtop Sorvall™ centrifuge (30 to 60 mins) and oven-dried overnight at 80° C. The dried residuals were analyzed using energy dispersive X-ray spectroscopy (EDS) analytical equipment for elemental analysis as shown in FIG. 67. In addition, blood pH, electrolytes, and metabolic composition were continuously monitored throughout the mock-dialysis treatments. Blood samples were tested for elemental analysis using inductively coupled plasma mass spectrometry (ICP-MS) to test for composition of the nanoadsorbents in blood due possible leaching.

In another series of experiments, highly concentrated nanoslurry (10 to 20 g/l) dialysate was filtered using Fresenius dialyzers. In this method, a transmembrane pressure was applied that was in the opposite direction of a hemodialysis treatment; thus, the nanoslurry was forced against the hollowfiber membrane to see if the suspended particles pass through the membrane as preliminary measure of safety. The water samples were tested for turbidity using a turbidity meter (LaMotte 1947-1). Additionally, water samples were tested using an inductively coupled plasma mass spectrometry (ICP-MS) for elemental analysis to see if elements such as zirconium, lanthanum, or other elements of the adsorbents have released to the blood side.

Creatinine removal in single-pass mock dialysis: In the first experiment, adding nanoscale activated carbon (NAC) in conventional dialysate solution to increases the removal rate of creatinine from 1 liter of porcine blood in single-pass benchtop mock dialysis was examined. Here, creatinine (113 Daltons) was used as a small model molecule to test the Nano-Slurry concept. To further demonstrate the efficacy of the NAC creatinine was added to the slurry-dialysate solution (0.8 mg/dl). Then 1 liter of porcine blood, blood creatinine concentration (2.6 mg/dl), was dialyzed by 2 liters of Nano-Slurry (NAC) dialysate in a single-pass mode. When 1 liter of porcine blood is dialyzed with 2 liters of dialysate, it was expected for the analytes to be diluted three times due to diffusing at equilibrium. FIG. 68 shows the results of this experiment compared to a control experiment of commercial dialysate without creatinine (note that 0.2 mg/dl is limit of detection)

In the control experiment, creatinine was gradually removed over 60 minutes of single-pass dialysis. The creatinine concentration was reduced by half (0.8 mg/dl) within the first 15 minutes of dialysis. In the single-pass Nano-Slurry (NAC) experiment, the creatinine concentration was reduced from 2.6 to 1.1 mg/dl in the first 5 minutes of the treatment compared to the control experiment in which creatinine concentration was reduced by half (0.8 mg/dl) within the first 15 minutes of dialysis.

Recirculation of dialysate: Impact of dilution and flow rates on benchtop studies. Here, conventional dialysate was recirculated to dialyze porcine blood with an initial creatinine concentration of 3.1 mg/dl, and 1 liter of porcine blood was dialyzed by 2 liters of dialysate. Blood and dialysate flow rates were increased by 200% compared to the single-pass dialysis study. Similar rapid removal trends were observed as those obtained from the single-pass Nano-slurry experiment. The creatinine is rapidly removed from the blood and reaches an equilibrium with dialysate within the first 10 minutes of the dialysis. As shown in FIG. 69, adding nano adsorbents and increasing the flow rates could lead to similar clearance trends. Thus, in benchtop mock hemodialysis studies, dialysate and blood flow rates and volumes must be kept consistent throughout comparative studies to prove the impact of adsorbents correctly. Therefore, the effect of flow rates, ultrafiltration rate, and dilution factor should be carefully monitored and considered in reporting removal rates and adsorption capacities of Nano-Slurry dialysate. FIG. 70 depicts depicting albumin and creatinine removal at high flow rates (300/400 ml·min⁻¹). FIG. 71 shows phosphate and blood urea nitrogen management.

Recirculation of Dialysate with the Addition of Adsorbents: To test the Nano-Slurry concept and check the removal capacity of the design, a 0.5 L of dialysate solution with 4.5 g/l of NAC was recirculated to dialyze 0.5 L electrolyte solution with an excess amount of creatinine (20 mg/dl) using a Fresenius F160NR dialyzer in benchtop mock dialysis experiments. As shown in FIG. 72, the control experiment shows creatinine dilution by recirculation of conventional dialysate reaching concentrations slightly lower than the theoretical equilibrium concentration after 30 minutes. In the NAC experiment, 5 mg/dl of creatinine and 4.5 g/l of NAC was added to the dialysate, creatinine was more rapidly removed than in the control experiment and did not reach equilibrium at 100 minutes.

Nanoslurry of potassium management: Next was examined whether absorbents added to the dialyzer can still effectively lower potassium while minimizing blood to potassium gradient. 1 L of porcine blood with an initial potassium concentration of 5 mmol/L was dialyzed using a slurry dialysate with a potassium concentration of 4 mmol/L. A shown in FIG. 73, the serum potassium level was reduced to low levels (2 mmol/l) to showcase the efficacy of the nanoslurry concept; selective removal of targeted ions and molecules to offer precision medicine. Thus, the following examples were presented to showcase precision medicine in potassium management. Initial and final serum potassium levels are exaggerated to demonstrate the capabilities of the nanoslurry approach.

In the next experiment, severe hyperkalemia was mocked, starting with porcine blood with a significantly elevated serum potassium level (8 mmol/l) and dialyze it against a nanoslurry dialysate with a potassium concentration of 4 mmol/L. As shown in FIG. 74, after 60 minutes of dialysis and one dose of nanoslury added after 5 minutes of dialysis, the serum potassium concentration was gradually lowered to a final concentration of 3.3 mmol/l. Once the adsorbent was added to the recirculating conventional dialysate, as shown in FIG. 74, the concentration gradient was decreased to a minimal level, and potassium was removed from serum and blood simultaneously, allowing a gradual removal of potassium throughout the treatment.

In the next severe hyperkalemia experiment, sequential adding of absorbent to dialysate to further lower serum and dialysate potassium was examined. Serum potassium level of 8 mmol/L and dialysate with an initial potassium concentration of 4 mmol/l was used to decrease the initial serum-dialysate concentration gradient. As shown in FIG. 75, after 20 minutes of dialysis, the dialysate potassium levels increased due to the recirculation of dialysate. Thus, a dose of potassium adsorbent was added to the dialysate before allowing serum and dialysate to reach an equilibrium. Potassium was removed from the dialysate, and a similar potassium serum-dialysate concentration gradient was established after a dose of adsorbent. After 40 minutes of using slurry dialysate (60 minutes of dialysis), potassium concentration in the dialysate was on the rise; thus, another dose of potassium binder was added to the dialysate. The final serum potassium concentration was lowered to 2.3 mmol/l for demonstration purposes.

Safety: At the end of a hemodialysis experiment using a 20 g/l slurry solution, blood samples were oven-dried and concentrated. Energy-dispersive X-ray spectroscopy (EDS) was used for elemental analysis of dried-concentrated blood samples. Here, sodium zirconium cyclosilicate was used as a model potassium binder in the dialysate. Multiple samples and sample sites were scanned using EDS for zirconium (Zr) and silicon (Si) to check if the potassium binder had leached the blood. No zirconium (Zr) and silicon (Si) were detected in dried-concentrated blood samples. In another experiment, blood was replaced with deionized (DI) water and fluid was transferred from the dialysate container to the blood container. A slurry of 10 g/l solutions was used as a dialysate solution. Over a few batches, 2 liters of the slurry (10 g/l) were filtered using a Fresenius dialyzer (F160NR). No suspended particles were detected in the DI-Water using a turbidity meter. No trace amount of Zr or Si was detected. These results suggest an initially favorable safety profile of the nanoslurry material.

Adding nanoslurry to the dialysate allows for improved efficiency and efficacy of HD as measured by small molecule clearance without leaching into the blood has been shown. Commercially available binders can be added to improve the efficiency of dialysis has been shown. Dialysate can be modified to improve the efficiency of hemodialysis and potentially reduce single pass dialysate volume and even reuse dialysate.

Adsorbents, particularly those containing charcoal, have long been recognized to absorb uremic toxins. Much of this research has focused on charcoal hemoperfusion; however, this can cause hemolysis and binding to other blood components requiring modification of the charcoal. Few studies have tried to use adsorbents in the dialysate. Meyer et al. as shown that adding charcoal based adsorbent was able to bind protein bound uremic solutes such as indican and p-cresol. Their adsorbents however, did not bind solutes such as urea and creatinine. This study showed that this carbon-based adsorbent had avidity for solutes such as urea and creatinine. Furthermore, the removal of potassium and phosphorus can be further augmented by the addition of already available potassium and phosphate binders. This can allow for the personalization of solute removal depending on patients' dietary behaviors. Another unique advantage of adding adsorbent to the dialysate is that it does not require any modification to current existing hemodialysis membranes and will not require changes in the manufacturing process.

Although this work has been solely in vitro it has several potential advantages for patients. The first is that patients may require less medication binders if added to the dialysate. Patients also be able to get more dialytic clearance in the same amount of time improving symptoms. By tailoring potassium and avoiding larger potassium gradients potentially improving cardiac stability. This is the first attempt to simultaneously accommodate a lower serum-dialysate concentration gradient and manage the removal rate throughout the treatment. In addition, to improving patient outcomes, nanoslurry has potential to reduce water consumption by reducing dialysate consumption. Water consumption can be a significant barrier to home therapies and reducing the water consumption of hemodialysis can improve access to many patients. Further, this can be utilized in in-center hemodialysis potentially reducing the environmental impact of hemodialysis, especially in water-scare areas.

Nano-Slurry improved the efficiency in both in-vitro single pass and recirculated hemodialysis models. These findings need to be replicated in animal models; however, this shows that addition of nanoscale adsorbents improved the efficiency of dialysis and can be incorporated into current in-center hemodialysis and home dialysis machines. In the future, a novel hemodialysis machine that can recirculate dialysate, reducing water consumption for both in-center and home hemodialysis.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising porous particles, wherein the porous particles comprise an organic polymer and further comprise functional groups that selectively and reversibly bind blood components; wherein the porous particles have a core and a shell; and wherein the core and the shell have different porosity.

2. The composition of claim 1, wherein the porous particles have a Brunauer-Emmet-Teller surface area between 1.0 and 30 m²/g.

3. The composition of claim 1, wherein the organic polymer is selected from the group consisting of chitosan, cellulose, lignin, polyacrylic, polysulfone or it can be synthesized with monomers comprising a styrene, an acrylate, a divinylbenzene, and combinations, co-polymers, or block co-polymers thereof.

4. The composition of claim 1, wherein the porous particles have an average pore diameter between 5 and 35 nm.

5. The composition of claim 1, wherein the porous particles have an average diameter between 2 nm to 1000 μm.

6. The composition of claim 1, wherein the porous particles comprise at least one crosslinker.

7. The composition of claim 1, wherein the porous particles comprise a compound having a known affinity for a specific blood component.

8. The composition of claim 1, wherein the core of the porous particle comprises functional groups that selectively and reversibly bind specific electrolytes, proteins, metabolites, or enzymes; and the shell of the porous particles comprise at least one biocompatible polymer.

9. The composition of claim 1, wherein the composition further comprises saline and optionally comprises a dispersant.

10. A kit for adding a purification composition to a dialysis unit, the kit comprising: the composition of claim 1 dispersed or suspended in saline; a syringe having a volume sufficient to contain the composition of claim 1; and instructions for using the kit.

11. A packed column comprising the composition of claim 1.

12. A dialyzer comprising the composition of claim 1.

13. The dialyzer of claim 12, wherein the porous particles comprise functional groups that selectively and reversibly bind potassium (K⁺) or phosphate (PO₄³⁻).

14. A method of removing potassium or phosphate from the blood of a subject, the method comprising the steps of:
providing the dialyzer of claim 12; and
dialyzing the blood of the subject using the dialyzer.

15. A method of dispersing a medical composition in a filtration unit, the method comprising the steps of:
providing a vessel comprising a medical composition;
securing the vessel to a filtration unit; and
depositing the composition into the filtration unit;
wherein the medical composition comprises porous particles comprising at least one organic polymer;
the filtration unit comprises a filtration membrane having hollow fiber pores; and
the average diameter of the porous particles is larger than the hollow fiber pores of the filtration membrane.

16. The method of claim 15, wherein the vessel is a syringe.

17. The method of claim 15, wherein the filtration unit is a dialyzer for the dialysis of blood and blood products.

18. A system for the removal of a blood component from the blood of a patient, the system comprising:
a dialyzer unit having a blood inlet, a blood outlet, and dialysate inlet, and a dialysate outlet;
wherein the blood inlet and blood outlet are fluidly connected, and the dialysate inlet and the dialysate outlet are fluidly connected;
a dialysate containment vessel which is fluidly connected to the dialysate inlet port via a filtration line and a bypass line;
wherein the bypass line includes a bypass valve between the dialyzer unit and the dialysate vessel;
wherein the filtration line includes a filtration cartridge fluidly connected to the dialysate containment vessel and the dialysate inlet and a valve between the purification cartridge and the dialysate containment vessel; and
wherein the filtration cartridge comprises the composition of claim 1.

19. A method of dialyzing the blood of a subject in need thereof, the method comprising the steps of:
providing the system of claim 18;
providing a dialysate solution;
adding a blood component to the dialysate solution such that the blood component concentration of the dialysate solution is lower than the blood component level of the blood;
passing the dialysate solution through the bypass line of the system and through the dialyzer until the blood component level of the blood and dialysate have equilibrated; and
directing at least a portion of the dialysate solution through the purification cartridge.

20. A method of synthesizing polymer beads, the method comprising the steps of:
dispersing a polymer in a solvent to give a polymer solution;
adding a blowing agent to the polymer solution; and
treating the polymer solution with a base, acid, or microwaves, to form polymer beads.
